(19) United States
(12) Patent Application Publication
(10) Pub. No.: US 2022/0087961 A1
(43) Pub. Date: Mar. 24, 2022

(54) TREATMENT METHODS OF TRIPHENYL CALCILYTIC COMPOUNDS

(71) Applicant: CALCILYTIX THERAPEUTICS, INC., San Francisco, CA (US)

(72) Inventors: Simon BRUCE, San Francisco, CA (US); Jonathan FOX, San Francisco, CA (US); Ramei SANI-GROSSO, San Francisco, CA (US); Ali KOMEYLI, San Francisco, CA (US); Ananth SRIDHAR, San Francisco, CA (US); Mary Scott ROBERTS, San Francisco, CA (US)

(21) Appl. No.: 17/477,198

(22) Filed: Sep. 16, 2021

Related U.S. Application Data
(60) Provisional application No. 63/080,165, filed on Sep. 18, 2020.
(60) Provisional application No. 63/159,397, filed on Mar. 10, 2021.

(51) Int. Cl. A61K 31/195; A61P 3/14

(52) U.S. Cl. CPC A61K 31/195; A61P 3/14

(57) ABSTRACT

The present disclosure provides a method of treating an autosomal dominant hypocalcemia type 1 (ADH1) with a therapeutically effective amount of a compound of formula (I), in particular CLTX-305, wherein the therapeutically effective amount of the compound increases a blood calcium concentration (cCa) to a range of about 7.5 mg/dL to about 10.5 mg/dL, such as about 8.5 mg/dL to about 10.5 mg/dL. Also provided herein is a dosing finding method for treating an autosomal dominant hypocalcemia type 1 (ADH1) with a therapeutically effective amount of a compound of formula (I) or CLTX-305 according to one or more dosing regimens.

### CROSS-REFERENCES TO RELATED APPLICATIONS

This application claims priority to U.S. Provisional Application No. 63/080,165 filed Sep. 18, 2020 and U.S. Provisional Application No. 63/159,397 filed Mar. 10, 2021, each of which is incorporated in its entirety for all purposes.

### STATEMENT AS TO RIGHTS TO INVENTIONS MADE UNDER FEDERALLY SPONSORED RESEARCH AND DEVELOPMENT

Not Applicable

### REFERENCE TO A “SEQUENCE LISTING,” A TABLE, OR A COMPUTER PROGRAM LISTING APPENDIX SUBMITTED ON A COMPACT DISK

Not Applicable

### BACKGROUND

Autosomal dominant hypocalcemia Type 1 (ADH1) is a rare disorder of systemic calcium homeostasis caused by activating mutations of the gene encoding the calcium-sensing receptor (CASR) leading to hypocalcemia (Hannan et. al. Human molecular genetics, 2012, 21 (12), p. 2768-2778; and Hofer et. al., Nature reviews: Molecular cell biology, 2003, 4 (7), p. 530-538). The calcium-sensing receptor (CaSR) plays the dominant role in regulating systemic calcium homeostasis by controlling parathyroid hormone (PTH) secretion and urinary calcium excretion in response to variations in extracellular calcium concentrations (Hofer et. al., Nature reviews: Molecular cell biology, 2003; and Gunn et. al., Ann Clin Biochem, 2004, 41 (Pt 6):p. 441-58). Negative allosteric modulators of the CaSR, (so-called calcilytic agents) may represent a potential targeted therapy for ADH1.

The prevalence of ADH1 is uncertain and has previously been estimated to occur in approximately of 1 per 70,000 (Gunn et al., Ann. Clin. Biochem., 2004) or 3.9 per 100,000 (Dershem et. al., The American Journal of Human Genetics, 2020, 106, 1-14). By any available estimate it qualifies as a Rare Disease according to the applicable criteria of the United States Food and Drug Administration and other competent health authorities like the European Medicines Agency, and is recognized as rare by the United States National Institutes of Health (NIH) Office of Rare Disease Research (Genetic and Rare Disease (GARD) Number: 2877) and the European rare disease consortium Orphanet (Orpha Number: 428).

ADH1 is characterized by variable degrees of hypocalcemia with abnormally low levels of parathyroid hormone (PTH), hyperphosphatemia and low magnesium levels usually with persistent hypercalciuria (Roszko et. al., Frontiers in Physiology, 2016, 7, p. 458). Symptoms of hypocalcemia most commonly include paresthesia, muscle spasms, cramps, tetany, circumoral numbness, and can be of variable intensity including inducing seizures. Hypocalcemia can also present with laryngospasm, neuromuscular irritability, cognitive impairment, personality disturbances, a prolonged QT interval on the surface electrocardiogram that may be associated with an increased risk of ventricular tachyarrhythmias, electrocardiographic changes that mimic myocardial infarction, and/or overt heart failure.

In patients with ADH1, hypocalcemia occurs primarily due to increased sensitivity of the CaSR to extracellular ionized calcium which suppresses iPTH-secretion and leads to lower levels of 1,25-(OH)₂-vitamin D (also referred to as 1,25-dihydroxyvitamin D3; decreasing calcium absorption from the gut) and lower levels of calcium reabsorption in the kidney (leading to relative hypercalciuria). Hypercalciuria is increased based on two mechanisms: reduced PTH-mediated reabsorption of calcium from the primary renal filtrate, and further decreased calcium reabsorption in the distal renal tubules where the mutated CaSR is over-sensitized to extracellular calcium concentrations. Furthermore, standard treatment with oral calcium and calcitriol (e.g., 1,25-dihydroxyvitamin D3) supplementation tends to worsen hypercalciuria which is associated with long-term morbidity such as nephrolithiasis, nephrocalcinosis, and chronic kidney disease that can progress to renal failure (Khan et. al., European journal of endocrinology, 2018; and Li et. al., Clinical interventions in aging, 2018, 13, p. 2443-2452).

For this reason, the consensus approach to management of ADH1 is to balance oral supplementation of calcium and calcitriol with the known high risk for renal calcifications, kidney stones, and kidney failure (Roszko et. al., Frontiers in physiology, 2016). This means the healthcare provider must help the patient find a regimen that can maintain the lowest serum calcium concentrations compatible with symptom relief to minimize hypercalciuria. Thiazide diuretics are sometimes added for their modest urinary calcium lowering effects but can pose a risk of hypokalemia.

Experimental treatment with PTH(1-34) in ADH1 subjects was able to correct serum calcium but did not abrogate hypercalciuria (Winer et. al., The Journal of clinical endocrinology and metabolism, 2012, 97 (2), p. 391-399; Winer et. al., The Journal of pediatrics, 2014, 165 (3), 556-63; and Gafni et. al., Journal of bone and mineral research, 2015, 30 (11), p. 2112-2118). Exogenous PTH(1-84) is approved for the orphan indication of hypoparathyroidism but the clinical study in patients with established hypoparathyroidism that supported approval excluded patients with hypoparathyroidism due to calcium sensing receptor mutations (Chomsky et. al., World Journal of Surgery, 2018, 42 (2), p. 431-436; and Natpara Product Insert).

Triphenyl calcilytic compounds refer to a class of compounds having a calcium-sensing receptor antagonistic action, as disclosed in U.S. Pat. No. 7,304,174 and represented by the following formula:

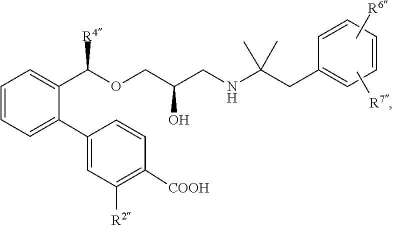

wherein R^{2″} is a C₁₋₆ alkyl group; R^{4″} is a methyl group or a cyclopropyl group; R^{6″} is a halogen atom or a C₁₋₆ alkyl group, and R^{7″} is a hydrogen atom, a halogen atom, a C₁₋₆ alkyl group, a C₁₋₆ alkoxy group, or a halo C₁₋₆ alkyl group, an optically active form thereof, a pharmaceutically acceptable salt thereof, or an optically active form of the salt thereof. In particular, the triphenyl calcilytic compound is represented by formula (I):

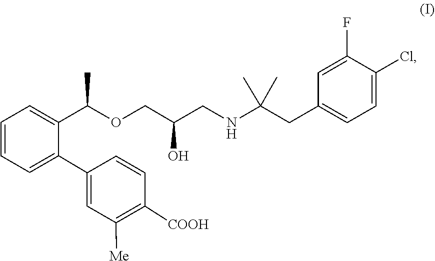 [SMILES: Cc1cc(-c2ccccc2[C@@H](C)OC[C@H](O)CNC(C)(C)Cc2ccc(Cl)c(F)c2)ccc1C(=O)O]

a solvate, a hydrate, a pharmaceutically acceptable salt, or a combination thereof. In certain embodiments, the compound of formula (I) is CLTX-305 represented by the formula:

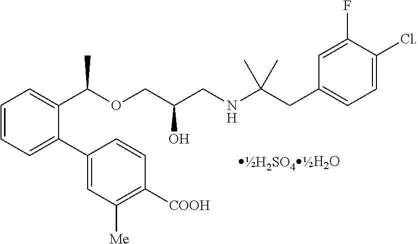 [SMILES: Cc1cc(-c2ccccc2[C@@H](C)OC[C@H](O)CNC(C)(C)Cc2ccc(Cl)c(F)c2)ccc1C(=O)O]

Previously, CLTX-305 (known previously as JTT-305 or MK-5442) was developed as a treatment for osteoporosis by Japan Tobacco Inc., (JTI) and Merck, Sharp & Dohme Corp., (Merck). Healthy volunteers and postmenopausal women with osteoporosis participated in a 1,766-subject program of which approximately 1,280 were exposed to JTT-305 in eight phase-1 and four phase-2 studies including exposures up to 52 weeks. Despite early data demonstrating a potential net benefit on bone formation, late phase trials failed to demonstrate efficacy on endpoints of bone mineral density (BMD) in postmenopausal women with osteoporosis.

Hypercalcemia was identified as an on-target but dose-limiting side effect in the osteoporosis program as reviewed below. Hypercalcemia posed a safety issue in the osteoporosis program, whereas increase in blood calcium is considered a marker for efficacy in the ADH1 program.

The compound of formula (I) or CLTX-305 may prove to be a therapy uniquely targeted to the underlying pathogenesis of altered calcium homeostasis in patients with ADH1 where resetting the CaSR “set-point” might normalize serum calcium with minimal requirements for oral calcium and calcitriol supplementation and without increasing the risk of iatrogenic chronic hypercalciuria.

As such, there exists a need for the development of CLTX-305 as a targeted therapy to treat hypocalcemia and disturbed calcium homeostasis in patients with chronic hypocalcemia due to activating mutations in the calcium sensing receptor (CaSR).

### SUMMARY

In one aspect, the present disclosure provides a method of treating an autosomal dominant hypocalcemia type 1 (ADH1). The method includes administering to a subject in need thereof a therapeutically effective amount of a compound represented by formula (I):

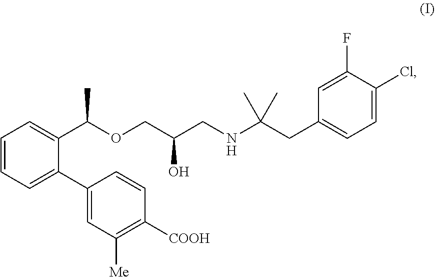 [SMILES: Cc1cc(-c2ccccc2[C@@H](C)OC[C@H](O)CNC(C)(C)Cc2ccc(Cl)c(F)c2)ccc1C(=O)O]

or a solvate, a hydrate, a pharmaceutically acceptable salt, or a combination thereof, wherein the therapeutically effective amount of the compound or the solvate, hydrate, pharmaceutically acceptable salt, or combination thereof, increases a blood calcium concentration (cCa) to a range of about 7.5 milligrams per deciliter (mg/dL) to about 10.5 mg/dL, such as from about 8.5 mg/dL to about 10.5 mg/dL.

In another aspect, the present disclosure provides a method of treating an autosomal dominant hypocalcemia type 1 (ADH1), where the method includes administering to a subject in need thereof a compound represented by formula (I):

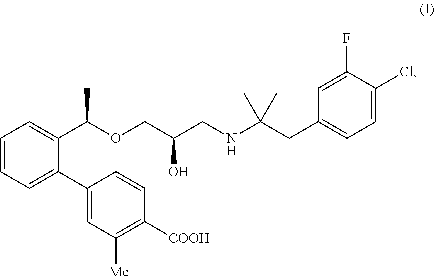 [SMILES: Cc1cc(-c2ccccc2[C@@H](C)OC[C@H](O)CNC(C)(C)Cc2ccc(Cl)c(F)c2)ccc1C(=O)O]

or a solvate, a hydrate, a pharmaceutically acceptable salt, or a combination thereof, according to one or more dosing regimens including a first dosing regimen, a second dosing regimen, and/or a third dosing regimen, wherein:
1) the first dosing regimen includes administering a first therapeutically effective amount of the compound or the solvate, hydrate, pharmaceutically acceptable salt, or combination thereof, wherein the first therapeutically effective amount increases a blood calcium concentration (cCa) up to a maximum cCa of about 10.5 milligrams per deciliter (mg/dL);
2) the second dosing regimen includes administering a second therapeutically effective amount of the compound or the solvate, hydrate, pharmaceutically acceptable salt, or combination thereof, wherein the second therapeutically effective amount titrates the blood calcium concentration (cCa) to a range of about 7.5 mg/dL to about 10.5 mg/dL; and
3) the third dosing regimen includes administering a third therapeutically effective amount of the compound or the solvate, hydrate, pharmaceutically acceptable salt, or combination thereof, wherein the third therapeutically effective amount maintains the blood calcium concentration (cCa) in a range of about 7.5 mg/dL to about 10.5 mg/dL for a period of at least 12 weeks.

### BRIEF DESCRIPTION OF THE DRAWINGS

FIG. 1 shows an overall study design of a Phase 2b, open-label dose-ranging study evaluating the safety, tolerability, and efficacy of CLTX-305 to maintain normalized albumin-corrected blood calcium (cCa) in subjects with hypocalcemia due to ADH1.

FIG. 2 shows an overview scheme of the Phase 2b study.

FIG. 3 shows a detailed scheme for Periods 1 and 2 (single and multiple ascending dose testing) of the study.

FIG. 4 shows a detailed scheme for Period 3 of the study.

FIG. 5 shows a dosing guidance algorithm for Period 1 of the study.

FIG. 6 shows a dosing guidance algorithm for Period 2 of the study.

FIGS. 7A-7D show mineral homeostasis normalized during Period 1 of the study.

FIG. 7A: Blood calcium (mg/dL); FIG. 7B: Intact PTH (pg/mL); FIG. 7C: Blood phosphorous (mg/dL); and FIG. 7D: 24 h Urine calcium (mg/24 h).

FIG. 8 shows pharmacokinetic profile of CLTX-305 in human during Period 1 of the study.

FIGS. 9A-9D show blood mineral levels following 5-day dosing of CLTX-305 for individual subject and as an average. FIG. 9A: Blood calcium (mg/dL); FIG. 9B: Intact PTH (pg/mL); FIG. 9C: Blood phosphorous (mg/dL); and FIG. 9D: 24 h Urine calcium (mg/day).

FIGS. 10A and 10B show a dosing summary for oral CLTX-305 in Periods 1 and 2 of the study.

### DETAILED DESCRIPTION

### I. General

The present disclosure provides a method of treating an autosomal dominant hypocalcemia type 1 (ADH1) with a therapeutically effective amount of a compound of formula (I), in particular CLTX-305, wherein the therapeutically effective amount of the compound increases a blood calcium concentration (cCa) to a range of about 7.5 mg/dL to about 10.5 mg/dL, such as from about 8.5 mg/dL to about 10.5 mg/dL. Also provided herein is a dosing finding method for treating an autosomal dominant hypocalcemia type 1 (ADH1) with a therapeutically effective amount of a compound of formula (I) according to one or more dosing regimens (e.g., first, second, and third dosing regimens). In particular, a therapeutically effective amount of the compound of formula (I) (e.g., CLTX-305) reduces symptoms associated with hypocalcemia and minimizes hypercalciuria in ADH1 subjects.

### II. Definitions

Unless specifically indicated otherwise, all technical and scientific terms used herein have the same meaning as commonly understood by those of ordinary skill in the art to which this disclosure belongs. In addition, any method or material similar or equivalent to a method or material described herein can be used in the practice of the present disclosures. For purposes of the present disclosure, the following terms are defined.

“Tablet” refers to solid pharmaceutical formulations with and without a coating. The term “tablet” also refers to tablets having one, two, three or even more layers, wherein each of the before mentioned types of tablets may be without or with one or more coatings. In some embodiments, tablets of the present disclosure can be prepared by roller compaction or other suitable means known in the art. The term “tablet” also comprises mini, melt, chewable, effervescent, and orally disintegrating tablets. Tablets include CLTX-305 and one or more pharmaceutical excipients selected from one or more fillers, one or more binders, one or more glidants, one or more disintegrants, one or more surfactants, one or more binders, and one or more lubricants. Optionally, a coating agent can be also included. For the purposes of calculating percent weight of the tablet formulation, the amount of coating agent is not included in the calculation. That is, the percent weights reported herein are of the uncoated tablet.

“Pharmaceutically acceptable excipient” refers to a substance that aids the administration of an active agent to and absorption by a subject. Pharmaceutical excipients useful in the present disclosure include, but are not limited to, binders, fillers, glidants, disintegrants, surfactants, lubricants, coatings, sweeteners, flavors, and colors. One of skill in the art will recognize that other pharmaceutical excipients are useful in the present disclosure.

“Administering” refers to therapeutic provision of the compound or a form thereof to a subject, such as by oral administration.

“Patient” or “subject” refers to a living organism suffering from or prone to a disease or condition that can be treated by administration of a pharmaceutical composition as provided herein. Non-limiting examples include humans, non-human primates (e.g., monkeys), goats, pigs, sheep, cows, deer, horses, bovines, rats, mice, rabbits, hamsters, guinea pigs, cats, dogs, and other non-mammalian animals. In some embodiments, the subject is human. In some embodiments, a subject is an adult (e.g., at least 18 years of age). In some embodiments, the subject is less than 18 years of age. In some embodiments, the subject is between about 6 months to about 2 years of age, about 2 years to about 5 years of age, about 2 years to about 12 years of age, about 2 years to about 16 years of age, about 2 years to about 18 years of age, about 5 years to about 12 years of age, about 6 years to about 12 years of age, about 6 years of age to about 18 years of age, about 12 years of age to about 18 years of age, or any range therein.

“Therapeutically effective amount” refers to an amount of a compound or of a pharmaceutical composition useful for treating or ameliorating an identified disease or condition, or for exhibiting a detectable therapeutic or inhibitory effect. The exact amounts will depend on the purpose of the treatment, and will be ascertainable by clinicians, pharmacists, and the like (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

“Treat”, “treating,” and “treatment” refer to any indicia of success in the treatment or amelioration of an injury, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, assay (e.g., analysis of a fluid of a subject, such as blood, plasma, or urine), imaging analysis, neuropsychiatric exams, and/or a psychiatric evaluation.

“About” means a range of values including the specified value, which a person of ordinary skill in the art would consider reasonably similar to the specified value. In some embodiments, the term “about” means within a standard deviation using measurements generally acceptable in the art. In some embodiments, about means a range extending to +/−10% of the specified value. In some embodiments, about means the specified value.

“Salt” refers to acid or base salts of the compounds of the present disclosure. Illustrative examples of pharmaceutically acceptable acid addition salts are mineral acid (hydrochloric acid, hydrobromic acid, phosphoric acid, and the like) salts and organic acid (acetic acid, propionic acid, glutamic acid, citric acid and the like) salts. Examples of pharmaceutically acceptable base addition salts include sodium, potassium, calcium, ammonium, organic amino, or magnesium salt, or a similar salt. It is understood that the pharmaceutically acceptable salts are non-toxic. Additional information on suitable pharmaceutically acceptable salts can be found in Remington's Pharmaceutical Sciences, 17th ed., Mack Publishing Company, Easton, Pa., 1985, which is incorporated herein by reference.

“Solvate” refers to a compound provided herein or a salt thereof, that further includes a stoichiometric or non-stoichiometric amount of solvent bound by non-covalent intermolecular forces. Where the solvent is water, the solvate is a hydrate.

“Hydrate” refers to a compound provided herein or a salt thereof, that is complexed with a water molecule. The compounds or salts thereof of the present disclosure can be complexed with ½ water molecule or from 1 to 10 water molecules.

Unless specifically indicated otherwise, the content of the compound of formula (I) in, e.g., a tablet formulation is calculated based on the normalized weight of the compound of formula (I) on a salt-free and anhydrous basis. That is, the salt and/or water content in the compound of formula (I) is not included in the calculation.

Unless specifically indicated otherwise, the content of CLTX-305 in, e.g., a tablet formulation is calculated based on normalized weight of the compound of formula (I) in a hemisulfate salt form. A theoretical content of the compound of formula (I) on a salt-free and anhydrous basis in a tablet formulation can be calculated as follows: “the content (or weight) of formula (I) on a salt-free and anhydrous basis=the content (or weight) of CLTX-305×0.90”. An actual content of the compound of formula (I) on a salt-free and anhydrous basis in a tablet formulation may be calculated according to a Certificate of Analysis (CoA) of CLTX-305 (e.g., purity, water content, etc.), therefore may vary slightly among batches of CLTX-305 from manufacturing.

### III. Methods

In one aspect, the present disclosure provides a method of treating an autosomal dominant hypocalcemia type 1 (ADH1). The method includes administering to a subject in need thereof a therapeutically effective amount of a compound represented by formula (I):

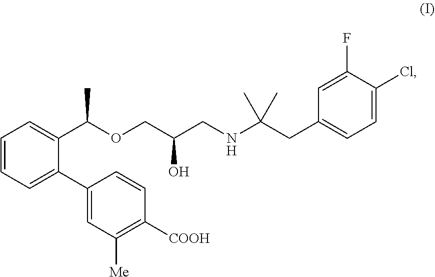 [SMILES: Cc1cc(-c2ccccc2[C@@H](C)OC[C@H](O)CNC(C)(C)Cc2ccc(Cl)c(F)c2)ccc1C(=O)O]

or a solvate, a hydrate, a pharmaceutically acceptable salt, or a combination thereof, wherein the therapeutically effective amount of the compound or the solvate, hydrate, pharmaceutically acceptable salt, or combination thereof, increases a blood calcium concentration (cCa) to a range of from about 7.5 mg/dL to about 10.5 mg/dL, such as from about 8.5 mg/dL to about 10.5 mg/dL.

For clinical lab assessment, a typical reference range of a blood calcium concentration (cCa) in human is from about 8.5 mg/dL to about 10.5 mg/dL.

### III-1: Compound of Formula (I)

The compound of formula (I) can be in a pharmaceutically acceptable salt form, in a zwitterionic form, or in a neutral form, each of which is optionally in a solvate or a hydrate form.

In some embodiments, a pharmaceutically acceptable acid addition salt of the compound of formula (I) is represented by formula (Ia):

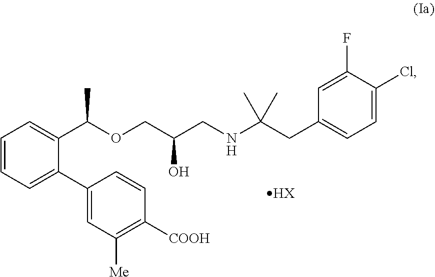

wherein HX is a pharmaceutically acceptable acid addition.

Examples of acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. In some embodiments, the compound of formula (I) is in a sulfate salt form. In some embodiments, the compound of formula (I) is in a hemisulfate salt form.

In some embodiments, a pharmaceutically acceptable base addition salt of the compound of formula (I) is represented by formula (Ib):

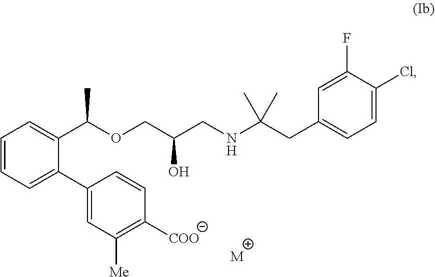

wherein M is a pharmaceutically acceptable cation of a base.

The base addition salts can be obtained by contacting the neutral form of the compound of formula (I) with a sufficient amount of the desired base, either neat or in a suitable inert solvent. Examples of pharmaceutically acceptable base addition salts include sodium, potassium, calcium, ammonium, organic amino, or magnesium salt, or a similar salt. In some embodiments, the compound of formula (I) is a sodium salt thereof.

In some embodiments, the compound of formula (I) is in a zwitterionic form having formula (Ic):

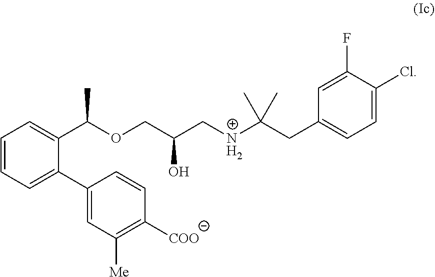

In some embodiments, the compound of formula (I) is in a neutral form.

In some embodiments, the compound of any one of formulae (I), (Ia), (Ib), and (Ic) is in a solvate and/or a hydrate form. In some embodiments, the compound of any one of formulae (I), (Ia), (Ib), and (Ic) is in a hydrate form.

In some embodiments, the compound of formula (I) is in a hemihydrate hemisulfate salt form as CLTX-305 represented by the formula:

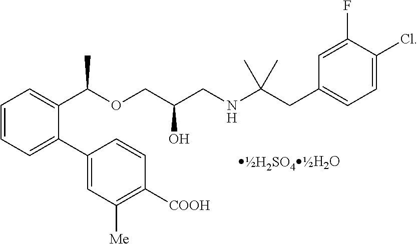 [SMILES: Cc1cc(-c2ccccc2[C@@H](C)OC[C@H](O)CNC(C)(C)Cc2ccc(Cl)c(F)c2)ccc1C(=O)O]

### III-2: Subject

The subject can have one or more symptoms of hypocalcemia, hypoparathyroidism, hypercalciuria, hyperphosphatemia, and/or hypomagnesemia. In some embodiments, the subject has hypocalcemia, hypoparathyroidism, hypercalciuria, hyperphosphatemia, hypomagnesemia, or a combination thereof. In some embodiments, the subject has hypocalcemia, hypoparathyroidism, hypercalciuria, hyperphosphatemia, or a combination thereof. In some embodiments, the subject has hypocalcemia. In some embodiments, the subject has hypoparathyroidism. In some embodiments, the subject has hyperphosphatemia. In some embodiments, the subject has hypercalciuria. In some embodiments, the subject has hypocalcemia and hypoparathyroidism. In some embodiments, the subject has hypocalcemia and hypercalciuria. In some embodiments, the subject has hypocalcemia, hypoparathyroidism, and hypercalciuria. In some embodiments, the subject has hypocalcemia, hypoparathyroidism, hypercalciuria, and hyperphosphatemia. In some embodiments, the subject has hypocalcemia, hypoparathyroidism, hypercalciuria, hyperphosphatemia, and hypomagnesemia.

In some embodiments, the subject has an activating mutation of the calcium-sensing receptor (CASR) gene.

In some embodiments, prior to the treatment with a compound of formula (I) (e.g., CLTX-305), or a form thereof, the subject does not have a vitamin D deficiency. In some embodiments, the subject has 25-hydroxy-vitamin D in blood at a level of at least about 25 nanograms per milliliter (ng/mL). In some embodiments, the subject has 25-hydroxy-vitamin D in blood at a level of from about 25 ng/mL to about 60 ng/mL. In some embodiments, the subject has 25-hydroxy-vitamin D in blood at a level of from about 30 ng/mL to about 60 ng/mL. In some embodiments, the subject has 25-hydroxy-vitamin D in blood at a level of from about 40 ng/mL to about 60 ng/mL.

In some embodiments, prior to administration of a compound of formula (I) (e.g., CLTX-305) or a form thereof, the subject has taken or is taking calcitriol and/or an oral calcium supplement regimen. In some embodiments, prior to administration of a compound of formula (I) (e.g., CLTX-305) or a form thereof, the subject is instructed to stop taking calcitriol and/or an oral calcium supplement regimen. In some embodiments, prior to administration of a compound of formula (I) (e.g., CLTX-305) or a form thereof, the subject is instructed to stop taking calcitriol. In some embodiments, prior to administration of a compound of formula (I) (e.g., CLTX-305) or a form thereof, the subject is instructed to stop taking an oral calcium supplement regimen. In some embodiments, the subject is instructed to stop taking calcitriol on Day −1, the day of admission for being treated with a compound of formula (I) (e.g., CLTX-305) or a form thereof, but to continue taking an oral calcium supplement regimen. In some embodiments, the subject is not treated with calcitriol while being treated with a compound of formula (I) (e.g., CLTX-305) or a form thereof. In some embodiments, the subject receives a daily calcium intake of at least about 1000 milligrams (mg) from diet and/or supplementation while being treated with a compound of formula (I) (e.g., CLTX-305) or a form thereof. In some embodiments, the subject receives a daily calcium intake of at least about 1000 mg from diet and supplementation while being treated with a compound of formula (I) (e.g., CLTX-305) or a form thereof.

In some embodiments, prior to administration of a compound of formula (I) (e.g., CLTX-305) or a form thereof, the subject has taken or is taking a thiazide diuretic. In some embodiments, the subject that has taken or is taking a thiazide diuretic may be treated with a compound of formula (I) (e.g., CLTX-305) or a form thereof if the subject discontinues the thiazide diuretic five (5) half-lives prior to the initiation of treatment with the compound of formula (I) (e.g., CLTX-305) or the form thereof and during the treatment period of the compound of formula (I) (e.g., CLTX-305) or the form thereof. In some embodiments, if the thiazide is being used as an antihypertensive, an alternative therapy for treating hypertension can be offered. Examples of oral thiazide diuretics include, but are not limited to, Chlorothiazide (Diuril), Chlorthalidone, Hydrochlorothiazide (Microzide), Indapamide, and Metolazone. In some embodiments, the subject is not treated with a thiazide diuretic for a period of five (5) half-lives of the thiazide diuretic prior to the treatment with a compound of formula (I) (e.g., CLTX-305) or a form thereof. In some embodiments, the subject is not treated with a thiazide diuretic for a period of five (5) half-lives of the thiazide diuretic prior to the treatment with a compound of formula (I) (e.g., CLTX-305) or a form thereof, wherein the thiazide diuretic is chlorothiazide (Diuril), chlorthalidone, hydrochlorothiazide (Microzide), indapamide, or metolazone. In some embodiments, the subject is not treated with a thiazide diuretic while being treated with a compound of formula (I) (e.g., CLTX-305) or a form thereof. In some embodiments, the subject is not treated with a thiazide diuretic while being treated with a compound of formula (I) (e.g., CLTX-305) or a form thereof, wherein the thiazide diuretic is chlorothiazide (Diuril), chlorthalidone, hydrochlorothiazide (Microzide), indapamide, or metolazone.

In some embodiments, prior to administration of a compound of formula (I) (e.g., CLTX-305) or a form thereof, the subject has taken or is taking a CYP3A4 inhibitor (e.g., a strong CYP3A4 inhibitor, such as clarithromycin, telithromycin, nefazodone, itraconazole, ketoconazole, atazanavir, darunavir, indinavir, lopinavir, nelfinavir, ritonavir, saquinavir, and tipranavir). In some embodiments, the subject that has taken or is taking a CYP3A4 inhibitor should, if clinically appropriate, discontinue such treatment at least 5 half-lives prior to initiation of treatment with a compound of formula (I) (e.g., CLTX-305) or a form thereof and during the treatment period of the compound of formula (I) (e.g., CLTX-305) or a form thereof. In some embodiments, the subject being treated with a CYP3A4 inhibitor (e.g., a strong CYP3A4 inhibitor) should remain on stable doses throughout the treatment of the compound of formula (I) (e.g., CLTX-305) or form thereof. In some embodiments, the subject is not treated with CYP3A4 inhibitor (e.g., a strong CYP3A4 inhibitor, such as clarithromycin, telithromycin, nefazodone, itraconazole, ketoconazole, atazanavir, darunavir, indinavir, lopinavir, nelfinavir, ritonavir, saquinavir, and tipranavir) for a period of five (5) half-lives of the CYP3A4 inhibitor prior to the treatment of a compound of formula (I) (e.g., CLTX-305) or a form thereof. In some embodiments, the subject is not treated with a strong CYP3A4 inhibitor for a period of five (5) half-lives of the strong CYP3A4 inhibitor prior to the treatment of a compound of formula (I) (e.g., CLTX-305) or a form thereof, wherein the strong CYP3A4 inhibitor is clarithromycin, telithromycin, nefazodone, itraconazole, ketoconazole, atazanavir, darunavir, indinavir, lopinavir, nelfinavir, ritonavir, saquinavir, or tipranavir. In some embodiments, the subject is not treated with a CYP3A4 inhibitor (e.g., a strong CYP3A4 inhibitor, such as clarithromycin, telithromycin, nefazodone, itraconazole, ketoconazole, atazanavir, darunavir, indinavir, lopinavir, nelfinavir, ritonavir, saquinavir, and tipranavir) while being treated with a compound of formula (I) (e.g., CLTX-305) or a form thereof. In some embodiments, the subject is not treated with a strong CYP3A4 inhibitor while being treated with CLTX-305, wherein the strong CYP3A4 inhibitor is clarithromycin, telithromycin, nefazodone, itraconazole, ketoconazole, atazanavir, darunavir, indinavir, lopinavir, nelfinavir, ritonavir, saquinavir, or tipranavir. In some embodiments, the subject is treated with a CYP3A4 inhibitor (e.g., a strong CYP3A4 inhibitor, such as clarithromycin, telithromycin, nefazodone, itraconazole, ketoconazole, atazanavir, darunavir, indinavir, lopinavir, nelfinavir, ritonavir, saquinavir, and tipranavir) at a stable dose while being treated with a compound of formula (I) (e.g., CLTX-305) or a form thereof. In some embodiments, the subject is treated with a strong CYP3A4 inhibitor at a stable dose while being treated with a compound of formula (I) (e.g., CLTX-305) or a form thereof, wherein the strong CYP3A4 inhibitor is clarithromycin, telithromycin, nefazodone, itraconazole, ketoconazole, atazanavir, darunavir, indinavir, lopinavir, nelfinavir, ritonavir, saquinavir, or tipranavir.

Further inclusion and exclusion criteria for subjects who may benefit from treatment with a compound of formula (I) (e.g., CLTX-305) or a form thereof, such as subjects enrolled in a Phase 2b, open-label dose-ranging study, are described in Example 5.

In some embodiments, the subject meets all of inclusion criteria of 1) to 7) as described in Example 5. In some embodiments, the subject meets all of inclusion criteria of 1) to 7) as described in Example 5, provided that the subject does not meet any one of exclusion criteria of 1) to 14) as described in Example 5.

### III-3: Therapeutically Effective Amount/Administration

In some embodiments, the therapeutically effective amount can be a total daily dosage of no more than about 1800 mg of the compound of formula (I) on a salt-free and anhydrous basis. In some embodiments, the therapeutically effective amount is a total daily dosage of from about 9 mg to about 1620 mg, from about 9 mg to about 1080 mg, from about 9 mg to about 810 mg, from 9 mg to about 540 mg, or from about 9 mg to about 324 mg of the compound of formula (I), on a salt-free and anhydrous basis, or any useful range therein.

In some embodiments, the therapeutically effective amount can be a total daily dosage of no more than about 2000 mg of CLTX-305. In some embodiments, the therapeutically effective amount is a total daily dosage of from about 10 mg to about 1800 mg, from about 10 mg to about 1200 mg, from about 10 mg to about 900 mg, from about 10 mg to about 600 mg, from about 10 mg to about 360 mg of CLTX-305, or from about 30 mg to about 180 mg of CLTX-305. In some embodiments, the therapeutically effective amount is a total daily dosage of about 10 mg, 20 mg, 30 mg, 40 mg, 60 mg, 90 mg, 120 mg, 180 mg, 360 mg, 480 mg, or 720 mg of CLTX-305. In some embodiments, the therapeutically effective amount is a total daily dosage of about 10 mg, 30 mg, 60 mg, 90 mg, 120 mg, 180 mg, 360 mg, 480 mg, or 720 mg of CLTX-305. In some embodiments, the therapeutically effective amount is a total daily dosage of about 10 mg, 60 mg, 90 mg, 120 mg, 180 mg, 360 mg, 480 mg, or 720 mg of CLTX-305. In some embodiments, the therapeutically effective amount is a total daily dosage of about 10 mg, 60 mg, 90 mg, 120 mg, 180 mg, 360 mg, or 480 mg of CLTX-305. In some embodiments, the therapeutically effective amount is a total daily dosage of about 10 mg, 60 mg, 90 mg, 120 mg, 180 mg, or 360 mg of CLTX-305. In some embodiments, the therapeutically effective amount is a total daily dosage of about 10 mg, 60 mg, 90 mg, 120 mg, or 180 mg of CLTX-305. In some embodiments, the therapeutically effective amount is a total daily dosage of about 10 mg, 30 mg, 90 mg, 120 mg, or 180 mg.

In general, the compound of formula (I) or CLTX-305 can be administered orally. In some embodiments, the compound of formula (I) or CLTX-305 is administered orally. In some embodiments, the compound of formula (I) is administered orally. In some embodiments, CLTX-305 is administered orally. In some embodiments, the compound of formula (I) in a tablet formulation is administered orally. In some embodiments, CLTX-305 in a tablet formulation is administered orally.

In general, the compound of formula (I) or CLTX-305 can be administered once or multiple times (e.g., 2, 3, 4, or more times) daily. In some embodiments, the compound of formula (I) or CLTX-305 is administered once, twice, three times, or four times daily. In some embodiments, the compound of formula (I) or CLTX-305 is administered once daily. In some embodiments, the compound of formula (I) or CLTX-305 is administered twice daily. In some embodiments, CLTX-305 is administered once, twice, three times, or four times daily. In some embodiments, CLTX-305 is administered once daily. In some embodiments, CLTX-305 is administered twice daily. In some embodiments, CLTX-305 is administered three times daily. In some embodiments, CLTX-305 is administered four times daily.

The compound of formula (I) can be in an oral dosage form in one or more dosage strengths, where the compound of formula (I) is present in an amount of at least about 1 mg, 5 mg, 10 mg, 20 mg, 30 mg, 50 mg, 90 mg, 100 mg, 120 mg, 180 mg, 200 mg, 300 mg, 400 mg, or 500 mg, on a salt-free and anhydrous basis. In some embodiments, the oral dosage form is a tablet formulation in one or more dosage strengths. In some embodiments of the tablet formulation, the compound of formula (I) is present in an amount of from 1 to 1000 mg, from 1 to 750 mg, from 1 to 500 mg, from 1 to 250 mg, from 30 to 1000 mg, from 30 to 750 mg, from 30 to 500 mg, from 30 to 200 mg, from 30 to 180 mg, from 30 to 120 mg, from 30 to 90 mg, from 50 to 1000 mg, from 50 to 750 mg, from 50 to 500 mg, from 50 to 250 mg, from 100 to 1000 mg, from 100 to 750 mg, from 100 to 500 mg, from 100 to 250 mg, from 200 to 1000 mg, from 200 to 750 mg, from 200 to 500 mg, from 300 to 1000 mg, from 300 to 750 mg, from 300 to 500 mg, from 400 to 1000 mg, from 400 to 750 mg, from 500 to 1000 mg, from 500 to 750 mg, from 600 to 1000 mg, from 5 to 250 mg, or from 5 to 100 mg in each tablet, on a salt-free and anhydrous basis. In some embodiments of the tablet formulation, the compound of formula (I) is present in an amount of about 5 mg, 10 mg, 30 mg, 60 mg, 90 mg, 100 mg, 120 mg, 150 mg, 180 mg, 200 mg, 250 mg, 300 mg, 400 mg, 500 mg, 600 mg, 700 mg, 800 mg, 900 mg, or 1000 mg in each tablet, on a salt-free and anhydrous basis. In some embodiments of the tablet formulation, the compound is present in an amount of about 10 mg, 30 mg, 60 mg, 90 mg, 100 mg, 120 mg, 150 mg, 180 mg, 200 mg, 300 mg, 400 mg, 500 mg, or 700 mg in each tablet, on a salt-free and anhydrous basis.

CLTX-305 can be in an oral dosage form in one or more dosage strengths, where CLTX-305 is present in an amount of at least about 1 mg, 5 mg, 10 mg, 20 mg, 30 mg, 50 mg, 90 mg, 100 mg, 120 mg, 180 mg, 200 mg, 300 mg, 400 mg, or 500 mg. In some embodiments, the oral dosage form is a tablet formulation in one or more dosage strengths. In some embodiments of the tablet formulation, CLTX-305 is present in an amount of from 1 to 1000 mg, from 1 to 750 mg, from 1 to 500 mg, from 1 to 250 mg, from 30 to 1000 mg, from 30 to 750 mg, from 30 to 500 mg, from 30 to 200 mg, from 30 to 180 mg, from 30 to 120 mg, from 30 to 90 mg, from 50 to 1000 mg, from 50 to 750 mg, from 50 to 500 mg, from 50 to 250 mg, from 100 to 1000 mg, from 100 to 750 mg, from 100 to 500 mg, from 100 to 250 mg, from 200 to 1000 mg, from 200 to 750 mg, from 200 to 500 mg, from 300 to 1000 mg, from 300 to 750 mg, from 300 to 500 mg, from 400 to 1000 mg, from 400 to 750 mg, from 500 to 1000 mg, from 500 to 750 mg, from 600 to 1000 mg, from 5 to 250 mg, or from 5 to 100 mg in each tablet. In some embodiments of the tablet formulation, CLTX-305 is present in an amount of about 5 mg, 10 mg, 30 mg, 60 mg, 90 mg, 120 mg, 180 mg, 240 mg, 300 mg, 360 mg, 420 mg, 480 mg, 540 mg, 600 mg, 660 mg, or 720 mg in each tablet. In some embodiments of the tablet formulation, CLTX-305 is present in an amount of about 10 mg, 30 mg, 60 mg, 90 mg, 120 mg, 180 mg, 240 mg, 360 mg, or 720 mg in each tablet.

In some embodiments, CLTX-305 is administered twice daily to provide a total daily dosage of no more than about 2000 mg of CLTX-305. In some embodiments, CLTX-305 is administered twice daily to provide a total daily dosage of from about 10 mg to about 1800 mg, from about 10 mg to about 1200 mg, from about 10 mg to about 900 mg, from about 10 mg to about 600 mg, from about 10 mg to about 360 mg of CLTX-305, or from about 30 mg to about 180 mg of CLTX-305. In some embodiments, CLTX-305 is administered twice daily to provide a total daily dosage of from about 10 mg to about 360 mg of CLTX-305. In some embodiments, CLTX-305 is administered twice daily to provide a total daily dosage of about 10 mg, 20 mg, 30 mg, 40 mg, 60 mg, 90 mg, 120 mg, 180 mg, 360 mg, 480 mg, or 720 mg of CLTX-305. In some embodiments, CLTX-305 is administered twice daily to provide a total daily dosage of about 10 mg, 30 mg, 60 mg, 90 mg, 120 mg, 180 mg, 360 mg, 480 mg, or 720 mg of CLTX-305.

### III-4: Efficacy

A Phase 2b, open-label dose-ranging study can evaluate the safety, tolerability, and efficacy of CLTX-305 to maintain normalized albumin-corrected blood calcium (cCa) in subjects with hypocalcemia due to ADH1, as summarized in Example 1 and detailed in Examples 2-8.

Administration of a therapeutically effective amount of the compound of formula (I) or CLTX-305 can mitigate symptoms associated with hypocalcemia. In some embodiments, administration of a therapeutically effective amount of the compound of formula (I) or CLTX-305 increases a blood calcium concentration (cCa) to a range of about 7.5 mg/dL to about 10.5 mg/dL, such as about 8.5 mg/dL to about 10.5 mg/dL. In some embodiments, administration of a therapeutically effective amount of the compound of formula (I) or CLTX-305 maintains a blood calcium concentration (cCa) in a range of about 7.5 mg/dL to about 10.5 mg/dL, such as from 8.5 mg/dL to about 10.5 mg/dL, for a period of at least 12 weeks. In some embodiments, administration of a therapeutically effective amount of the compound of formula (I) or CLTX-305 increases the blood calcium concentration (cCa) by at least about 1 mg/dL over a dosing interval. In some embodiments, administration of a therapeutically effective amount of CLTX-305 increases a blood calcium concentration (cCa) to a range of about 7.5 mg/dL to about 10.5 mg/dL, such as about 8.5 mg/dL to about 10.5 mg/dL. In some embodiments, administration of a therapeutically effective amount of CLTX-305 maintains a blood calcium concentration (cCa) in a range of about 7.5 mg/dL to about 10.5 mg/dL, such as about 8.5 mg/dL to about 10.5 mg/dL, for a period of at least 12 weeks. In some embodiments, administration of a therapeutically effective amount of CLTX-305 increases the blood calcium concentration (cCa) by at least about 1 mg/dL over a dosing interval.

In some embodiments, administration of a therapeutically effective amount of the compound of formula (I) or CLTX-305 increases intact parathyroid hormone (iPTH) in blood to a clinical reference range, typically between about 15-65 picograms per milliliter (pg/mL). In some embodiments, administration of a therapeutically effective amount of the compound of formula (I) or CLTX-305 increases intact parathyroid hormone (iPTH) in blood to a peak level of about 150 to about 300 pg/mL. In some embodiments, administration of a therapeutically effective amount of CLTX-305 increases intact parathyroid hormone (iPTH) in blood to a peak level of about 150 to about 300 pg/mL. In some embodiments, administration of a therapeutically effective amount of the compound of formula (I) or CLTX-305 increases intact parathyroid hormone (iPTH) in blood to an elevated level of at least about 50 pg/mL. In some embodiments, administration of a therapeutically effective amount of CLTX-305 increases intact parathyroid hormone (iPTH) in blood to an elevated level of at least about 50 pg/mL. In some embodiments, the iPTH is maintained at the elevated level of at least about 50 pg/mL for a period of 1-12 hours. In some embodiments, the iPTH is maintained at the elevated level of at least about 50 pg/mL for a period of 6-12 hours. In some embodiments, the iPTH is maintained at the elevated level of at least about 50 pg/mL for a period of about 12 hours.

Administration of a therapeutically effective amount of the compound of formula (I) can minimize the extent of hypercalciuria. In some embodiments, administration of a therapeutically effective amount of the compound of formula (I) or CLTX-305 decreases an elevated urinary calcium level. In some embodiments, administration of a therapeutically effective amount of the compound of formula (I) or CLTX-305 decreases an elevated urinary calcium level over a dosing interval or over a period of 24 hours. In some embodiments, administration of a therapeutically effective amount of CLTX-305 decreases an elevated urinary calcium level over a dosing interval or over a period of 24 hours. In some embodiments, administration of a therapeutically effective amount of CLTX-305 decreases an elevated urinary calcium level over a period of 24 hours. In some embodiments, administration of a therapeutically effective amount of the compound of formula (I) or CLTX-305 increases a urinary calcium clearance over a dosing interval or over a period of 24 hours. In some embodiments, administration of a therapeutically effective amount of CLTX-305 increases a urinary calcium clearance. In some embodiments, administration of a therapeutically effective amount of CLTX-305 increases a urinary calcium clearance over a dosing interval or over a period of 24 hours. In some embodiments, administration of a therapeutically effective amount of CLTX-305 increases a urinary calcium clearance over a period of 24 hours. In some embodiments, administration of a therapeutically effective amount of the compound of formula (I) or CLTX-305 decreases an elevated calcium/creatinine clearance ratio to a normal range. In some embodiments, administration of a therapeutically effective amount of the compound of formula (I) or CLTX-305 decreases an elevated calcium/creatinine clearance ratio to a normal range of less than about 0.02. In some embodiments, administration of a therapeutically effective amount of CLTX-305 decreases an elevated calcium/creatinine clearance ratio to a normal range of less than about 0.02.

Administration of a therapeutically effective amount of the compound of formula (I) or CLTX-305 can minimize the extent of hyperphosphatemia. In some embodiments, administration of a therapeutically effective amount of the compound of formula (I) or CLTX-305 decreases a blood phosphate level to a normal range. In some embodiments, administration of a therapeutically effective amount of the compound of formula (I) or CLTX-305 decreases a blood phosphate level to a normal range of from about 2.5 to about 4.5 mg/dL. In some embodiments, administration of a therapeutically effective amount of CLTX-305 decreases a blood phosphate level to a normal range of from about 2.5 to about 4.5 mg/dL.

Administration of a therapeutically effective amount of the compound of formula (I) or CLTX-305 can minimize the extent of hypomagnesium. In some embodiments, administration of a therapeutically effective amount of the compound of formula (I) or CLTX-305 increases a blood magnesium level to a normal range. In some embodiments, administration of a therapeutically effective amount of the compound of formula (I) or CLTX-305 increases a blood magnesium level to a normal range of from about 1.7 to about 2.2 mg/dL. In some embodiments, administration of a therapeutically effective amount of CLTX-305 increases a blood magnesium level to a normal range of from about 1.7 to about 2.2 mg/dL.

Blood and urine samplings can be performed to evaluate pharmacokinetic (PK) and pharmacodynamics (PD) in the subject (e.g., the patient) being treated with CLTX-305. In some embodiments, the subject is evaluated for 1) Pharmacodynamic endpoints measured over time up to 24 weeks (final visit); 2) Blood calcium—Absolute levels and change from baseline in cCa; 3) Urinary calcium clearance (fractional excretion and 24-hour total excretion); 4) Serum levels of 1,25-(OH)₂ Vitamin D; 5) Blood samples for magnesium, phosphate, creatinine; 6) Urine samples for pH, magnesium, phosphate, sodium, potassium, creatinine, cAMP, citrate; 7) Bone resorption markers collagen cross-linked C-telopeptide (CTx); 8) Bone formation markers—blood procollagen type 1 N-propeptide (P1NP); 9) PK parameters: maximum plasma concentration (Cmax), time to maximum plasma concentration (tmax), apparent terminal half-life (t½), area under the concentration-time curve (AUC) from time 0 to the last measurable time point (AUC(0-t)), AUC from time 0 to 24 hours (AUC(0-24)), AUC extrapolated to infinity (AUC(0-inf)) following single-doses; and/or 10) steady state PK parameters: Cmax, trough concentration (Ctrough), and AUC over the dosing interval AUC(0-tau).

In some embodiments, the subject is further evaluated for a level of serum bone markers, 1, 25-hydroxy-vitamin D, cAMP, albumin, potassium (K), creatine kinase (CK), and/or creatinine in blood. Serum bone markers can be blood collagen cross-linked C-telopeptide (CTx) and/or blood procollagen type 1 N-propeptide (P1NP). In some embodiments, serum bone markers are blood collagen cross-linked C-telopeptide (CTx) and/or blood procollagen type 1 N-propeptide (P1NP). In some embodiments, serum bone markers are blood collagen cross-linked C-telopeptide (CTx) and blood procollagen type 1 N-propeptide (P1NP).

In some embodiments, the blood is taken over a dosing interval or over a period of about 24 hours. In some embodiments, the blood is taken over a dosing interval or over a period of about 17 hours. In some embodiments, the blood is taken over a dosing interval or over a period of about 17 hours, wherein pharmacokinetic (PK) profiles, iPTH, serum bone markers (i.e., CTX, P1NP), 1, 25-hydroxy-vitamin D, cAMP, calcium (Ca), magnesium (Mg), phosphate (P), potassium (K), albumin, creatinine (Cr), or a combination are evaluated. In some embodiments, the blood is taken over a dosing interval or over a period of about 13 hours, wherein pharmacokinetic (PK) profiles are evaluated. In some embodiments, the blood is taken over a dosing interval or over a period of about 17 hours, wherein iPTH is evaluated. In some embodiments, the blood is taken over a dosing interval or over a period of about 13 hours, wherein serum bone markers (i.e., CTX, and P1NP) are evaluated. In some embodiments, the blood is taken over a dosing interval or over a period of about 17 hours, wherein 1, 25-hydroxy-vitamin D and cAMP are evaluated. In some embodiments, the blood is taken over a dosing interval or over a period of about 17 hours, wherein calcium (Ca), magnesium (Mg), phosphate (P), creatinine (Cr), and albumin are evaluated. In some embodiments, the blood is taken over a dosing interval or over a period of about 13 hours, wherein potassium (K) and creatinine (Cr) are evaluated.

In some embodiments, the subject is further evaluated for a level of magnesium (Mg), phosphate (P), sodium (Na), potassium (K), creatinine (Cr), cAMP, and/or citrate in urine, and/or a pH value of the urine.

In some embodiments, the urine is taken over a dosing interval or over a period of 24 hours. In some embodiments, the urine is taken over a dosing interval or over a period of 24 hours, wherein a level of calcium (Ca), magnesium (Mg), phosphate (P), creatinine (Cr), and/or cAMP in the urine; a pH value of the urine; or a combination thereof are evaluated. In some embodiments, the urine is taken at 0 hours and 24 hours, wherein a level of calcium (Ca), magnesium (Mg), phosphate (P), sodium (Na), potassium (K), creatinine (Cr), and/or citrate in the urine; a pH value of the urine; or a combination thereof are evaluated.

In some embodiments, the subject is evaluated by one or more tests including blood analyses, urine analyses, and/or hematology tests. Examples of such tests are described in Table 1, Table 2, and Table 3 of Example 1. In some embodiments, blood and urine sampling are scheduled according to Table 4A-Table 4C, Table 5, Table 6A-Table 6C, and Table 7 of Example 1.

In some embodiments, administration of a therapeutically effective amount of the compound of formula (I) or CLTX-305 maintains the blood calcium concentration (cCa) in a range of about 8.5 to about 10.5 mg/dL for a period of at least 12 weeks without substantially adjusting the total daily dosage. In some embodiments, administration of a therapeutically effective amount of the compound of formula (I) or CLTX-305 maintains the blood calcium concentration (cCa) in a range of about 8.5 to about 10.5 mg/dL for a period of at least 24 weeks without substantially adjusting the total daily dosage.

### III-5: Methods of Dosing Regiments

In another aspect, the present disclosure provides a method of treating an autosomal dominant hypocalcemia type 1 (ADH1), where the method includes administering to a subject in need thereof a compound represented by formula (I):

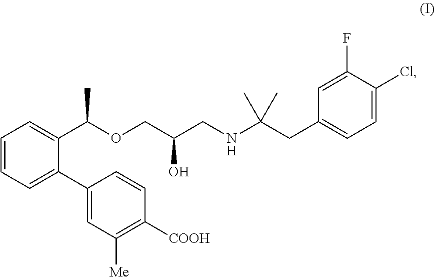 [SMILES: Cc1cc(-c2ccccc2[C@@H](C)OC[C@H](O)CNC(C)(C)Cc2ccc(Cl)c(F)c2)ccc1C(=O)O]

or a solvate, a hydrate, a pharmaceutically acceptable salt, or a combination thereof, according to one or more dosing regimens including a first dosing regimen, a second dosing regimen, and/or a third dosing regimen, wherein:
1) the first dosing regimen includes administering a first therapeutically effective amount of the compound or the solvate, hydrate, pharmaceutically acceptable salt, or combination thereof, wherein the first therapeutically effective amount increases a blood calcium concentration (cCa) up to a maximum cCa of about 10.5 milligrams per deciliter (mg/dL);
2) the second dosing regimen includes administering a second therapeutically effective amount of the compound or the solvate, hydrate, pharmaceutically acceptable salt, or combination thereof, wherein the second therapeutically effective amount titrates the blood calcium concentration (cCa) to a range of about 7.5 mg/dL to about 10.5 mg/dL; and
3) the third dosing regimen includes administering a third therapeutically effective amount of the compound or the solvate, hydrate, pharmaceutically acceptable salt, or combination thereof, wherein the third therapeutically effective amount maintains the blood calcium concentration (cCa) in a range of about 7.5 mg/dL to about 10.5 mg/dL for a period of at least 12 weeks.

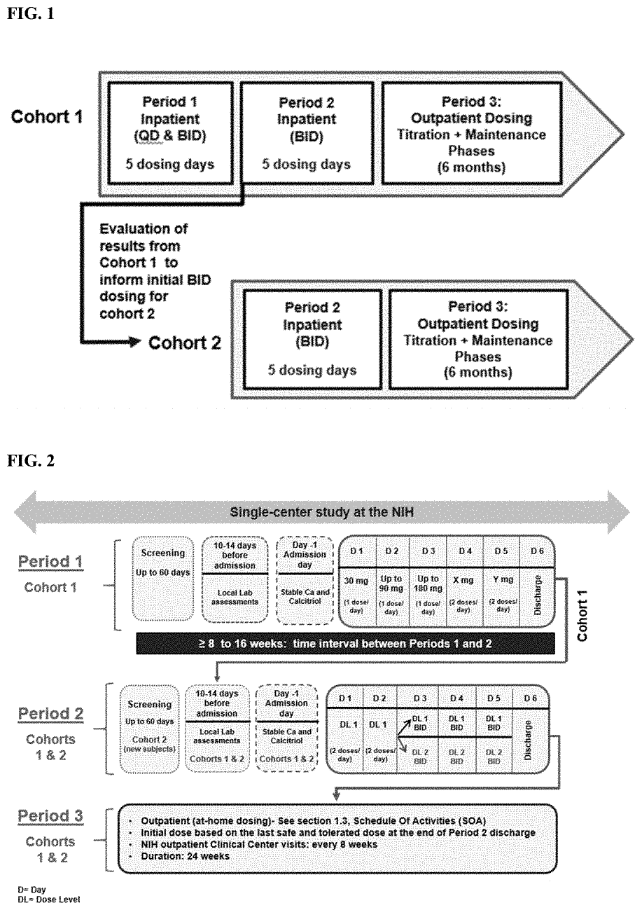

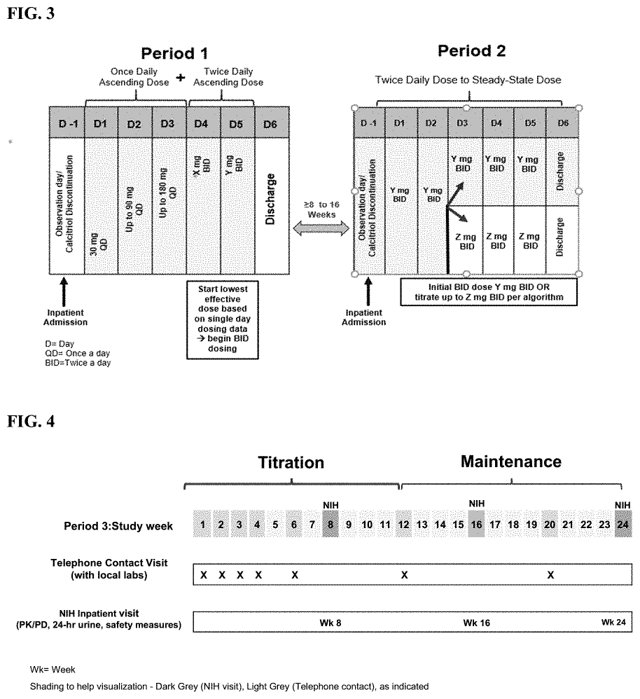

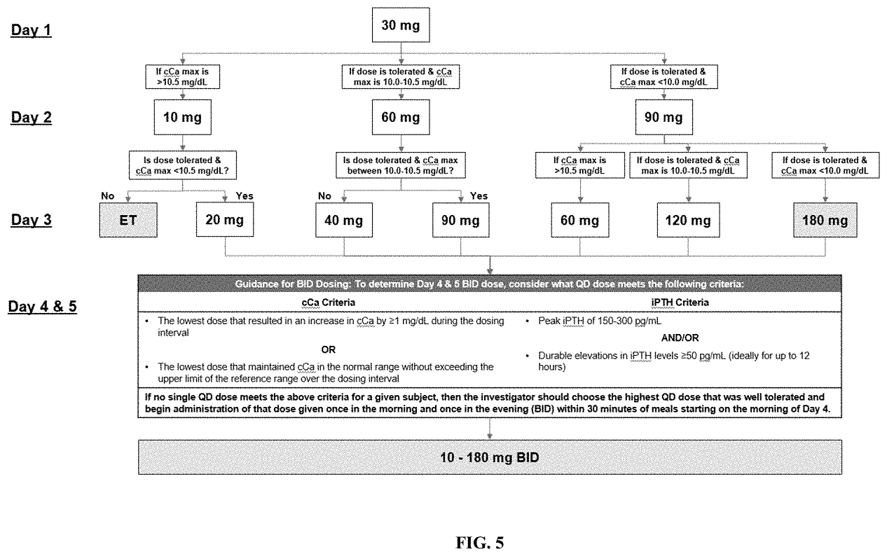

A Phase 2b, open-label dose-ranging study as shown in FIG. 1 and FIG. 2 can evaluate the safety, tolerability, and efficacy of CLTX-305 to maintain normalized albumin-corrected blood calcium (cCa) in subjects with hypocalcemia due to ADH1. The efficacy of the study is described in Section III-4. Study scheme for Periods 1 and 2 (single and multiple ascending dose testing) is detailed in FIG. 3 and study scheme for Period 3 is detailed in FIG. 4.

The compound of formula (I) is described in Section III-1.

In some embodiments, the compound of formula (I) is in a hemihydrate hemisulfate salt form as CLTX-305 represented by the formula:

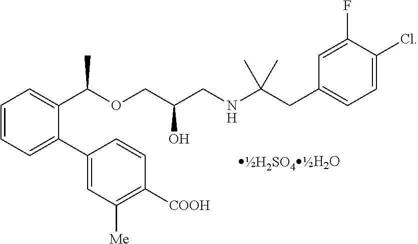 [SMILES: Cc1cc(-c2ccccc2[C@@H](C)OC[C@H](O)CNC(C)(C)Cc2ccc(Cl)c(F)c2)ccc1C(=O)O]

In some embodiments, the compound of formula (I) or CLTX-305 is administered orally. In some embodiments, CLTX-305 is administered orally. In some embodiments, CLTX-305 in a tablet formulation is administered orally.

The subject is described in Section III-2.

In some embodiments, the subject has hypocalcemia, hypoparathyroidism, hypercalciuria, hyperphosphatemia, hypomagnesemia, or a combination thereof. In some embodiments, the subject has hypocalcemia, hypoparathyroidism, hypercalciuria, hyperphosphatemia, or a combination thereof. In some embodiments, the subject has hypocalcemia. In some embodiments, the subject has hypoparathyroidism. In some embodiments, the subject has hyperphosphatemia. In some embodiments, the subject has hypercalciuria. In some embodiments, the subject has hypocalcemia and hypoparathyroidism. In some embodiments, the subject has hypocalcemia and hypercalciuria. In some embodiments, the subject has hypocalcemia, hypoparathyroidism, and hypercalciuria. In some embodiments, the subject has hypocalcemia, hypoparathyroidism, hypercalciuria, and hyperphosphatemia. In some embodiments, the subject has hypocalcemia, hypoparathyroidism, hypercalciuria, hyperphosphatemia, and hypomagnesemia.

In some embodiments, the subject has an activating mutation of the calcium-sensing receptor (CASR) gene.

In some embodiments, prior to the treatment with CLTX-305, the subject does not have a vitamin D deficiency. In some embodiments, the subject has 25-hydroxy-vitamin D in blood at a level of at least about 25 ng/mL. In some embodiments, the subject has 25-hydroxy-vitamin D in blood at a level of from about 25 ng/mL to about 60 ng/mL. In some embodiments, the subject has 25-hydroxy-vitamin D in blood at a level of from about 30 ng/mL to about 60 ng/mL. In some embodiments, the subject has 25-hydroxy-vitamin D in blood at a level of from about 40 ng/mL to about 60 ng/mL.

In some embodiments, the subject is not treated with calcitriol while being treated with CLTX-305. In some embodiments, the subject receives a daily calcium intake of at least about 1000 mg from diet and/or supplementation while being treated with CLTX-305. In some embodiments, the subject receives a daily calcium intake of at least about 1000 mg from diet and supplementation while being treated with CLTX-305.

In some embodiments, the first dosing regimen includes a total daily dosage of at least about 10 mg, 20 mg, 30 mg, 40 mg, 60 mg, 90 mg, 120 mg, 180 mg, or 360 mg of CLTX-305. In some embodiments, the first dosing regimen includes a total daily dosage of at least about 30 mg, 60 mg, 90 mg, 120 mg, 180 mg, or 360 mg of CLTX-305. In some embodiments, the first dosing regimen includes a total daily dosage of about 30 mg, 60 mg, 90 mg, 180 mg, or 360 mg of CLTX-305. In some embodiments, the first dosing regimen includes a total daily dosage of about 30 mg, 90 mg, or 180 mg of CLTX-305.

In general, the first dosing regimen includes an initial total daily dosage of a low amount of CLTX-305 (e.g., about 10-30 mg). In some embodiments, the first dosing regimen includes an initial total daily dosage of about 30 mg of CLTX-305.

In general, during the first dosing regimen, CLTX-305 can be administered once or multiple times (e.g., 2, 3, or 4 times) daily. In some embodiments, the first dosing regimen includes administering CLTX-305 once daily for initial three days and twice daily for two days.

In some embodiments, the first dosing regimen comprises twice daily administration of CLTX-305 to provide a total daily dosage of at least about 10 mg, 20 mg, 30 mg, 40 mg, 60 mg, 90 mg, 120 mg, 180 mg, or 360 mg of CLTX-305. In some embodiments, the first dosing regimen comprises twice daily administration of CLTX-305 to provide a total daily dosage of at least about 30 mg, 60 mg, 90 mg, 120 mg, 180 mg, or 360 mg of CLTX-305. In some embodiments, the first dosing regimen comprises twice daily administration of CLTX-305 to provide a total daily dosage of about 30 mg, 60 mg, 90 mg, 180 mg, or 360 mg of CLTX-305. In some embodiments, the first dosing regimen comprises twice daily administration of CLTX-305 to provide a total daily dosage of about 30 mg, 90 mg, or 180 mg of CLTX-305. In some embodiments, the first dosing regimen comprises a first period of once daily dosing and a second period of twice daily dosing. In some embodiments, the first period of once daily dosing precedes the second period of twice daily dosing. In some embodiments, the total daily dosage in the first period is the same as the total daily dosage in the second period. In some embodiments, the total daily dosage in the first period differs from the total daily dosage in the second period.

The first dosing regimen can include one or more dose adjustments according to a level of blood calcium concentration (cCa) in a subject, for example increasing or decreasing the total daily dosage whether the subject reaches a maximum cCa of about 10.5 mg/dL, or increasing the daily dosing frequency (e.g., from once daily to twice daily) while maintaining the total daily dosage, when the subject reaches the maximum cCa of about 10.5 mg/dL. In some embodiments, the first dosage includes:
a1) increasing the total daily dosage, when the blood calcium concentration (cCa) is less than a maximum cCa of about 10.5 mg/dL;
b1) decreasing the total daily dosage, when the blood calcium concentration (cCa) reaches the maximum cCa; and/or
c1) increasing the daily dosing frequency while maintaining the total daily dosage, when the blood calcium concentration (cCa) reaches the maximum cCa.

The blood calcium concentration (cCa) in the subject is determined after each of one or more once-daily dosages and one or more twice-daily dosages in order to determine whether one or more dose adjustments are to be made. In some embodiments, the blood calcium concentration (cCa) is determined after each of one or more once-daily dosages and one or more twice-daily dosages. In some embodiments, the blood calcium concentration (cCa) is determined after the once-daily dosage on day−1, day−2, and day−3 and the twice-daily dosage on day−4 and day−5.

When the blood calcium concentration (cCa) is less than the maximum cCa of about 10.5 mg/dL, the total daily dosage is increased. The total daily dosage can be increased per algorithm according to parameters of the subject (e.g., blood cCa value, a net increased cCa over a dosing interval, peak iPTH, and/or a durable elevation of iPTH). In some embodiments, the total daily dosage is increased according to algorithm as shown in FIG. 5. In some embodiments, the total daily dosage is increased by doubling a prior total daily dosage (e.g., from 30 mg to 60 mg, from 60 mg to 120 mg, from 90 mg to 180 mg, and so on). In some embodiments, the total daily dosage is increased by tripling a prior total daily dosage (e.g., from 10 mg to 30 mg, from 30 mg to 90 mg, from 60 mg to 180 mg, from 90 mg to 270 mg, and so on). In some embodiments, the total daily dosage is increased from about 30 mg to about 60 mg, from about 30 mg to about 90 mg, from about 30 mg to about 120 mg, from about 30 mg to about 180 mg, from about 10 mg to about 20 mg, from about 20 mg to about 40 mg, from about 60 mg to about 90 mg, from about 60 mg to about 120 mg, from about 90 mg to about 120 mg, from about 90 mg to about 180 mg, or from about 180 mg to about 360 mg of CLTX-305. In some embodiments, the total daily dosage is increased from about 30 mg to about 60 mg, from about 30 mg to about 90 mg, from about 30 mg to about 120 mg, from about 30 mg to about 180 mg, from about 10 mg to about 20 mg, from about 20 mg to about 40 mg, from about 60 mg to about 90 mg, from about 60 mg to about 120 mg, from about 90 mg to about 120 mg, or from about 90 mg to about 180 mg of CLTX-305. In some embodiments, the total daily dosage is increased from about 30 mg to about 60 mg, from about 30 mg to about 90 mg, from about 90 mg to about 120 mg, or from about 90 mg to about 180 mg of CLTX-305.

When the blood calcium concentration (cCa) reaches the maximum cCa of about 10.5 mg/dL, the total daily dosage is decreased. The total daily dosage can be decreased per algorithm according to parameters of the subject (e.g., blood cCa value, a net increased cCa over a dosing interval, peak iPTH, and/or a durable elevation of iPTH). In some embodiments, the total daily dosage is decreased according to algorithm as shown in FIG. 5. In some embodiments, the total daily dosage is decreased by two-third (⅔) of a prior total daily dosage (e.g., from 60 mg to 40 mg, from 90 mg to 60 mg, from 180 mg to 120 mg, and so on). In some embodiments, the total daily dosage is decreased by a half (½) of a prior total daily dosage (e.g., from 60 mg to 30 mg, from 120 mg to 60 mg, from 180 mg to 90 mg, and so on). In some embodiments, the total daily dosage is decreased by one-third (⅓) of a prior total daily dosage (e.g., from 30 mg to 10 mg, from 90 mg to 30 mg, from 180 mg to 60 mg, from 270 mg to 90 mg, and so on). In some embodiments, the total daily dosage is decreased from about 30 mg to about 10 mg, from about 40 mg to about 20 mg, from about 60 mg to about 40 mg, from about 90 mg to about 60 mg, from about 90 mg to about 30 mg, from about 120 mg to about 90 mg, from about 120 mg to about 60 mg, from about 180 mg to about 120 mg, from about 180 mg to about 90 mg, from about 180 mg to about 60 mg, or from about 360 mg to about 180 mg of CLTX-305. In some embodiments, the total daily dosage is decreased from about 30 mg to about 10 mg, from about 60 mg to about 40 mg, from about 90 mg to about 60 mg, from about 90 mg to about 30 mg, from about 180 mg to about 120 mg, from about 180 mg to about 90 mg, or from about 180 mg to about 60 mg of CLTX-305.

When the blood calcium concentration (cCa) reaches the maximum cCa of about 10.5 mg/dL, the daily dosing frequency is increased while maintaining the total daily dosage of CLTX-305. In some embodiments, the daily dosing frequency is increased from once to twice daily while maintaining the total daily dosage of CLTX-305. In some embodiments, the daily dosing frequency is increased from once to twice daily while maintaining the total daily dosage of about 30 mg, 60 mg, 90 mg, 120 mg, 180 mg, or 360 mg of CLTX-305. In some embodiments, the daily dosing frequency is increased from once to twice daily while maintaining the total daily dosage of about 30 mg, 60 mg, 90 mg, 120 mg, or 180 mg of CLTX-305.

In some embodiments, the first dosing regimen further includes:
i) selecting a lowest dosage among one or more once-daily dosages and administering the lowest dosage twice daily, provided that the one or more once-daily dosages meet a criteria selected form the group consisting of:
   i-1) the blood calcium concentration (cCa) is increased by at least about 1 mg/dL over a dosing interval;
   i-2) the blood calcium concentration (cCa) is maintained in a range of about 7.5 to about 10.5 mg/dL;
   i-3) intact parathyroid hormone (iPTH) in blood is increased to a peak level of about 150 to about 300 pg/mL; and
   i-4) intact parathyroid hormone (iPTH) in blood is increased to an elevated level of at least about 50 pg/mL and maintained for a period of about 12 hours,
      or
ii) selecting a highest dosage among one or more once-daily dosages and administering the highest dosage twice daily, provided that the one or more once-daily dosages do not meet any one of criteria i-1) to i-4); and the highest dosage is tolerable in the subject.

In some embodiments, the lowest dosage among one or more once-daily dosages for twice daily dosing is about 30 mg, 60 mg, 90 mg, 120 mg, or 180 mg of CLTX-305, provided the one or more once-daily dosages meet any one of criteria i-1) to i-4). In some embodiments, the lowest dosage among one or more once-daily dosages for twice daily dosing is about 90 mg or 180 mg of CLTX-305, provided the one or more once-daily dosages meet any one of criteria i-1) to i-4).

In some embodiments, the highest dosage among one or more once-daily dosages for twice daily dosing is about 30 mg, 60 mg, 90 mg, 120 mg, or 180 mg of CLTX-305, provided the one or more once-daily dosages do not meet any one of criteria i-1) to i-4). In some embodiments, the highest dosage among one or more once-daily dosages for twice daily dosing is about 90 mg or 180 mg of CLTX-305, provided the one or more once-daily dosages do not meet any one of criteria i-1) to i-4).

During or at the end of the first dosing regimen, the subject is evaluated via both blood and urine sampling. The blood and urine sampling schedules are described in Section III-4. In some embodiments, blood and urine sampling are scheduled according to Table 4A-Table 4C, Table 5, Table 6A-Table 6C, and Table 7 of Example 1.

In some embodiments, the second dosing regimen includes a total daily dosage of at least about 10 mg, 20 mg, 30 mg, 40 mg, 60 mg, 90 mg, 120 mg, 180 mg, 360 mg, 480 mg, or 720 mg of CLTX-305. In some embodiments, the second dosing regimen includes a total daily dosage of at least about 10 mg, 20 mg, 40 mg, 60 mg, 120 mg, 180 mg, 360 mg, 480 mg, or 720 mg of CLTX-305. In some embodiments, the second dosing regimen includes a total daily dosage of at least about 60 mg, 120 mg, 180 mg, or 360 mg of CLTX-305. In some embodiments, the second dosing regimen includes a total daily dosage of at least about 30 mg, 90 mg, 120 mg, or 180 mg of CLTX-305. In some embodiments, the second dosing regimen includes a total daily dosage of at least about 30 mg, 90 mg, or 180 mg of CLTX-305. In some embodiments, the second dosing regimen includes a total daily dosage of about 40 mg, 60 mg, 120 mg, 180 mg, 360 mg, 480 mg, or 720 mg of CLTX-305. In some embodiments, the second dosing regimen includes a total daily dosage of about 60 mg, 120 mg, 180 mg, 360 mg, 480 mg, or 720 mg of CLTX-305. In some embodiments, the second dosing regimen includes a total daily dosage of about 60 mg, 120 mg, 180 mg, 360 mg, 480 mg, or 720 mg of CLTX-305. In some embodiments, the second dosing regimen includes a total daily dosage of about 180 mg or 360 mg of CLTX-305.

In general, the second dosing regimen includes an initial total daily dosage of CLTX-305 calculated based on the total daily dosage on the last day of the first dosing regimen (e.g., about 10 mg, 30 mg, 60 mg, 90 mg, 120 mg, 180 mg, or 360 mg). In some embodiments, the second dosing regimen includes an initial total daily dosage of about 10 mg, 30 mg, 60 mg, 90 mg, 120 mg, 180 mg, 360 mg, 480 mg, or 720 mg of CLTX-305. In some embodiments, the second dosing regimen includes an initial total daily dosage of about 60 mg, 90 mg, 120 mg, 180 mg, 360 mg, or 480 mg of CLTX-305. In some embodiments, the second dosing regimen includes an initial total daily dosage of about 60 mg, 90 mg, 120 mg, 180 mg, or 360 mg of CLTX-305.

In general, during the second dosing regimen, CLTX-305 can be administered twice or multiple times (e.g., 2, 3, or 4 times) daily. In some embodiments, the second dosing regimen includes administering CLTX-305 in a daily dosing frequency of 2 to 4 times daily. In some embodiments, the second dosing regimen includes administering CLTX-305 twice daily. In some embodiments, the second dosing regimen includes administering CLTX-305 twice daily for five days.

In some embodiments, the second dosing regimen comprises twice daily administration of CLTX-305 to provide a total daily dosage of at least about 10 mg, 20 mg, 30 mg, 40 mg, 60 mg, 90 mg, 120 mg, 180 mg, or 360 mg of CLTX-305. In some embodiments, the second dosing regimen comprises twice daily administration of CLTX-305 to provide a total daily dosage of at least about 30 mg, 60 mg, 90 mg, 120 mg, 180 mg, or 360 mg of CLTX-305. In some embodiments, the second dosing regimen comprises twice daily administration of CLTX-305 to provide a total daily dosage of about 30 mg, 60 mg, 90 mg, 180 mg, or 360 mg of CLTX-305. In some embodiments, the second dosing regimen comprises twice daily administration of CLTX-305 to provide a total daily dosage of about 30 mg, 90 mg, or 180 mg of CLTX-305. In some embodiments, the second dosing regimen comprises a first period of once daily dosing and a second period of twice daily dosing. In some embodiments, the first period of once daily dosing precedes the second period of twice daily dosing. In some embodiments, the total daily dosage in the first period is the same as the total daily dosage in the second period. In some embodiments, the total daily dosage in the first period differs from the total daily dosage in the second period.

The second dosing regimen can include one or more dose adjustments or no dose adjustments according to a level of blood calcium concentration (cCa) in a subject, for example increasing or decreasing the total daily dosage if the blood calcium concentration (cCa) is not in a range of from about 7.5 to about 10.5 mg/dL, or maintaining the total daily dosage when the blood calcium concentration (cCa) is in the prescribed range. In some embodiments, the second dosing regimen includes:
a2) maintaining the total daily dosage, when the blood calcium concentration (cCa) is maintained in a range of from about 7.5 to about 10.5 mg/dL;
b2) increasing the total daily dosage, when the blood calcium concentration (cCa) is less than about 7.5 mg/dL; or
c2) decreasing the total daily dosage when the blood calcium concentration (cCa) is more than about 10.5 mg/dL.

The blood calcium concentration (cCa) in the subject is determined after the first dosing regimen, prior to the second dosing regimen, or during the second dosing regimen. In some embodiments, the blood calcium concentration (cCa) is determined after the first dosing regimen, prior to the second dosing regimen, or during the second dosing regimen. In some embodiments, the blood calcium concentration (cCa) is determined after the first dosing regimen or after initial two days of the second dosing regimen.

When the blood calcium concentration (cCa) is maintained in a range of from about 7.5 to about 10.5 mg/dL, the total daily dosage of CLTX-305 (e.g., an initial total daily dosage or an adjusted total daily dosage) can be maintained. The initial total daily dosage can be the total daily dosage on Day-1 and Day-2. The adjusted total daily dosage can be the total daily dosage on Day-3, Day-4, and/or Day-5. In some embodiments, the second dosing regimen maintains the total daily dosage of CLTX-305, when the blood calcium concentration (cCa) is maintained in a range of from about 7.5 to about 10.5 mg/dL. In some embodiments, the second dosing regimen maintains an initial total daily dosage of CLTX-305, when the blood calcium concentration (cCa) is maintained in a range of from about 7.5 to about 10.5 mg/dL. In some embodiments, the second dosing regimen maintains an adjusted total daily dosage of CLTX-305, when the blood calcium concentration (cCa) is maintained in a range of from about 7.5 to about 10.5 mg/dL. In some embodiments, the second dosing regimen maintains an initial total daily dosage of CLTX-305 on Day-1 and Day-2, when the blood calcium concentration (cCa) is maintained in a range of from about 7.5 to about 10.5 mg/dL. In some embodiments, the second dosing regimen maintains an adjusted total daily dosage of CLTX-305 on Day-3, Day-4, and/or Day-5, when the blood calcium concentration (cCa) is maintained in a range of from about 7.5 to about 10.5 mg/dL.

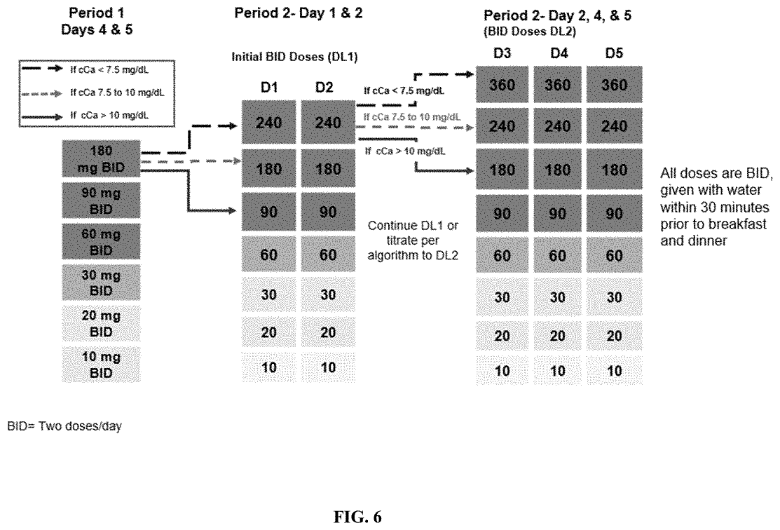

When the blood calcium concentration (cCa) is less than about 7.5 mg/dL, the total daily dosage is increased. The total daily dosage can be increased per algorithm according to parameters of the subject (e.g., blood cCa value, a net increased cCa over a dosing interval, peak iPTH, and/or a durable elevation of iPTH). In some embodiments, the total daily dosage is increased according to algorithm as shown in FIG. 6. In some embodiments, the total daily dosage is increased by doubling a prior total daily dosage (e.g., from 30 mg to 60 mg, from 60 mg to 120 mg, from 90 mg to 180 mg, and so on). In some embodiments, the total daily dosage is increased by tripling a prior total daily dosage (e.g., from 10 mg to 30 mg, from 30 mg to 90 mg, from 60 mg to 180 mg, from 90 mg to 270 mg, and so on). In some embodiments, the total daily dosage is increased from about 20 mg to about 40 mg, from about 40 mg to about 60 mg, from about 60 mg to about 120 mg, from about 120 mg to about 180 mg, from about 180 mg to about 360 mg, from about 360 mg to about 480 mg, or from about 480 mg to about 720 mg of CLTX-305. In some embodiments, the total daily dosage is increased from about 30 mg to about 60 mg, from about 30 mg to about 90 mg, from about 10 mg to about 20 mg, from about 20 mg to about 40 mg, from about 60 mg to about 120 mg, from about 90 mg to about 180 mg, or from about 180 mg to about 360 mg of CLTX-305. In some embodiments, the total daily dosage is increased from about 30 mg to about 90 mg, from about 60 mg to about 120 mg, from about 90 mg to about 180 mg, or from about 180 mg to about 360 mg of CLTX-305. In some embodiments, the total daily dosage is increased from about 30 mg to about 90 mg, from about 90 mg to about 180 mg, or from about 180 mg to about 360 mg of CLTX-305. In some embodiments, the total daily dosage is increased from about 30 mg to about 90 mg or from about 90 mg to about 180 mg of CLTX-305.

When the blood calcium concentration (cCa) is more than about 10.5 mg/dL, the total daily dosage is decreased. The total daily dosage can be decreased per algorithm according to parameters of the subject (e.g., blood cCa value, a net increased cCa over a dosing interval, peak iPTH, and/or a durable elevation of iPTH). In some embodiments, the total daily dosage is decreased according to algorithm as shown in FIG. 6. In some embodiments, the total daily dosage is decreased by two-third (⅔) of a prior total daily dosage (e.g., from 60 mg to 40 mg, from 90 mg to 60 mg, from 180 mg to 120 mg, and so on). In some embodiments, the total daily dosage is decreased by a half (½) of a prior total daily dosage (e.g., from 60 mg to 30 mg, from 120 mg to 60 mg, from 180 mg to 90 mg, and so on). In some embodiments, the total daily dosage is decreased by one-third (⅓) of a prior total daily dosage (e.g., from 30 mg to 10 mg, from 90 mg to 30 mg, from 180 mg to 60 mg, from 270 mg to 90 mg, and so on). In some embodiments, the total daily dosage is decreased from about 40 mg to about 20 mg, from about 60 mg to about 40 mg, from about 120 mg to about 60 mg, from about 180 mg to about 120 mg, from about 360 mg to about 180 mg, or from about 480 mg to about 360 mg of CLTX-305. In some embodiments, the total daily dosage is decreased from about 30 mg to about 10 mg, from about 40 mg to about 20 mg, from about 60 mg to about 30 mg, from about 90 mg to about 60 mg, from about 90 mg to about 30 mg, from about 120 mg to about 90 mg, from about 120 mg to about 60 mg, from about 180 mg to about 120 mg, from about 180 mg to about 90 mg, from about 180 mg to about 60 mg, or from about 360 mg to about 180 mg of CLTX-305. In some embodiments, the total daily dosage is decreased from about 30 mg to about 10 mg, from about 90 mg to about 60 mg, from about 90 mg to about 30 mg, from about 180 mg to about 120 mg, from about 180 mg to about 90 mg, from about 180 mg to about 60 mg, or from about 360 mg to about 180 mg of CLTX-305.

During or at the end of the second dosing regimen, the subject is evaluated via both blood and urine sampling. The blood and urine sampling schedules are described in Section III-4. In some embodiments, blood and urine sampling are scheduled according to Table 4A-Table 4C, Table 5, Table 6A-Table 6C, and Table 7 of Example 1.

In some embodiments, the third dosing regimen includes a total daily dosage of at least about 10 mg, 20 mg, 30 mg, 40 mg, 60 mg, 80 mg, 90 mg, 120 mg, 140 mg, 180 mg, 300 mg, 360 mg, 480 mg, or 720 mg of CLTX-305. In some embodiments, the third dosing regimen includes a total daily dosage of at least about 20 mg, 40 mg, 60 mg, 120 mg, 180 mg, 360 mg, 480 mg, or 720 mg of CLTX-305. In some embodiments, the third dosing regimen includes a total daily dosage of at least about 60 mg, 120 mg, 180 mg, or 360 mg of CLTX-305. In some embodiments, the third dosing regimen includes a total daily dosage of at least about 30 mg, 90 mg, or 180 mg of CLTX-305. In some embodiments, the third dosing regimen includes a total daily dosage of about 20 mg, 40 mg, 60 mg, 120 mg, 180 mg, 360 mg, 480 mg, or 720 mg of CLTX-305. In some embodiments, the third dosing regimen includes a total daily dosage of about 60 mg, 120 mg, 180 mg, 360 mg, 480 mg, or 720 mg of CLTX-305. In some embodiments, the third dosing regimen includes a total daily dosage of about 60 mg, 120 mg, 180 mg, 360 mg, 480 mg, or 720 mg of CLTX-305. In some embodiments, the third dosing regimen includes a total daily dosage of about 10 mg, 20 mg, 80 mg, 140 mg, or 300 mg of CLTX-305.

In general, the third dosing regimen includes an initial total daily dosage of CLTX-305 calculated based on a tolerable and/or an effective total daily dosage determined during the second dosing regimen. In some embodiments, the third dosing regimen includes an initial total daily dosage of about 10 mg, 20 mg, 30 mg, 60 mg, 80 mg, 90 mg, 120 mg, 140 mg, 180 mg, 300 mg, 360 mg, 480 mg, or 720 mg of CLTX-305. In some embodiments, the second dosing regimen includes an initial total daily dosage of about 60 mg, 90 mg, 120 mg, 180 mg, 360 mg, or 480 mg of CLTX-305. In some embodiments, the second dosing regimen includes an initial total daily dosage of about 60 mg, 90 mg, 180 mg, or 360 mg of CLTX-305.

In general, during the third dosing regimen, CLTX-305 can be administered twice or multiple times (e.g., 2, 3, or 4 times) daily. In some embodiments, the third dosing regimen includes administering CLTX-305 in a daily dosing frequency of 2 to 4 times daily. In some embodiments, the third dosing regimen includes administering CLTX-305 twice daily. In some embodiments, the third dosing regimen includes administering CLTX-305 twice daily for at least 24 weeks.

In some embodiments, the third dosing regimen comprises twice daily administration of CLTX-305 to provide a total daily dosage of at least about 10 mg, 20 mg, 30 mg, 40 mg, 60 mg, 80 mg, 90 mg, 120 mg, 140 mg, 180 mg, 300 mg, 360 mg, 480 mg, or 720 mg of CLTX-305. In some embodiments, the third dosing regimen comprises twice daily administration of CLTX-305 to provide a total daily dosage of at least about 30 mg, 60 mg, 90 mg, 120 mg, 180 mg, or 360 mg of CLTX-305. In some embodiments, the third dosing regimen comprises twice daily administration of CLTX-305 to provide a total daily dosage of about 30 mg, 60 mg, 90 mg, 180 mg, or 360 mg of CLTX-305. In some embodiments, the third dosing regimen comprises twice daily administration of CLTX-305 to provide a total daily dosage of about 30 mg, 90 mg, or 180 mg of CLTX-305. In some embodiments, the third dosing regimen comprises a first period of once daily dosing and a second period of twice daily dosing. In some embodiments, the first period of once daily dosing precedes the second period of twice daily dosing. In some embodiments, the total daily dosage in the first period is the same as the total daily dosage in the second period. In some embodiments, the total daily dosage in the first period differs from the total daily dosage in the second period.

The third dosing regimen can include a titration period and a maintenance period, each of which can last up to 12 weeks. The titration period involves one or more dose adjustments or no dose adjustments in order to have the blood calcium concentration (cCa) in a normal to low-normal range (e.g., about 7.5 to about 10.5 mg/dL). The maintenance period is to maintain a stable dose, a stable normal to low-normal cCa while avoiding hypercalciuria in the subject. In some embodiments, the third dosing regimen includes a titration period. In some embodiments, the third dosing regimen includes titrating the total daily dosage while maintaining the blood calcium concentration (cCa) in a range of from about 7.5 to about 10.5 mg/dL. In some embodiments, the third dosing regimen includes titrating the total daily dosage while maintaining the blood calcium concentration (cCa) in a range of from about 8.5 to about 10.5 mg/dL. In some embodiments, the third dosing regimen includes a maintenance period. In some embodiments, the third dosing regimen includes maintaining the total daily dosage while maintaining the blood calcium concentration (cCa) in a range of from about 7.5 to about 10.5 mg/dL. In some embodiments, the third dosing regimen includes maintaining the total daily dosage while maintaining the blood calcium concentration (cCa) in a range of from about 8.5 to about 10.5 mg/dL. In some embodiments, the third dosing regimen includes maintaining the total daily dosage while maintaining the blood calcium concentration (cCa) in a range of from about 7.5 to about 10.5 mg/dL and minimizes hypercalciuria in the subject. In some embodiments, the third dosing regimen includes maintaining the total daily dosage while maintaining the blood calcium concentration (cCa) in a range of from about 8.5 to about 10.5 mg/dL and minimizes hypercalciuria in the subject.

The third dosing regimen (e.g., the titration period) can include one or more dose adjustments or no dose adjustments according to a level of blood calcium concentration (cCa) in a subject, for example increasing or decreasing the total daily dosage if the blood calcium concentration (cCa) is not in a range of from about 7.5 to about 10.5 mg/dL, or maintaining the total daily dosage when the blood calcium concentration (cCa) is in the prescribed range. In some embodiments, the third dosing regimen includes:
a3) maintaining the total daily dosage, when the blood calcium concentration (cCa) is maintained in a range of from about 7.5 to about 10.5 mg/dL;
b3) increasing the total daily dosage, when the blood calcium concentration (cCa) is less than about 7.5 mg/dL; or
c3) decreasing the total daily dosage when the blood calcium concentration (cCa) is more than about 10.5 mg/dL.

The blood calcium concentration (cCa) in the subject is determined after the second dosing regimen, prior to the third dosing regimen, or during the third dosing regimen. In some embodiments, the blood calcium concentration (cCa) is determined after the second dosing regimen, prior to the third dosing regimen, or during the third dosing regimen. In some embodiments, the blood calcium concentration (cCa) is determined prior to the third dosing regimen, or during the third dosing regimen. In some embodiments, the blood calcium concentration (cCa) is determined during the third dosing regimen. In some embodiments, the blood calcium concentration (cCa) is determined during the third dosing regimen during weeks 1-4, week-6, week-8, week-12, week-16, week-20, and/or week-24 according to FIG. 4.

When the blood calcium concentration (cCa) is maintained in a range of from about 7.5 to about 10.5 mg/dL, the total daily dosage of CLTX-305 (e.g., an initial total daily dosage or an adjusted total daily dosage) can be maintained during the titration period. In some embodiments, the third dosing regimen maintains the total daily dosage of CLTX-305 during the titration period, when the blood calcium concentration (cCa) is maintained in a range of from about 7.5 to about 10.5 mg/dL. In some embodiments, the third dosing regimen maintains an initial total daily dosage of CLTX-305 during the titration period, when the blood calcium concentration (cCa) is maintained in a range of from about 7.5 to about 10.5 mg/dL. In some embodiments, the third dosing regimen maintains an adjusted total daily dosage of CLTX-305 during the titration period, when the blood calcium concentration (cCa) is maintained in a range of from about 7.5 to about 10.5 mg/dL.

When the blood calcium concentration (cCa) is less than about 7.5 mg/dL, the total daily dosage is increased during the titration period. The total daily dosage can be increased per algorithm according to parameters of the subject (e.g., blood cCa value, body weight, age, sex, and/or other conditions). In some embodiments, the total daily dosage during the titration period is increased according to algorithm.

When the blood calcium concentration (cCa) is more than about 10.5 mg/dL, the total daily dosage is decreased during the titration period. The total daily dosage can be decreased per algorithm according to parameters of the subject (e.g., blood cCa value, body weight, age, sex, and/or other conditions). In some embodiments, the total daily dosage during the titration period is decreased according to algorithm.

During the third dosing regimen, a titration period can extend up to 12 weeks, after which each individualized dose regimen continues to be administered with a goal to maintain a stable dose, a stable normal to low-normal cCa while avoiding hypercalciuria in the subject. The need for additional dose adjustments during the maintenance period may depend on factors potentially related to the time course of changes in parathyroid gland function, intestinal calcium absorption, bone resorption, and kidney function, all of which are monitored. In some embodiments, the third dosing regimen during the maintenance period includes one or more one or more dose adjustments according to one or more parameters of parathyroid gland function, intestinal calcium absorption, bone resorption, and kidney function.

The goals of the third dosing regimen are to reduce symptoms associated with hypocalcemia and hypercalcemia, and to minimize hypercalciuria in the subject. In some embodiments, the third dosing regimen reduces symptoms associated with hypocalcemia and hypercalcemia, and minimizes hypercalciuria in the subject. In some embodiments, the third dosing regimen maintains the blood calcium concentration (cCa) in a range of about 7.5 to about 10.5 mg/dL for a period of at least 12 weeks. In some embodiments, the third dosing regimen maintains the blood calcium concentration (cCa) in a range of about 7.5 to about 10.5 mg/dL for a period of at least 24 weeks. In some embodiments, the third dosing regimen maintains the blood calcium concentration (cCa) in a range of about 8.5 to about 10.5 mg/dL for a period of at least 12 weeks. In some embodiments, the third dosing regimen maintains the blood calcium concentration (cCa) in a range of about 8.5 to about 10.5 mg/dL for a period of at least 24 weeks.

During the third dosing regimen, the subject is not on calcitriol but may take additional calcium supplementation as needed if a minimum daily dietary intake of about 1000 mg cannot be achieved and the subject has persistent hypocalcemia. Titration of oral calcium supplements can be directed during the third dosing regimen and may be up or down titrated based on achieving the therapeutic goal of a normal or low-normal cCa without hypercalciuria. In some embodiments, the third dosing regimen further includes administering an oral calcium supplementation in addition to the daily dietary calcium intake.

During or at the end of the third dosing regimen, the subject is for both blood and urine sampling. The blood and urine sampling schedules are described in Section III-4 and noted weeks as shown in FIG. 4. In some embodiments, blood and urine sampling are scheduled according to Table 4A-Table 4C, Table 5, Table 6A-Table 6C, and Table 7 of Example 1.

Prior to, during, and/or after any one of the first, second, and/or third dosing period, the subject can be evaluated by one or more tests including blood analyses, urine analyses, and/or hematology tests. Examples of such tests are described in Section III-4, Table 1, Table 2, and Table 3 of Example 1.

In some embodiments, the blood analyses include analyses of iPTH, serum bone markers, 1, 25-hydroxy-vitamin D, cAMP, phosphate (P), magnesium (Mg), potassium (K), albumin, creatinine (Cr), and/or creatine kinase (CK). In some embodiments, serum bone markers are blood collagen cross-linked C-telopeptide (CTx) and blood procollagen type 1 N-propeptide (P1NP).

In some embodiments, the blood analyses include analyses of electrolytes (sodium, potassium, chloride), calcium, magnesium, phosphorus, bicarbonate, glucose, blood urea nitrogen (BUN), creatinine, ALT, AST, alkaline phosphatase (ALP), LDH, amylase, lipase, uric acid, creatine kinase (CK), total protein, albumin, total bilirubin, intact PTH, 25-hydroxy vitamin D, 1, 25-dihydroxy-vitamin D, CTx, and/or P1NP.

In some embodiments, the urine analyses include analyses of phosphate (P), magnesium (Mg), sodium (Na), potassium (K), creatinine (Cr), cAMP, citrate, and/or a pH value.

In some embodiments, the urine analyses include urinalysis including a gravity, a pH, protein, glucose, ketones, and/or blood, and analyses of cAMP, calcium, creatinine, magnesium, phosphorus, and/or citrate.

In some embodiments, the hematology tests include hemoglobin, hematocrit, complete blood count (CBC) including red cell count and indices, HCT, Hgb, white blood cell (WBC) count, platelet count, and differential; and coagulation tests including PT, PTT, and/or INR.

### III-6: Oral Dosage Form

The oral dosage form including the compound of formula (I) or CLTX-305 can be in any oral dosage forms including one or more pharmaceutically acceptable carriers and/or excipients. Oral preparations include tablets, pills, powder, dragees, capsules, liquids, lozenges, cachets, gels, syrups, slurries, suspensions, etc., suitable for ingestion by the patient.

For preparing oral dosage forms including the compound of formula (I) or CLTX-305, pharmaceutically acceptable carriers can be either solid or liquid. Solid form preparations include powders, tablets, pills, capsules, cachets, suppositories, and dispersible granules. A solid carrier can be one or more substances, which may also act as diluents, flavoring agents, binders, preservatives, tablet disintegrating agents, or an encapsulating material. Details on techniques for formulation and administration are well described in the scientific and patent literature, see, e.g., the latest edition of Remington's Pharmaceutical Sciences, Maack Publishing Co, Easton Pa. (“Remington's”).

In powders, the carrier is a finely divided solid, which is in a mixture with the finely divided active component. In tablets, the active component is mixed with the carrier having the necessary binding properties in suitable proportions and compacted in the shape and size desired.

The powders, capsules and tablets preferably contain from 5% or 10% to 70% of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as a carrier providing a capsule in which the active component with or without other excipients, is surrounded by a carrier, which is thus in association with it. Similarly, cachets and lozenges are included. Tablets, powders, capsules, pills, cachets, and lozenges can be used as solid dosage forms suitable for oral administration.

Suitable solid excipients include, but are not limited to, magnesium carbonate; magnesium stearate; talc; pectin; dextrin; starch; tragacanth; a low melting wax; cocoa butter; carbohydrates; sugars including, but not limited to, lactose, sucrose, mannitol, or sorbitol, starch from corn, wheat, rice, potato, or other plants; cellulose such as methyl cellulose, hydroxypropylmethyl-cellulose, or sodium carboxymethylcellulose; and gums including arabic and tragacanth; as well as proteins including, but not limited to, gelatin and collagen. If desired, disintegrating or solubilizing agents may be added, such as the cross-linked polyvinyl pyrrolidone, agar, alginic acid, or a salt thereof, such as sodium alginate.

Dragee cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound (i.e., dosage). Pharmaceutical preparations of the dosage forms can also be used orally using, for example, push-fit capsules made of gelatin, as well as soft, sealed capsules made of gelatin and a coating such as glycerol or sorbitol. Push-fit capsules can contain the compound of formula (I) or CLTX-305 mixed with a filler or binders such as lactose or starches, lubricants such as talc or magnesium stearate, and, optionally, stabilizers. In soft capsules, the compound of formula (I) or CLTX-305 may be dissolved or suspended in suitable liquids, such as fatty oils, liquid paraffin, or liquid polyethylene glycol with or without stabilizers.

For preparing suppositories, a low melting wax, such as a mixture of fatty acid glycerides or cocoa butter, is first melted and the compound of formula (I) or CLTX-305 are dispersed homogeneously therein, as by stirring. The molten homogeneous mixture is then poured into convenient sized molds, allowed to cool, and thereby to solidify.

Liquid form preparations include solutions, suspensions, and emulsions, for example, water or water/propylene glycol solutions.

Aqueous solutions suitable for oral use can be prepared by dissolving the compound of formula (I) or CLTX-305 in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity.

Also included are solid form preparations, which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

Oil suspensions can be formulated by suspending the compound of formula (I) or CLTX-305 in a vegetable oil, such as arachis oil, olive oil, sesame oil or coconut oil, or in a mineral oil such as liquid paraffin; or a mixture of these. The oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto, J. Pharmacol. Exp. Ther. 281:93-102, 1997. The pharmaceutical formulations including the compound of formula (I) or CLTX-305 can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent.

The oral dosage form including the compound of formula (I) or CLTX-305 can be any one of tablet formulations as disclosed in International Patent Application No. PCT/US2021/044295 filed Aug. 3, 20221, which is incorporated herein in its entirety for all purposes.

In some embodiments, the oral dosage form is a tablet formulation as described in International Patent Application No. PCT/US2021/044295. The tablet formulation includes:
a) the compound of formula (I), a solvate, a hydrate, a pharmaceutically acceptable salt, or a combination thereof; and
b) one or more pharmaceutically acceptable excipients selected from one or more fillers, one or more glidants, one or more disintegrants, one or more surfactants, one or more binders, one or more lubricants, and a combination thereof,
   wherein the compound is present in an amount of at least about 12% by weight, on a salt-free and anhydrous basis.

In some embodiments, the oral dosage form is a tablet formulation as a common blend formulation across all dosage strengths as described in International Patent Application No. PCT/US2021/044295. The common blend tablet formulation includes:
a) CLTX-305 represented by the formula:

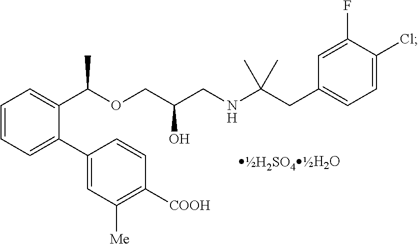 [SMILES: Cc1cc(-c2ccccc2[C@@H](C)OC[C@H](O)CNC(C)(C)Cc2ccc(Cl)c(F)c2)ccc1C(=O)O]

and
b) seven or more pharmaceutically acceptable excipients comprising a first filler, a second filler, a glidant, a disintegrant, a surfactant, a binder, and a lubricant,
   wherein CLTX-305 is present in an amount of from about 13% to about 30% by weight;
the first filler is mannitol;
the second filler is microcrystalline cellulose;
the glidant is colloidal silicon dioxide;
the disintegrant is croscarmellose sodium;
the surfactant is one or more sucrose fatty acid esters comprising sucrose palmitate;
the binder is hydroxypropyl methylcellulose;
the lubricant is magnesium stearate; and
a ratio of the compound by weight to a total weight of the seven or more pharmaceutically acceptable excipients is constant across two or more dosage strengths.

### IV. List of Abbreviations

| Abbreviation | Definition |
|---|---|
| ADH1 | Autosomal dominant hypocalcemia type 1 (ADH1) |
| AE | Adverse event |
| AESI | Adverse event of Special Interest |
| AUC | Area under the curve |
| AUC₍₀₋₂₄₎ | Area under the concentration-time curve from time 0 |
| | to 24 hours |
| AUC_{(0-inf)} | AUC extrapolated to infinity |
| AUC₍₀₋ₜ₎ | Area under the concentration-time curve from time 0 |
| | to the last measurabletime point |
| AUC₍₀₋ₜₐᵤ₎ | AUC over the dosing interval |
| BID | Twice daily |
| BMI | Body mass index |
| Ca | Calcium |
| CaSR | Calcium-sensing receptor |
| CASR | Human gene encoding the CaSR |
| cCa | Albumin-corrected blood calcium |
| Cₘₐₓ | Maximum plasma concentration |
| Cr | Creatinine |
| C_{trough} | Trough concentration |
| DL | Dose Level |
| DMC | Data Monitoring Committee |
| ECG | Electrocardiogram |
| eGFR | Estimated glomerular filtration rate |
| ET | Early termination |
| EoT | End of Treatment |
| FDA | Food and Drug Administration |
| FGF23 | Fibroblast growth factor-23 |
| FSH | Follicle stimulating hormone |
| FU | Follow-up |
| HBsAg | Hepatitis B surface antigen |
| HIV | Human immunodeficiency virus |
| hr | Hour |
| ICF | Informed consent form |
| ICH | International Council for Harmonisation |
| IgM | Immunoglobulin M |
| IND | Investigational New Drug application |
| IMP | Investigational medicinal product (CLTX-305) |
| iPTH | Intact PTH |
| Kd | Dissociation constant |
| M1 | Metabolite of CLTX-305, ether glucuronide of |
| | the parent drug |
| M3 | Metabolite of CLTX-305, acyl glucuronide of |
| | the parent drug |
| NIH | National Institutes of Health |
| PD | Pharmacodynamic |
| PK | Pharmacokinetic |
| PK/PD | Pharmacokinetic/pharmacodynamic |
| PTH | Parathyroid hormone |
| QD | Once daily |
| QT_{C} | QT interval corrected for changes in the heart rate |
| SOC | System organ class |
| t_{1/2} | Apparent terminal half-life |
| TEAE | Treatment-emergent adverse event |
| tₘₐₓ | Time to maximum plasma concentration |
| WOCBP | Women of childbearing potential |

### V. Examples

### Example 1: A Phase 2b, Open-Label Dose-Ranging Study—Protocol Summary

### 1.1 Study Summary

A Phase 2b, open-label dose-ranging study is designed to evaluate the safety, tolerability, and efficacy of CLTX-305 to maintain normalized albumin-corrected blood calcium (cCa) in subjects with hypocalcemia due to ADH1. The study consists of 2 cohorts and 3 periods, as shown in FIG. 1. An overview scheme of the Phase 2b study is illustrated in FIG. 2. A detailed scheme for Periods 1 and 2 (single and multiple ascending dose testing) of the study is shown in FIG. 3. A detailed scheme for Period 3 of the study is shown in FIG. 4.

Up to 20 male or female subjects (up to 16 to enter the treatment phase) with Autosomal Dominant Hypocalcemia Type 1 (ADH1) are enrolled in the study for evaluation.

For the study, an oral tablet containing the active ingredient CLTX-305 is provided in 10 mg, 30 mg, and 60 mg in each tablet.

Estimated time from when the study opens to enrollment until completion of data analyses is approximately 24 months. Total duration of study participation, including Periods 1, 2, and 3, can be approximately 12 months for each subject, including up to 60 days between the screening visit and study drug initiation.

### Study Objectives

Primary:

Periods 1 and 2: Evaluate the safety and tolerability of single and multiple doses of CLTX-305 in subjects with ADH1.

Period 3: Evaluate the safety, tolerability, and efficacy of CLTX-305 in subjects with ADH1 after 24 weeks of dosing.

Secondary:

Periods 1 and 2: Evaluate the effect of CLTX-305 to increase serum PTH levels after both single and multiple doses across a dose range in subjects with ADH1.

Periods 1, 2, and 3: 1) Evaluate the pharmacodynamic (PD) effects of CLTX-305 on blood calcium concentrations; 2) Evaluate the PD effects of CLTX-305 on associated measures of calcium homeostasis including 1,25-(OH)₂ Vitamin D levels and urinary calcium excretion; and 3) Evaluate the PD effects of CLTX-305 on bone turnover markers including C-teopeptide (CTx) and procollagen type 1 N-propeptide (P1NP); 4) Evaluate the pharmacokinetic (PK) profiles of both single and multiple ascending doses of CLTX-305 in subjects with ADH1.

Exploratory:

Period 3: 1) Evaluate the effect of CLTX-305 (encaleret) treatment on nephrocalcinosis/nephrolithiasis in participants with ADH1; and 2) Evaluate the effect of CLTX-305 (encaleret) treatment on bone mineral density (BMD) in participants with ADH1.

### Study Endpoint

Primary:

Periods 1, 2, and 3: Adverse events (AEs), clinical safety laboratory tests, vital signs, and electrocardiograms (ECGs)

Period 3: Albumin-corrected blood calcium concentrations (cCa) and 24-hr urinary calcium excretion after treatment with CLTX-305 for up to 24 weeks.

Secondary:

Periods 1 and 2: iPTH blood concentration profiles (24-hours) over time after single and multiple doses of CLTX-305

Periods 1, 2, and 3:
1) Pharmacodynamic endpoints measured over time up to 24 weeks (final visit):
   Blood calcium—Absolute levels and change from baseline in cCa;
   Urinary calcium clearance (fractional excretion and 24-hour total excretion);
   Serum levels of 1,25-(OH)₂ Vitamin D;
   Blood samples for magnesium, phosphate, creatinine;
   Urine samples for pH, magnesium, phosphate, sodium, potassium, creatinine, cAMP, citrate;
   Bone resorption markers collagen cross-linked C-telopeptide (CTx); and
   Bone formation markers—blood procollagen type 1 N-propeptide (P1NP);
2) PK parameters: maximum plasma concentration (Cmax), time to maximum plasma concentration (tmax), apparent terminal half-life (t½), area under the concentration-time curve (AUC) from time 0 to the last measurable time point (AUC(0-t)), AUC from time 0 to 24 hours (AUC(0-24)), AUC extrapolated to infinity (AUC(0-inf)) following single-doses; and
3) Determination of the steady state PK parameters: Cmax, trough concentration (Ctrough), and AUC over the dosing interval AUC(0-tau).

Exploratory:

Period 3: 1) Change from baseline to scheduled time points for nephrocalcinosis and/or nephrolithiasis as assessed by renal ultrasound; and 2) Change from baseline to scheduled time points in lumbar spine BMD, total hip BMD, femoral neck BMD, distal radius BMD, and total body BMD as assessed by dual-energy x-ray absorptiometry (DXA).

### 1.2 Schedule of Activities (SOA)

Schedules of Events for Periods 1, 2, and 3 are listed in Table 1, Table 2, and Table 3, respectively.

TABLE 1
Period 1 Schedule of Events

| | | | | | | | | | | | FU |
|---|---|---|---|---|---|---|---|---|---|---|---|
| | | | | | | | | | | | Post |
| | | | NIH CC inpatient Period 1 | | | | | | | | last |
| | | Interval | | QD | | | BID | | Discharge/ | FU | dose |
| Period 1 | Screening¹ | labs¹² | Admission | Dosing | | | Dosing | | EoT/ET² | Lab³ | 9-11 |
| Days | −60 to −2 | −14 to −10 | −1 | 1 | 2 | 3 | 4 | 5 | 6 | 7-8 | 30 ± 7 |
| Informed Consent | X | | | | | | | | | | |
| Admission to clinic | X | | X | | | | | | | | |
| Demographics, | X | | | | | | | | | | |
| Medical history⁴ | | | | | | | | | | | |
| Eligibility assessment | X | | | | | | | | | | |
| Safety Lab Tests⁵ | X | | X | | | | | | | | |
| Height⁶ and weight | X | | X | | | | | X | X | | |
| Vital signs⁷ | X | | X | X | X | X | X | X | X | | |
| Physical examination | X | | X | | | | | | X | | |
| Renal Ultrasound, | X | | | | | | | | | | |
| DXA Scan | | | | | | | | | | | |
| FSH level test | X | | | | | | | | | | |
| (postmenopausal | | | | | | | | | | | |
| women) | | | | | | | | | | | |
| Blood β-HCG | X | | X | | | | | X | | | |
| pregnancy test | | | | | | | | | | | |
| (WOCBP only) | | | | | | | | | | | |
| ECG, 12-lead | X | | X | | | | | X | | | |
| AEs and SAEs, | X | | X | ←----------------------------------------→ | | | | | | | |
| Prior/Concomitant | | | | | | | | | | | |
| meds⁸ | | | | | | | | | | | |
| Study drug | | | | X | X | X | X | X | | | |
| administration⁹ | | | | | | | | | | | |

| | | | | | | |
|---|---|---|---|---|---|---|
| PK/PD blood sample | | | Tables 4A-4C, 5, | | X | |
| collection | | | 6A-6C, and 7 | | | |
| 24 hr. Urine¹⁰ | X | X | | | | |
| Outpatient laboratory | | X¹¹ | | | X³ | |
| testing | | | | | | |
| Discharge¹³ | | | | X | | |
| Telephone Contact^{14, 15} | | | | | | X |

Abbreviations: Adverse Event = AE;
Albumin = Alb;
Alkaline phosphatase = ALP;
Alanine aminotransferase = ALT;
Blood urea nitrogen = BUN;
Aspartate aminotransferase = AST;
Alkaline Phosphatase = Alk Phos;
Electrocardiogram = ECG;
BID = Twice daily;
Blood Pressure = BP;
Calcium = Ca;
Calcium Sensing Receptor = CASR;
Chloride = Cl;
Concomitant Medication = Con. Meds;
Creatinine = Cr;
Dual-energy X-ray Absorptiometry = DXA;
Early Termination = (ET);
End of Treatment = EoT;
Follow-up = FU;
Follicle-Stimulating Hormone = FSH;
Glucose = Glu;
Heart Rate = HR;
Hematocrit = Hct;
Hemoglobin = Hgb;
Lactate Dehydrogenase = LDH;
Intact Parathyroid Hormone = iPTH;
Magnesium = Mg;
National Institute of Health Clinical Center = NIH CC;
Parathyroid Hormone = PTH;
Phosphate = PO₄;
Phosphorus = P;
Potassium = K;
Pharmacodynamic = PD;
Pharmacokinetic = PK;
Prothrombin Time/Prothrombin Time = PT/INR;
Red Blood Cell Count = RBC;
Thromboplastin Time = PTT;
Serious Adverse Event = SAE;
QD = Once daily;
Total Bilirubin = Tbili;
Triglycerides = TG;
White Blood Count = WBC;
Women Of Child Bearing Potential = WOCBP
¹Screening Visit at NIH Clinical Center.
²If subject discontinues from the study early (ET) or withdraws from taking the CLTX-305 (EoT), perform related assessments, resume prior medication regimen, arrange FU labs within 1-2 days, and a FU call within 30 ± 7 days after last dose of the CLTX-305.
³Outpatient laboratory testing includes blood Cr, Ca, Alb, Mg, PO₄, iPTH, and PK sample collection on Day 7 or 8 after discharge from NIH CC on Day 6.
⁴Including CaSR mutational analysis (if not documented).
⁵Safety Labs - Chemistry (Na, K, Cl, bicarbonate, Glu, BUN, Cr, Alb, total protein, Ca, Mg, PO₄, iPTH, 25-OH Vitamin D, cholesterol, TG, AST, ALT, Tbili, Alk phos, LDH, amylase, lipase, uric acid), Hematology (RBC, Hgb, Hct, RBC indices, WBC, differential), Coagulation (PT/PTT/INR), urinalysis. HIV, viral hepatitis panel to be done at screening visit only.
⁶Height measured at screening only.
⁷Vital signs include supine or sitting blood pressure (BP) by automated cuff, heart rate (HR), and respiratory rate, to be collected q8 hours during in-patient days. Orthostatic BP and HR will be assessed once at screening and once daily during Period 1.
⁸Discontinue calcitriol on Day −1 during the admission.
⁹On Days 1, 2, & 3 subjects receive CLTX-305 per the dosing algorithm. On Day 4 & 5 subjects receive BID doses based on Days 1-3.
¹⁰24-hour urine at the screening visit and 10-14 days prior to Day 1 dosing (Ca, Mg, PO₄, Cr, Na, K, Citrate, pH).
¹¹Outpatient laboratory testing on day −14-10 includes: blood Cr, calcium, albumin, magnesium, phosphate, 25-OH Vitamin D.
¹²Interval lab tests at outpatient laboratory will be done in those for whom the screening visit is >21 days prior to Period 1.
¹³After the last dose of CLTX-305, subjects resume prior outpatient conventional treatment regimen prior to discharge.
¹⁴Telephone contact: should occur on days 9 to 11 or once calcium results are available. PI/study staff to review results with subject.
¹⁵Telephone contact: 30 ± 7 days after last dose of CLTX-305 in Period 1 to review AEs/Con meds

TABLE 2
Period 2 Schedule of Events

| | | | NIH CC inpatient Period 2 | | | | | | | FU |
|---|---|---|---|---|---|---|---|---|---|---|
| | | Interval | | BID | | | | | Discharge/ | Post |
| Period 2 | Screening¹ | Labs¹¹ | Admission | Dosing | | | | | EoT/ET² | last |
| Days | −60 to −2 | −14 to −10 | −1 | 1 | 2 | 3 | 4 | 5 | 6 | dose |
| Informed Consent | X | | | | | | | | | |
| Admission to clinic | X | | X | | | | | | | |
| Demographics, | X | | | | | | | | | |
| Medical history³ | | | | | | | | | | |
| Eligibility assessment | X | | | | | | | | | |
| Safety Lab Tests⁴ | X | | X | | | | | | | |
| Height⁵ and weight | X | | X | | | | | X | X | |
| Vital signs⁶ | X | | X | X | X | X | X | X | X | |
| Physical examination | X | | X | | | | | | X | |
| Renal Ultrasound, | X | | | | | | | | | |
| DXA Scan | | | | | | | | | | |
| FSH (postmenopausal | X | | | | | | | | | |
| women) | | | | | | | | | | |
| Blood β-HCG | X | | X | | | | | X | | |
| pregnancy test | | | | | | | | | | |
| (WOCBP only) | | | | | | | | | | |
| ECG, 12-lead | X | | X | X¹⁴ | | X¹⁴ | | X¹⁴ | | |
| AEs and SAEs, | X | | X | ←----------------------------------------→ | | | | | | |
| Prior/Concomitant | | | | | | | | | | |
| meds⁷ | | | | | | | | | | |
| Study drug | | | | X | X | X | X | X | | |
| administration⁸ | | | | | | | | | | |

| | | | | | |
|---|---|---|---|---|---|
| PK/PD blood sample | | | Tables 4A-4C, 6A-6C, | | |
| collection | | | and 7 | | |
| 24 hr. Urine⁹ | X | X | | | |
| Outpatient laboratory | | X¹⁰ | | X² | |
| testing | | | | | |
| Dispense study drug | | | | X | |
| for Period 3, Discharge¹² | | | | | |
| Telephone Contact¹³ | | | | | X |

Abbreviations: Adverse Event = AE;
Albumin = Alb;
Alkaline phosphatase = ALP;
Alanine aminotransferase = ALT;
Blood urea nitrogen = BUN;
Aspartate aminotransferase = AST;
Alkaline Phosphatase = Alk Phos;
Electrocardiogram = ECG;
BID = Twice daily;
Blood Pressure = BP;
Calcium = Ca;
Calcium Sensing Receptor = CaSR;
Chloride = Cl;
Concomitant medication = Con. meds;
Creatinine = Cr;
Dual-energy X-ray Absorptiometry = DXA;
Early Termination = (ET);
End of Treatment = EoT;
Follow-up = FU;
Follicle-Stimulating Hormone = FSH;
Glucose = Glu;
Heart Rate = HR;
Hematocrit = Hct;
Hemoglobin = Hgb;
Lactate Dehydrogenase = LDH;
Intact Parathyroid Hormone = iPTH;
Magnesium = Mg;
National Institute of Health Clinical Center = NIH CC;
Parathyroid Hormone = PTH;
Phosphate = PO₄;
Phosphorus = P;
Potassium = K;
Pharmacodynamic = PD;
Pharmacokinetic = PK;
Prothrombin Time/Prothrombin Time = PT/INR;
Red Blood Cell Count = RBC;
Thromboplastin Time = PTT;
Serious Adverse Event = SAE;
QD = Once daily;
Total Bilirubin = Tbili;
Triglycerides = TG;
White Blood Count = WBC;
Women Of Child Bearing Potential = WOCBP
¹Screening Visit at NIH CC for Cohort 2 subjects. Subjects may complete the Screening Visit as outpatients or be housed overnight at NIH CC. Cohort 1 subjects do not require re-screening assessments. Subjects who complete the screening visit midweek have the option to stay overnight at NIH CC during the intervening days prior to the start of Period 2, Day −1.
²If subject discontinues from the study early (ET) or withdraws from taking CLTX-305 (EoT), perform related assessments, resume prior medication regimen, arrange FU labs within 1-2 days. Subjects instructed to obtain outpatient laboratory testing (See Footnote 10) approximately 1-2 days after discharge from NIH CC. PI/Site staff should contact the subject within 3-5 days to review test results and provide guidance regarding clinical management as they transition to prior clinical care providers. Optionally, subjects may stay in the NIH CC overnight to complete assessments.
³Including CaSR mutational analysis (if not documented).
⁴Safety Labs - Chemistry (Na, K, Cl, bicarbonate, Glu, BUN, Cr, alb, total protein, Ca, Mg, PO₄, iPTH, 25-OH Vitamin D, cholesterol, TG, AST, ALT, Tbili, Alk phos, LDH, amylase, lipase, uric acid), Hematology (RBC, Hgb, Hct, RBC indices, WBC, diff), Coagulation (PT/PTT/INR), urinalysis. HIV, viral hepatitis panel, and iron panel to be done at screening visit only unless repeat measures are clinically indicated.
⁵Height measured at screening only.
⁶Vital signs include supine or sitting blood pressure (BP) by automated cuff, heart rate (HR), and respiratory rate, to be collected q8 hours during in-patient days. Orthostatic BP and HR will be assessed once at screening and once daily during Period 2.
⁷Discontinue calcitriol on Day −1 during the admission.
⁸Initial dose (Days 1 & 2) of CLTX-305 BID based on results from Period 1. If calcium has not increased into the normal range, then the CLTX-305 dose will be increased for Days 3-5. Subjects will undergo frequent PK/PD sampling over 24 h on Day 5.
⁹24-hour urine at the screening visit 14 to 10 days prior to Day 1 dosing (Ca, Mg, PO₄, Cr, Na, K, Citrate, pH)
¹⁰For cohorts 1 and 2: outpatient laboratory testing on days −14 to −10 before admission include: blood Cr, Ca, Alb, Mg, PO₄, 25-OH Vitamin D, Hematology (RBC, Hgb, Hct, RBC indices, WBC)
¹¹Interval lab tests at outpatient laboratory will be done in those for whom the screening visit is >21 days prior to Period 2.
¹²After the last dose of CLTX-305, for subjects continuing to Period 3, dispense supply of CLTX-305 before discharge.
¹³ET and EoT - a final FU call should occur within 30 ± 7 days from the date of the last dose of CLTX-305 in Period 2.
¹⁴ECG assessments: Day 1, 3 and 5 each at 3 hrs ± 30 min post dose (AM)

TABLE 3
Period 3 Schedule of Events

| Period 3- After discharge in Period 2 (Day 6) | | | | | | | | | | | |
|---|---|---|---|---|---|---|---|---|---|---|---|
| Study Dosing Period | | | | | | | | | | | |
| | | | NIH | | NIH | | NIH CC | FU | FU | FU- | |
| | 1, 2, | | CC | | CC | | EoT/ET¹ | outpatient | outpatient | after | |
| Weeks | 3, 4 | 6 | 8 | 12 | 16 | 20 | 24 | labs² | labs² | last dose | Un-scheduled |
| Days (windows) | ±2 | ±5 | ±5 | ±5 | ±5 | ±5 | ±5 | 2 to 3 ± 2 days | 7 ± 2 days | 30 ± 7 days | visit³ |
| Telephone Contact (NIH | X | X | | X | | X | X | X | X | X | X |
| PI/Study Staff) | | | | | | | | | | | |
| AEs and SAEs, | X | X | X | X | X | X | X | | | X | X |
| Con meds | | | | | | | | | | | |
| Review | X | X | X | X | X | X | X | | | | |
| compliance⁴/Titration | | | | | | | | | | | |
| Outpatient laboratory⁵ | X | X | | X | | X | | X | X | X | |
| NIH CC visit | | | X | | X | | X | | | | |
| Renal ultrasound, | | | | | | | X | | | | |
| DXA scan | | | | | | | | | | | |
| Dispense study drug | | | X | | X | | | | | | |
| Height⁶ and weight | | | X | | X | | X | | | | |
| Vital signs⁷ | | | X | | X | | X | | | | |
| Physical examination⁸ | | | X | | X | | X | | | | |
| Safety Laboratory⁹ | | | X | | X | | X | | | | |
| Blood β-HCG | | | X | | X | | X | | | | |
| pregnancy test | | | | | | | | | | | |
| (WOCBP only) | | | | | | | | | | | |
| ECG, 12-lead | | | X | | X | | X | | | | |
| Study drug | | | X | | X | | X | | | | |
| administration¹⁰ | | | | | | | | | | | |
| PD blood collection¹¹ | | | X | | X | | X | | | | |
| PK blood collection¹² | | | | | | | X | | | | |
| Timed Interval & 24-hour | | | X | | X | X¹⁴ | X | | | X¹⁴ | |
| Urine^{13, 14} | | | | | | | | | | | |

Abbreviations: Adverse Event = AE;
Albumin = Alb;
Alkaline phosphatase = ALP;
Alanine aminotransferase = ALT;
Blood urea nitrogen = BUN;
Aspartate aminotransferase = AST;
Alkaline Phosphatase = Alk Phos;
Electrocardiogram = ECG;
BID = Twice daily;
Blood Pressure = BP;
Calcium = Ca;
Calcium Sensing Receptor = CaSR;
Chloride = Cl;
Concomitant medication = Con. meds;
Creatinine = Cr;
C-telopeptide = CTX;
Dual-energy X-ray Absorptiometry = DXA;
Early Termination = (ET);
End of Treatment = EoT;
Follow-up = FU;
Follicle-Stimulating Hormone = FSH;
Glucose = Glu;
Heart Rate = HR;
Hematocrit = Hct;
Hemoglobin = Hgb;
Lactate Dehydrogenase = LDH;
Intact Parathyroid Hormone = iPTH;
Magnesium = Mg;
National Institute of Health Clinical Center = NIH CC;
Parathyroid Hormone = PTH;
Phosphate = PO₄;
Phosphorus = P;
Potassium = K;
Pharmacodynamic = PD;
Pharmacokinetic = PK;
Procollagen type 1 N-propeptide = P1NP;
Prothrombin Time/Prothrombin Time = PT/INR;
Red Blood Cell Count = RBC;
Thromboplastin Time = PTT;
Serious Adverse Event = SAE;
QD = Once daily;
Total Bilirubin = Tbili;
Triglycerides = TG;
White Blood Count = WBC;
Women Of Child Bearing Potential = WOCBP
¹If subject discontinues from the study early (ET) or withdraws from taking CLTX-305 (EoT), before completing Period 3 per protocol, subject will be asked to return to the NIH CC as soon as possible for the ET/EoT visit assessments and return the unused CLTX-305. Subject should revert to their outpatient regimen of oral calcium and active vitamin D. Subjects will go through FU activities as stated in table and footnote 2, and then will have a safety FU call 30 day ± 7 days.
²All subjects will obtain the following outpatient laboratory assessments after last dose of CLTX-305 and after re-starting their prior regimen of oral calcium and active vitamin D: blood Cr, Ca, Alb, Mg, PO₄. Follow-up (FU) call should occur within 3-5 days to review the lab results and receive guidance on clinical management of their ADH1 as they transition to their prior clinical care providers.
³Unscheduled visits: For laboratory evaluation following dose adjustment or assessment or follow-up of an AE, in which clinically indicated procedures will be performed.
⁴Document current CLTX-305 dose and calcium dosing regimen, any modifications, and reason for modifications.
⁵Outpatient laboratory testing: Blood samples for clinical laboratory panels (Na, Cl, bicarbonate, K, BUN, Cr and including Alb, Ca, Mg, PO₄, iPTH, CTX, and P1NP).
⁶Height measured only at Week 24/final visit or ET visit.
⁷Vital signs include supine or sitting blood pressure (BP) by automated cuff, heart rate (HR), and respiratory rate.
⁸Focused Physical exams: weight measurement at all visits.
⁹Safety Labs collected at the NIH CC visits- Chemistry (Na, K, Cl, bicarbonate, Glu, BUN, Cr, Alb, total protein, Ca, Mg, PO₄, 25-OH Vitamin D, Cholesterol, TG, AST, ALT, Tbili, Alk phos, LDH, amylase, lipase, uric acid), Hematology (RBC, Hgb, Hct, RBC indices, WBC, diff), Coagulation (PT/PTT/INR).
¹⁰CLTX-305 administered and recorded during the NIH CC visit/in-house PK/PD collection days.
¹¹See Table 4A-Table 4C: Non-intensive Sampling Days for PD measures and timepoints and Table 7: Research Sample Collection.
¹²See Table 4A: Use Non-intensive Sampling Days schedule for PK timepoints.
¹³See Table 4A: see time intervals for urine collections for calculation of fractional excretion rates and 24-hour total excretion of urine Ca, Mg, PO₄, Cr, cAMP, citrate, Na, K, pH during the NIH CC visits
¹⁴Outpatient 24-hr urine Ca, Mg, PO₄, Cr, Na, K, citrate, pH at 20 weeks. Participants who withdraw from taking CLTX-305 (encaleret) (EoT) before completing Period 3 per protocol or chose not to continue into the LTE will obtain 24-hr urine at 30 ± 7 days follow-up.

### 1.3 Pharmacokinetic, Pharmacodynamic, and Urine Sampling Times

Pharmacokinetic, Pharmacodynamic, and Urine Sampling Times are detailed in Table 4A-Table 4C, Table 5, Table 6A-Table 6C, and Table 7 to show different intensive samplings for Periods 1-3 of the study.

TABLE 4A
Non-intensive BID Sampling days - Period 1: Day
4 (BID Dosing) and Period 3: PK/PD sampling days

Timepoint ± 20 min.
(relative to AM dose)

Blood Assays

| | |
|---|---|
| PK¹ samples | − 15 min, +30 min, 2, 4, |
| | 8, 11, 13 hrs |
| Intact PTH | −15 min, +30 min*, 2, 4, |
| | 8, 11, 13, 17 hrs |
| Serum bone markers: CTX, P1NP | −15 min, 13 hrs |
| 1, 25-(OH)₂ Vitamin D, cAMP | −15 min, 4, 8, 13, 17, 24 hrs |
| Ca, PO₄, Mg, Cr, albumin | −15 min, +30 min*, 2, 4, 8, |
| | 11, 13, 17, 24 hrs |
| K, CK | −15 min, 13 hrs |

Urine Assays:

| | |
|---|---|
| Timed Interval Urine Collections: | −15 min, 0-4 h, 4-8 h, |
| Ca, Mg, PO₄, Cr, cAMP, pH | 8-13 h, 13-17 h, 17-24 h |
| 24-Hour urine: | 0, 24 hrs (Start collection |
| Ca, Mg, PO₄, Cr, Na, K, Citrate, pH | after first morning |
| | void and end 24 hours later) |

¹PK in Period 3 only collected at 24 weeks; and
*+30 min timepoint only collected in Period 3 at 24 weeks

TABLE 4B
Non-intensive BID Sampling Days (if CLTX-305 dose
is ≤180 mg BID) - Period 2: Days 1, 2, 3 & 4

Timepoint ± 20 min.
(relative to AM dose)

Blood Assays

| | |
|---|---|
| PK samples | −15 min, 2, 4 hrs |
| Intact PTH | −15 min, +30 min, 2, 4, 8, |
| | 11, 13, 17 hrs |
| 1, 25-(OH)₂ Vitamin D | −15 min, 4, 8, hrs |
| Ca, PO₄, Mg, Cr, albumin | −15 min, +30 min, 2, 4, 8, |
| | 11, 13, 17 hrs |
| K, CK | −15 min, 13 hrs |

Urine Assays:

| | |
|---|---|
| Timed Interval Urine Collections: | −15 min¹, 0-4 h, 4-8 h, |
| Ca, Mg, PO₄, Cr, pH | 8-13 h, 13-17 h, 17-24 h |
| 24-Hour urine: | 0, 24 hrs (Start collection |
| Ca, Mg, PO₄, Cr, Na, K, Citrate, pH | after first morning |
| | void and end 24 hours later) |

¹-15 min urine is done on Period 2 (Day 1) only

TABLE 4C
Non-intensive BID Sampling Days (if CLTX-305
dose is >180 mg BID) - Period 2: Days 3 & 4

Timepoint ± 20 min.
(relative to AM dose)

Blood Assays

| | |
|---|---|
| PK samples | 15 min, +30 min, 2, 4, |
| | 8, 11, 13 hrs |
| Intact PTH | −15 min, +30 min, 2, 4, 8, |
| | 11, 13, 17 hrs |
| 1, 25-(OH)₂ Vitamin D | −15 min, 4, 8, |
| Ca, PO₄, Mg, Cr, albumin | −15 min, +30 min, 2, 4, 8, |
| | 11, 13, 17 hrs |
| K, CK | −15 min, 13 hrs |

Urine Assays:

| | |
|---|---|
| Timed Interval Urine Collections: | 0-4 h, 4-8 h, 8-13 h, |
| Ca, Mg, P, Cr, pH | 13-17 h, 17-24 h |
| 24-Hour urine: | 0, 24 hrs (Start collection |
| Ca, Mg, PO₄, Cr, Na, K, Citrate, pH | after first morning |
| | void and end 24 hours later) |

TABLE 5
Intensive Sampling Days for QD dosing - Period 1: Days 1, 2, 3

Timepoint ± 10 min
(relative to AM dose)

Blood Assays:

| | |
|---|---|
| PK samples | −15 min, +30 min, 1, 1.5, 2, |
| | 3, 4, 6, 8, 13 hrs |
| Intact PTH | −15 min, +30 min, 1, 1.5, 2, |
| | 3, 4, 6, 8, 13, 17 hrs |
| Serum bone markers: CTX, P1NP | −15 min, 13 hrs |
| 1, 25-(OH)₂ Vitamin D, cAMP | −15 min, 4, 8, 13, 17 hrs |
| Ca, PO₄, Mg, Cr, albumin | −15 min, +30 min, 1, 2, 3, 4, |
| | 6, 8, 13, 17 hrs |
| K, CK | −15 min, 13 hrs |

Urine Assays

| | |
|---|---|
| Timed Interval Urine Collections: | −15 min¹, 0-4 h, 4-8 h, |
| Ca, Mg, PO₄, Cr, cAMP, pH | 8-13 h, 13-17 h, 17-24 h |
| 24-Hour urine: | 0, 24 hrs (Start collection |
| Ca, Mg, PO₄, Cr, Citrate, Na, K, pH | after first morning void |
| | and end 24 hours later) |

¹−15 min urine is done on Period 1 (Day 1) only

TABLE 6A
Intensive Sampling Days for BID dosing - Period 1: Day 5

Timepoints ± 10 min
(relative to AM & PM doses)

Blood Assays

| | |
|---|---|
| PK samples | TRT AM Dose: −15 min, +30 min, |
| | 1, 1.5, 2, 3, 4, 6, 8 hrs |
| | TRT PM Dose: +30 min, 1, |
| | 1.5, 2, 3, 4, 6, 15 hrs |
| Intact PTH | TRT AM Dose: −15 min, +30 min, |
| | 1, 1.5, 2, 3, 4, 6, 8 hrs |
| | TRT PM Dose: +30 min, 1, |
| | 1.5, 2, 3, 4, 6, 8, 15 hrs |
| Serum bone markers: CTX, P1NP | TRT AM Dose: −15 min, 8 hrs |
| 1, 25-(OH)₂ Vitamin D, cAMP | TRT AM Dose: −15 min, 4, 8 hrs |
| | TRT PM Dose: 4, 8, 15 hrs |
| Ca, P, Mg, Cr, albumin | TRT AM Dose: −15 min, +30 min, |
| | 1, 1.5, 2, 3, 4, 6, 8 hrs |
| | TRT PM Dose: +30 min, 1, |
| | 1.5, 2, 3, 4, 6, 8, 15 hrs |
| K, CK | TRT AM Dose: −15 min, 8 hrs |

Urine Assays

| | |
|---|---|
| Timed Interval Urine Collections: | 0-4 h, 4-8 h, 8-13 h, |
| Ca, Mg, PO₄, Cr, cAMP, pH | 13-17 h, 17-24 h |
| 24-Hour urine: | 0-24 hrs (Start collection |
| Ca, Mg, PO₄, Cr, Citrate, Na, K, pH | after first morning void |
| | and end 24 hours later) |

Abbreviation: Time Relative To = TRT

TABLE 6B
Intensive Sampling Days for BID dosing (if CLTX-
305 dose is >180 mg BID) - Period 2: Day 5

Timepoints ± 10 min
(relative to AM & PM doses)

Blood Assays

| | |
|---|---|
| PK samples | TRT AM Dose: −15 min, +30 min, |
| | 1.5, 2, 4, 6, 8 hrs |
| | TRT PM Dose: +30 min, |
| | 1.5, 2, 4, 6, 15 hrs |
| Intact PTH | TRT AM Dose: −15 min, +30 min, |
| | 1.5, 2, 4, 6, 8 hrs |
| | TRT PM Dose: +30 min, |
| | 1.5, 2, 4, 6, 8, 15 hrs |
| Ca, PO₄, Mg, Cr, albumin | TRT AM Dose: −15 min, +30 min, |
| | 1.5, 2, 4, 6, 8 hrs |
| | TRT PM Dose: +30 min, 1.5, |
| | 2, 4, 6, 8, 15 hrs |
| K, CK | TRT AM Dose: −15 min, 8 hrs |

Urine Assays

| | |
|---|---|
| Timed Interval Urine Collections: | 0-4 h, 4-8 h, 8-13 h, |
| Ca, Mg, PO₄, Cr, pH | 13-17 h, 17-24 h |
| 24-Hour urine: | 0-24 h (Start collection |
| Ca, Mg, PO₄, Cr, Citrate, Na, K, pH | after first morning void |
| | and end 24 hours later) |

Abbreviation: Time Relative To = TRT

TABLE 6C
Intensive Sampling Days for BID dosing (if CLTX-
305 dose is ≤180 mg BID) - Period 2: Day 5

Timepoints ± 10 min
(relative to AM & PM doses)

Blood Assays

| | |
|---|---|
| PK samples | TRT AM Dose: −15 min, |
| | 1.5, 4 h |
| | TRT PM Dose: +30 min, |
| | 1.5, 4, 15 h |
| Intact PTH | TRT AM Dose: −15 min, |
| | 1.5, 4, 8 hrs |
| | TRT PM Dose: +30 min, |
| | 1.5, 4, 8, 15 hrs. |
| Serum bone markers: CTX, P1NP | TRT AM Dose: −15 min 8 hrs. |
| 1, 25-(OH)₂ Vitamin D, cAMP | TRT AM Dose: −15 min 4, 8 hrs. |
| | TRT PM Dose: 4, 8, 15 hrs. |
| Ca, PO₄, Mg, Cr, albumin | TRT AM Dose: −15 min, |
| | 1.5, 4, 8 hrs. |
| | TRT PM Dose: +30 min, |
| | 1.5, 4, 8, 15 hrs. |
| K, CK | TRT AM Dose: −15 min, 8 hrs. |

Urine Assays

| | |
|---|---|
| Timed Interval Urine Collections: | 0-4 h, 4-8 h, 8-13 h, |
| Ca, Mg, PO₄, Cr, cAMP, pH | 13-17 h, 17-24 h |
| 24-Hour urine: | 0-24 h (Start collection |
| Ca, Mg, PO₄, Cr, Citrate, Na, K, pH | after first morning void |
| | and end 24 hours later) |

Abbreviation: Time Relative To = TRT

TABLE 7
Research Sample Collection Timepoints in Periods 1 and 3 - Period
1: Day 1 and Day 4, and Period 3: Weeks 8, 16, and 24

Timepoint ± 10 min
(relative to AM dose)

Research Specimens:

| | |
|---|---|
| Including FGF23 and mid-molecule PTH | −15 min, +30 min, |
| | 2, 4, 8, 11, 13 hrs |

### Example 2: A Phase 2b, Open-Label Dose-Ranging Study—Introduction

### 2.1 Pharmacokinetics and Product Metabolism

CLTX-305 (Nonproprietary name: encaleret, molecular formula (C₂₉H₃₃ClFNO₄)₂.H₂SO₄.H₂O, molecular weight 1144.15) is formulated as 10, 30 and 60 mg round, white film coated tablets for the current clinical trial. The active agent inhibits the CaSR, via allosteric modulation, with an IC₅₀ of 86.2+6.2 nM (in cell-based assays at 2 mM extracellular calcium). In humans, two major glucuronide metabolites (M1 and M3) occur. M1 (IC₅₀>3000 nM) is inactive and M3 (IC₅₀ 67.3+6.6 nM) is active.

CLTX-305 is rapidly absorbed in humans (tₘₐₓ median 1.5 hr. (range 0.75-3 h at most single doses or at steady-state). The elimination half-life (t_{1/2}) was approximately 6.5 hours after single doses and 11-14 hours at steady state (14 days). Dose-proportional increases in plasma CLTX-305 concentrations were documented over the dose range (5, 10, 15, 30, 50, and 100 mg) in the fasted state without significant food effect. CLTX-305 showed minimal accumulation when administered once daily (QD) for two weeks in Japanese postmenopausal women. Based on non-clinical data, fecal excretion was the main route of elimination. In humans, excretion of unchanged CLTX-305 and metabolites into urine was minimal (<5% as total of parent and metabolite).

The two major glucuronide metabolites showed similar pharmacokinetics as the parent drug, with M1 median tₘₐₓ approximately 2 hours. and M3 median tₘₐₓ approximately 1.5 hours. For M1 (inactive), concentrations in plasma were approximately 2-fold higher than those of parent drug; for M3 (active) concentrations were low (10% compared to parent). Neither M1 nor M3 showed prolonged elimination.

No significant age-related or race differences (US population vs. Japanese subjects) were observed in CLTX-305 pharmacokinetics. AUC and Cₘₐₓ values may be approximately 37% and 30% higher in females but these differences were not significant when body weight was taken into account. Hepatic and renal impairment studies have not been conducted.

In a drug interaction study, co-administration with ketoconazole, a potent CYP3A4 inhibitor, resulted in approximately 1.67× increase in Cₘₐₓ and approximately 2× increase in AUC₀₋₂₄ with slightly less effect on M1 and M3.

### 2.2 Efficacy of CLTX-305 in Prior Clinical Trials

During the prior development program in osteoporosis, dose-related increases of endogenous serum parathyroid hormone (PTH) and calcium (Ca) were observed in subjects with presumed normal CaSR function (i.e. healthy volunteers and postmenopausal women with osteoporosis). In healthy postmenopausal women, repeat CLTX-305 doses of 15 or 20 mg resulted in consistent elevations in intact PTH (iPTH) over 3-4 hours post single daily dosing. The stimulatory effect on iPTH was consistent, without evidence for tachyphylaxis, after 6 and 12 months of once daily administration. CLTX-305 dosing in phase 2 studies was limited to 15 mg once daily (QD) to minimize hypercalcemia.

With chronic once-daily dosing up to 15 mg, albumin-corrected blood calcium (cCa) levels reached a plateau with up to 30% of subjects registering an elevated blood calcium above the upper limit of the laboratory reference range during long-term trials in postmenopausal women with normal systemic calcium homeostasis (normal CaSR sensitivity). Such hypercalcemia was asymptomatic, modifiable by decreasing calcium/vitamin D supplementation, and/or resolved after stopping CLTX-305 dosing. No serious adverse events (SAEs) related to either hyper- or hypocalcemia were observed in the CLTX-305 program for osteoporosis in postmenopausal women.

In summary, extensive data from the prior osteoporosis development program demonstrated that CLTX-305 is associated with dose-proportional increases in peak serum iPTH levels in subjects presumably expressing wild-type CaSR, with iPTH elevations lasting less than 8-12 hours but associated with elevations in cCa at chronic daily oral doses of 15 mg or above.

### 2.3 Risk/Benefit Assessment

### Known Potential Risks

CLTX-305 has been generally well tolerated in humans during chronic treatment up to and beyond 12 months. In a prior osteoporosis development program, CLTX-305 was administered to over 1300 healthy males and post-menopausal women; no specific safety issues or signals were seen with acute dosing up to 100 mg and chronic dosing up to 15 mg QD. No distinct patterns of adverse events (AEs), to suggest intolerance nor evidence of toxicity (on- or off-target), were seen with CLTX-305, except for its known, on-target pharmacological effect to stimulate endogenous PTH hormone release and dose-related hypercalcemia (in euparathyroid subjects) as described above.

The most common adverse events reported in early phase trials included constipation, headache, dizziness, nausea, vomiting, abdominal pain, back pain and dermatitis in addition to increased blood calcium concentrations. None of these adverse events occurred with differential frequency in the larger phase 2 trials where no specific pattern of adverse events or clear differences were evident in AEs by System, Organ, Class between CLTX-305 and placebo or comparator arms (other than differences in calcium levels).

A thorough QT study in healthy subjects showed no clinically meaningful QTc prolongation following a single oral dose of up to 100 mg CLTX-305 at any of the examined timepoints.

In preclinical safety and toxicology studies the dose-limiting effects of CLTX-305 were related to mechanism-based elevations in serum calcium concentrations and effects on calcium homeostasis. In contrast, no off-target or other tissue or organ-specific toxicities were identified.

It is not known if CLTX-305 could have an effect on pregnancy or breastfeeding in humans. CLTX-305 may have potential teratogenic effects in humans based on the results of a reproductive toxicity study in rabbits where the incidence of skeletal anomalies (fusion of the sternebra) was increased in the fetuses at 30 mg/kg and above. Currently, Women of child-Bearing Potential (WOCBP) will be consented to receive pregnancy testing at screening and at intervals throughout the trial and required to utilize contraception and agree to avoid pregnancy during the experimental treatment.

There are currently no approved alternative therapies for people with ADH1 to maintain blood calcium levels in the normal or low-normal range other than oral calcium and active vitamin D supplementation. The activating mutation of the CASR leads to an insufficient iPTH response to low blood levels of calcium such that these patients are hypocalcemic without therapy. The goal of therapy is to raise blood calcium concentrations sufficiently to improve/abrogate symptoms and improve function and well-being. Current therapy with oral supplements presents challenges due to the frequency and pill-burden of using calcium and active vitamin D as well as the iatrogenic complication related to hypercalciuria which results from increasing the overall calcium load. These complications of current therapy often include nephrocalcinosis, progressive renal dysfunction and can result in renal failure. Treatment with CLTX-305 is being explored as a potential alternative or adjunctive treatment to maintain blood calcium levels while minimizing hypercalciuria.

In the dose-escalating and dose-finding components of Periods 1, 2 and 3, participants may experience fluctuations in their blood calcium levels including the potential for hypocalcemia and/or hypercalcemia. The long-term risk of such fluctuations with CLTX-305 are not expected to be larger than the risk of current therapy however such fluctuations may be more frequent during the initial dose-escalating/dose finding portions of the clinical study. The clinical study is designed to minimize the risk of untoward fluctuations in blood calcium and related serum chemistries: During the dose-finding Periods 1 and 2, subjects will be supervised with frequent monitoring of blood calcium levels multiple times per day and may receive treatment for hypocalcemia or hypercalcemia as required while effective doses of CLTX-305 are being identified. Each subject is assessed for evidence of an individualized effective dose of CLTX-305 during the in-house periods before entering outpatient Period 3. During Period 3 subjects continue a combination of their oral calcium supplements and individualized starting dose of CLTX-305 and will undergo at least weekly re-assessment and dose-titration based on outpatient laboratory monitoring results and telephone contact visits with the investigative site staff. The outpatient approach to dose and regimen modification is similar to the current practice for optimizing conventional treatment with oral calcium and active vitamin D.

Additional risks and potential discomforts related to the frequent blood and urine sampling procedures in the supervised setting of the in-house stays during Periods 1 and Periods 2 are addressed in the Informed Consent Document and include evaluations of medical history, physical examination, renal ultrasound and EKG. During test days multiple blood and urine collections will occur introducing known common risks (discomfort, bruising) for blood draws and/or intra-venous catheter placement introducing smaller risks of complications including fainting (usually a vasovagal response to sight or insertion of needles) or inflammation of the insertion site (including potentially pain and swelling) and the potential for infection. These will be treated if they occur.

Based on the totality of the evidence acquired to date, no other organ-specific or adverse safety signals of potential clinical concern have been identified. Clinical trials in the new target population of patients with ADH1 will closely evaluate dose-related effects on calcium homeostasis and otherwise employ routine safety monitoring (For details on CLTX-305 safety data, see D3 Section 5.3).

### Known Potential Benefits

There are no definitive currently known benefits of CLTX-305 in the target population of patients with hypocalcemia due to ADH1. In vitro and in vivo studies, including preclinical data in mouse models suggest that calcilytic agents including CLTX-305 may shift the sensitivity of the ADH1-mutated CaSR back towards “normal” resulting in normalization of blood calcium levels. In a pilot study in humans with ADH1, an investigational calcilytic was shown to dose-dependently stimulate PTH acutely associated with transient suppression of urinary calcium excretion although the doses and times tested may have been insufficient to demonstrate definitive changes in serum calcium levels (Roberts et al., Journal of bone and mineral research: the official journal of the American Society for Bone and Mineral Research, 2019).

### Assessment of Potential Risks and Benefits

Taken together, both preclinical and available clinical data support further clinical development of CLTX-305 as a potential therapy targeted to the molecular defects in ADH1. Current standard of care, which includes oral calcium and active vitamin D supplementation, is complicated by the increased sensitivity of the CaSR to exogenous calcium which leads to an exaggerated urinary calcium excretion in response to small increases in blood calcium. This augmented urinary calcium excretion occurs both because iPTH levels are sub-optimal due to the altered CaSR function in the parathyroid glands, and due to the over-stimulation of the CaSR in the distal renal tubules, both of which contribute to enhanced urinary calcium loss and are associated with chronic complications of renal function. CLTX-305, as a calcilytic agent, may dampen the CaSR to facilitate achievement and maintenance of the therapeutic targets of normalized blood calcium concentrations while also avoiding or minimizing hypercalciuria.

One challenge to clinical development of CLTX-305 is that the doses of CLTX-305 that will be effective in patients with ADH1 may be higher than the doses previously identified as raising blood calcium in extensive clinical experience in euparathyroid subjects (postmenopausal women in the prior osteoporosis program). Effective doses of CLTX-305 for patients with ADH1 cannot be determined without execution of a specifically designed clinical trial. The current proposed clinical trial is designed to utilize conventional early drug development approaches, including single and multiple-ascending doses, while leveraging prior safety exposure data, to address the important questions of dose-response, acute safety and tolerability, and definitive proof-of-concept regarding the extent to which CLTX-305 can act to normalize blood calcium in subjects with ADH1.

The study is designed to minimize known potential risks (as described above) while maximizing the range of doses studied to increase the probability of establishing a definitive proof-of-concept of the potential utility of CLTX-305 as a treatment for hypocalcemia in patients with ADH1.

### Example 3: A Phase 2b, Open-Label Dose-Ranging Study—Objectives and Endpoints

TABLE 8
Objectives and Endpoints

| | |
|---|---|
| Objectives | Endpoints |

Primary

| | |
|---|---|
| Periods 1 and 2 | Periods 1, 2, and 3: |
| Evaluate the safety and tolerability of single and multiple | Adverse events (AEs), clinical safety laboratory tests, vital |
| doses of CLTX-305 in subjects with ADH1 | signs, and electrocardiograms (ECGs) |
| Period 3: | Period 3: |
| Evaluate the safety, tolerability and efficacy of CLTX-305 | Albumin-corrected blood calcium concentrations (cCa) |
| in subjects with ADH1 for 24 weeks | after treatment with CLTX-305, 24-hr urinary calcium |
| | excretion for 24 weeks. |

Secondary

| | |
|---|---|
| Periods 1 and 2: | Periods 1 and 2: |
| Evaluate the effect of CLTX-305 to increase serum iPTH | iPTH blood concentrations profiles (24-hours) over time |
| levels after both single and multiple doses across a dose | after single and multiple doses of CLTX-305 |
| range in subjects with ADH1 | |
| Periods 1, 2, and 3: | Periods 1, 2, and 3: |
| Evaluate the pharmacodynamic (PD) effects of CLTX-305 | Pharmacodynamic endpoints measured over time up to |
| on blood calcium concentrations; | 24 weeks (final visit): |
| Evaluate the PD effects of CLTX-305 on associated | Blood calcium - Absolute levels and change from baseline |
| measures of calcium homeostasis including 1,25-(OH)₂ | in cCa |
| Vitamin D levels and urinary calcium excretion; and | Urinary calcium clearance (fractional excretion and |
| Evaluate the PD effects of CLTX-305 (encaleret) on bone | 24-hour total excretion) |
| turnover markers including C-telopeptide (CTx) and | Renal function (eGFR) |
| procollagen type 1 N-propeptide (P1NP) | Serum levels of 1,25-(OH)₂ Vitamin D |
| | Blood samples for magnesium, phosphate, creatinine |
| | Urine samples for pH, magnesium, phosphate, sodium, |
| | potassium, creatinine, cAMP, citrate |
| | Bone resorption markers collagen cross-linked C- |
| | telopeptide (CTx) |
| | Bone formation markers - blood procollagen type 1 |
| | N-propeptide (P1NP) |
| Evaluate the PK of both single and multiple ascending | PK parameters: maximum plasma concentration (Cₘₐₓ), |
| doses of CLTX-305 in subjects with ADH1. | time to maximum plasma concentration (tₘₐₓ), apparent |
| | terminal half-life (t_{1/2}), area under the concentration-time |
| | curve (AUC) from time 0 to the last measurable time point |
| | (AUC₍₀₋ₜ₎), AUC from time 0 to 24 hours (AUC₍₀₋₂₄₎), AUC |
| | extrapolated to infinity (AUC_{(0-inf)}) following single-doses. |
| | Determination of the steady state PK parameters: C_{max,} |
| | trough concentration (C_{trough}), and AUC over the dosing |
| | interval AUC₍₀₋ₜₐᵤ₎ |

### Example 4: A Phase 2b, Open-Label Dose-Ranging Study—Study Design

### 4.1 Overall Design

This is a single-site, open-label, dose-ranging study to evaluate the safety, tolerability and efficacy of CLTX-305 to maintain normal albumin-corrected calcium (cCa) in subjects with hypocalcemia due to ADH1. The study consists of 2 cohorts and 3 periods, as outlined in FIG. 2.

Two main cohorts are planned:
Cohort 1: up to 8 subjects (minimum of 5) are initially enrolled into Period 1 (inpatient), after which they may return to participate in Period 2 (inpatient, after at least an 8-week interval). Subjects who complete Period 2 are eligible to enter Period 3 (outpatient) for up to 24 weeks of dosing with CLTX-305.
Cohort 2: up to 8 additional subjects (minimum of 5) enroll directly into Period 2 (inpatient). The initiation of Cohort 2 will be based on evaluation of accumulated data from Cohort 1. Cohort 2 subjects who complete Period 2 are eligible to enter the outpatient Period 3 (outpatient) to receive up to 24 weeks of CLTX-305.

Subjects from both cohorts who complete Period 2 will be eligible to enter Period 3. Outpatient dosing with CLTX-305 will be determined from review of their individual dose response data from Periods 1 and/or 2 based on identifying well-tolerated BID doses with preliminary evidence for efficacy. During the outpatient period (Period 3), subjects will undergo additional individualized dose titration as necessary in response to safety data and the results of key efficacy measures.

Periods 1 and 2 of the study are detailed in FIG. 3. Period 3 of the study is detailed in FIG. 4.

Period 1: an inpatient stay that consists of 5 dosing days during which subjects will undergo a once-daily (QD) dose escalation for 3 days followed by 2 days of twice daily (BID) dosing at an individualized test dose of CLTX-305.

Period 2: an inpatient stay that consists of 5 dosing days during which subjects will receive BID doses of CLTX-305 based on individual responses from Period 1 (for subjects who complete Period 1) or review of aggregate data from Period 1 (for subjects in Cohort 2 entering Period 2 without prior exposure to CLTX-305). The initial dose, dose level 1 (DL1) will be administered for 2 days (48 hours) with cCa monitoring. Participants will undergo encaleret dose up- or down-titration depending on cCa levels, with a potential increase in dose if cCa remains below the lower limit of normal and potential decrease in dose if cCa is greater than or equal to the upper limit of normal. A final test day (Day 5) will include frequent blood and urine sampling to collect 24-hour PK/PD profiles after at least 3 consecutive days of dosing (at either DL1 or DL2).

Period 3: outpatient dosing with CLTX-305 for up to 24 weeks. Subjects completing Period 2 will continue to self-administer CLTX-305 at an initial BID dose based on their tolerance and response to BID dosing during Period 2. Initial titration will be based on each subject's need for symptom control and ongoing monitoring of efficacy endpoints (primarily cCa and urine calcium excretion) with a goal to optimize cCa in the normal range while minimizing hypercalciuria. Subjects will not take calcitriol but may take additional calcium supplementation as needed if a minimum daily dietary intake of about 1000 mg cannot be achieved and the subject has persistent hypocalcemia. Titration of oral calcium supplements will be directed by the investigative site during the outpatient Period and may be up or down titrated based on achieving the therapeutic goal of a normal or low-normal cCa without hypercalciuria. During Period 3, a titration phase will extend up to 12 weeks after which each individualized dose regimen will continue to be administered with a goal to maintain a stable dose, a stable normal to low-normal cCa while avoiding hypercalciuria. The need for additional dose adjustments may depend on factors potentially related to the time course of changes in parathyroid gland function, intestinal calcium absorption, bone resorption, and kidney function, all of which will be monitored.

### 4.2 Scientific Rationale for Study Design

The current clinical trial is designed to test CLTX-305 across a range of doses administered both once and twice daily to determine the ability of a calcilytic to raise blood calcium in patients with ADH1.

The current study is designed to confirm the utility of the calcilytic agent, CLTX-305, to treat hypocalcemia due to ADH1 through allosteric antagonism of the mutated CaSR in these patients. The study is designed to identify minimally effective doses, steady state pharmacokinetic/pharmacodynamic (PK/PD) relationships, and/or maximally tolerated doses in the target population of people with confirmed ADH1. Such data will be established during in-house periods 1 & 2 resulting in individualized dosing that will inform starting doses for outpatient exposure during Period 3. Initial outpatient dosing may be further titrated, along with oral calcium supplements, with the goal of optimizing blood calcium without the need for calcitriol while minimizing urinary Ca excretion.

Due to the orphan nature of ADH1, and the rarity of available subjects for participation in this clinical trial, the Sponsor believes it is reasonable to anticipate extending the study into an outpatient Period 3 based on the expectation that Periods 1 and 2 will identify both individualized and rational average effective doses across both Cohorts 1 and 2.

### 4.3 Justification for Dose

The current study proposes to initiate first-in-patient dosing, in the new target population of subjects with ADH1, at 30 mg of CLTX-305. This dose has been shown previously to be safe and well tolerated in non-ADH1 subjects (i.e., people not harboring pathogenic, gain-of-function variants that drive ADH1) and was unequivocally associated with increased plasma iPTH and elevation of cCa. It is expected that this may be an ineffective or minimally effective dose in ADH1 and therefore represents a logical starting dose upon which to base dose-escalation and dose-finding. For details of the PK/PD relationship of CLTX-305 in euparathyroid subjects with presumed normal CaSR function.

Using both single ascending and multiple-ascending dose paradigms, the proposed study will explore the PK-PD relationship of CLTX-305 in subjects with ADH1. Because ADH1 is an orphan disease with few identified potential subjects, and because of the well characterized safety data available from the prior development program, we propose to conduct individualized dose escalation in a cohort of approximately 16 subjects. It is not possible to predict what dose of CLTX-305, on average, will result in elevated calcium in ADH1 but it is anticipated that dosing may need to proceed from 30 mg through at least a log increase. The supervised in-house testing during Periods 1 and 2 will allow dosing to proceed based on safety and tolerability with close monitoring for evidence of efficacy based on changes in iPTH and cCa for each subject. Doses that elevate cCa, for example >1 mg/dL, within 1-5 days, would establish a definitive proof-of-concept for CLTX-305 as a potential treatment for ADH1. Doses that are associated with hypercalcemia would establish an upper bound or maximally tolerated dose.

Given that Periods 1 and 2 are designed to identify individually effective doses of CLTX-305, as well as potential upper bounds, these results should facilitate the choice of outpatient starting doses, likely at lower doses, as well as inform the initial approach to outpatient dose adjustment and optimization during Period 3.

Period 3 is designed to identify individualized chronic doses. Due to the different timeframes for PTH action (relatively immediately limiting calcium loss in the urine, followed by increasing endogenous 1,25-(OH)₂ Vitamin D effects on GI absorption, and eventually mobilizing bone calcium), chronic outpatient doses may be lower than doses shown to be acutely effective. The dose-response curves for both iPTH stimulation and cCa elevation from Periods 1 and 2 may also inform updated PK-PD models which are currently based on non-ADH1 subjects (Cabal et. al., Journal of bone and mineral research, 2013, 28 (8), pp. 1830-1836). Taken together, individualized subject data and the ongoing accrual of data on all subjects should facilitate rational outpatient dosing and titration.

### Example 5: A Phase 2b, Open-Label Dose-Ranging Study—Study Population

### A. Inclusion Criteria

Subjects meet the following criteria for inclusion during screening:
1) Be able to understand and sign a written informed consent or assent form, which must be obtained prior to initiation of study procedures;
2) Age≥16 years;
3) Postmenopausal women are allowed to participate in this study:
   a) Women are considered postmenopausal and not of child bearing potential if they have had 12 months of natural (spontaneous) amenorrhea with an appropriate clinical profile (e.g. age appropriate, history of vasomotor symptoms) or have had surgical bilateral oophorectomy (with or without hysterectomy) or tubal ligation at least 6 weeks prior to start of the study. In the case of oophorectomy alone, only when the reproductive status of the woman has been confirmed by follow up hormone level assessment, shall she be considered not of childbearing potential.
4) Body mass index (BMI)≥18.5 to <39 kg/m²;
5) Have an activating mutation of the CASR gene;
6) Subjects being treated with thiazide diuretics may be enrolled if they are willing and able to discontinue thiazides for at least 5 half-lives prior to initiation of CLTX-305 and during the study treatment period. When the thiazide is being used as an antihypertensive, alternative therapy will be offered; and
7) Subjects being treated with strong CYP3A4 inhibitors (including clarithromycin, telithromycin, nefazodone, itraconazole, ketoconazole, atazanavir, darunavir, indinavir, lopinavir, nelfinavir, ritonavir, saquinavir, tipranavir) should ideally, if clinically appropriate, discontinue these medications during the screening period for at least 5 half-lives prior to initiation of CLTX-305. Subjects who must remain on strong CYP3A4 inhibitors may still enroll if they are able to remain on their medications at stable doses throughout the trial.
8) Subjects being treated with magnesium or potassium citrate supplements should discontinue such treatment starting on Day −1 during Period 1 and Period 2 and may be asked to discontinue treatment during Period 3.

### B. Exclusion Criteria

Subjects who meet any of the following criteria during screening are not eligible to participate in the study:
1). History of treatment with PTH 1-84 or 1-34 within the previous 3 months;
2). History of hypocalcemic seizure within the past 3 months;
3). Blood 25-OH vitamin D level<25 ng/mL;
   a) If subject has a blood 25-OH vitamin D level<25 ng/mL at the screening visit, they will be prescribed cholecalciferol or ergocalciferol supplementation. Once the 25-OH vitamin D level is >25 ng/mL, the subject will be eligible to continue to the treatment phase of the study.
4). Abnormal laboratory values which in the opinion of the investigator, would make the subject not suitable for participation in the study;
5). Estimated glomerular filtration rate (eGFR)<25 mL/minute/1.73 m² using CKD-EPI (for subjects<18 years old the Schwartz equation will be calculated);
6). 12-lead resting electrocardiogram (ECG) with clinically significant abnormalities;
7). Subjects with positive hepatitis B surface antigen (HbsAg), Hepatitis C antibody, Hepatitis A immunoglobulin M (IgM), or human immunodeficiency virus (HIV) viral serology test results at the Screening Visit;
8). Subjects with hemoglobin (Hgb)<13 g/dL for men and <12 g/dL for women;
9). Pregnant or nursing (lactating) women, where pregnancy is defined as the state of a female after conception and until the termination of gestation, confirmed by a positive serum hCG laboratory test;
10). Women of child-bearing potential, defined as all women physiologically capable of becoming pregnant, unless they are using highly effective methods of contraception during dosing and for 3 months following the discontinuation of study treatment. Highly effective contraception methods include:
   Total abstinence (when this is in line with the preferred and usual lifestyle of the subject). Periodic abstinence (e.g., calendar, ovulation, symptothermal, post-ovulation methods) and withdrawal are not acceptable methods of contraception.
   Female sterilization (have had surgical bilateral oophorectomy with or without hysterectomy) or tubal ligation at least six weeks before taking study treatment. In case of oophorectomy alone, only when the reproductive status of the woman has been confirmed by follow up hormone level assessment.
   Male sterilization (at least 6 months prior to screening). For female subjects on the study the vasectomized male partner should be the sole partner for that subject.
   Combination of the following (a+b or a+c, or b+c):
   a) Use of oral, injected or implanted hormonal methods of contraception or other forms of hormonal contraception that have comparable efficacy (failure rate<1%), for example hormone vaginal ring or transdermal hormone contraception.
   b) Placement of an intrauterine device (IUD) or intrauterine system (IUS).
   c) Barrier methods of contraception: Condom or Occlusive cap (diaphragm or cervical/vault caps) with spermicidal foam/gel/film/cream/vaginal suppository.
11). Sexually active males unless they use a condom during intercourse while taking the CLTX-305 (study drug) and for 3 months after the last dose of the study drug and should not father a child during active participation in the study starting with the first CLTX-305 dose in Period 1, Period 2 (for Cohort 2) until the end of Period 3. A condom is required to be used also by vasectomized men in order to prevent delivery of the study drug via seminal fluid.
12). Hypersensitivity to any active substance or excipient of CLTX-305;
13). History of drug or alcohol dependency within 12 months preceding the Screening Visit;
14). History of thyroid or parathyroid surgery;
15). Current participation in other investigational drug studies; and
16). Unwillingness to refrain from blood donation within 12 weeks prior to screening visit from the start of the study enrollment through one year after the last dose of the study drug.

### C. Inclusion of Vulnerable Participants

Subjects between 16 and 18 years of age may be enrolled in this study. Older adolescents have the capacity to provide adequate assent in an intensive Phase 2a study for which benefit in this disorder remains uncertain. Younger children are excluded because of the intensity of the procedures with uncertain clinical benefit.

### Example 6: A Phase 2b, Open-Label Dose-Ranging Study—Study Intervention

### 6.1 Study Interventions(s) Administration

During Period 1, subjects from Cohort 1 will receive CLTX-305 QD and BID per protocol with water as described in Example 7.

During Period 2, subjects from both Cohort 1 and Cohort 2 will receive CLTX-305 BID with water as described in Example 7.

During Period 3, subjects from both Cohort 1 and Cohort 2 who enter Period 3 will be discharged home to continue taking CLTX-305 BID with water as described in Example 7.

### 6.1.1 Study Intervention Description

The investigational medicinal product (IMP), CLTX-305, will be provided as white film-coated tablets containing the active ingredient encaleret provided in 10, 30 and 60 mg dosage strength.

The CLTX-305 will be provided as 10 mg, 30 mg, and 60 mg tablets in bottles.

### 6.1.1 Dosing and Administration

Dosing of CLTX-305 in subjects with ADH1 is informed by prior experience of both acute and chronic dosing of CLTX-305 in the osteoporosis program that included exposures up to 1 year primarily in postmenopausal women with presumed normal CaSR function. The proposed starting dose is 30 mg in ADH1 because this is a dose proven to result in acute elevations in cCa in euparathyroid subjects without an ADH1 mutations. Based on peer-reviewed data from a pilot human study of a related calcilytic agent, NPSP795 (Roberts et. al., Journal of bone and mineral research: the official journal of the American Society for Bone and Mineral Research, 2019), which also studied a top dose known to cause a rise in calcium in euparathyroid subjects but not in ADH1 subjects, we anticipate that the starting dose of 30 mg may be ineffective or minimally effective but represents a rational initial dose that has been demonstrated as safe and well tolerated previously.

Because a comprehensive safety database exists from the prior osteoporosis development program, including 12 months or more duration of safety exposure, the current first-in-ADH1 study will include repeat dosing, dose-escalation and dose individualization over time in individual ADH1 participants. Similar to cross-over designs, conducting dose-escalation within subjects should reduce variability and increase the precision of dose/response modeling compared to an approach that might use separate parallel or sequential cohorts. Up to 16 subjects studied should be feasible and allow characterization of individual and group mean dose-exposure-response profiles as well as proof-of-concept on elevation of cCa. Our approach is preferable since the ultra-orphan nature of the disease makes conventional parallel group dose finding not feasible.

CLTX-305 will be tested both as a single daily dose and as a twice daily dose. In the prior osteoporosis program CLTX-305 was administered once daily and this was seen to stimulate a transient increase (spike) in iPTH levels secretion within 2 hours and a second lower plateau level of iPTH (still elevated compared to baseline) for up to 12 hours at doses of 30 mg and above. Despite both PK (drug exposure) and PD (increased iPTH levels) lasting less than 24 hours, some evidence for increased trough cCa (measured 24 hours after last dose) suggests that once daily dosing and once-daily dose escalation should be tested in ADH1 subjects.

CLTX-305 (encaleret) may be administered within 30 minutes prior to meals. Administration with meals was shown in prior Phase 1 trials to be associated with adequate systemic absorption, prompt increases in plasma levels of iPTH but with a slightly blunted PTH Cₘₐₓ compared to administration while fasted (See Investigator's Brochure for details and results of Phase I trials). Recommendation for administration within 30 minutes prior to meals was chosen as the paradigm for ADH1 dosing because, unlike in the osteoporosis program, the goal in ADH1 patients is not to maximize PTH Cₘₐₓ but to facilitate increased iPTH levels above baseline throughout the day, adequate to raise blood cCa levels into the normal range. It is anticipated, however, that twice daily dosing may result in lower iPTH levels Cₘₐₓ and more stable iPTH level over the course of 24 hours versus single daily dosing. The protocol is designed to test this hypothesis by conducting both once-daily and twice daily dose escalation in Period 1 and twice-daily dose escalations in Period 2.

An overview and general description of the study strategy for intervention (CLTX-305 administration) is presented below. Additional detail on doses, regimens and titration algorithm/guidance for each Period can be found in Example 7 as well as in FIG. 5 and FIG. 6.

### Period 1 (Single Ascending Dose Escalation and BID PK/PD Profile)—Cohort 1

In Period 1, eligible Cohort 1 subjects are admitted to the NIH CC after completing screening tests. On days 1-3 of admission, Cohort 1 subjects will undergo single daily dosing and protocol-specified dose-escalation of CLTX-305 from the initial dose of 30 mg on Day 1, increasing to a maximum dose of 180 mg on Day 3. Based on the individualized responses to QD dose escalation an individualized dose of CLTX-305 will be chosen for BID administration on Days 4 and 5.

Days 1, 2 and 3 (QD Dose Escalations): Up to 8 subjects (minimum of 5) will undergo a single-dose, dose-escalation over the first 3 days of admission in Period 1. Morning doses will be administered on Days 1, 2 and 3, respectively, accompanied by frequent blood and urine sampling for 24-hours for PK/PD measures. CLTX-305 will be administered in the morning within 30 minutes prior to breakfast. If dose escalation is limited due to tolerability or hypercalcemia, then the investigator, in consultation with sponsor, may choose to interrupt the pre-specified dose escalation. In this case, the investigator, in consultation with sponsor, may choose to repeat the last well tolerated dose on Days 2 and 3, or titrate down as appropriate.

Dose-escalation will take place sequentially on Days 1, 2 and 3 without interim wash-out because prior extensive PK-PD data supports minimal drug accumulation over 24 hours when dosed once daily (for details see the Investigator's Brochure). Similarly, it is expected that iPTH levels will not remain elevated for 24 hours after dosing when CLTX-305 is administered once-daily. However, any carryover effects that may occur should not confound the objective of demonstrating an acutely efficacious dose of CLTX-305. For any given subject, cCa values>10.5 mg/dL, during a dosing interval, will indicate that a top dose of CLTX-305 has been reached for that individual and further dose escalation will not be necessary. In this scenario, the investigator, in consultation with sponsor, may choose to repeat the last well-tolerated dose on the remaining QD testing days (e.g. Days 2 and 3), as appropriate. Detailed examples of dose and titration modifications based on the cCa response are shown in FIG. 5.

Days 4 and 5 (BID Dose Escalation): Based on each subject's individual PD responses to QD dose-escalation, an individualized dose of CLTX-305 will be chosen for BID dosing on Days 4 and 5. CLTX-305 will be administered once in the morning and once in the evening, within 30 minutes prior to breakfast and dinner. The dose selected should be based on each subject's response in cCa and/or PTH levels during the prior QD dosing (Days 1-3). A guidance algorithm for BID dosing, based on the individual response to QD dosing in Period 1, is presented in FIG. 5

### Period 2 (BID Dosing)—Cohorts 1 and 2

In Period 2, eligible Cohort 1 subjects, who have completed Period 1, may return after at least 8 weeks for admission to NIH for Period 2. After all Cohort 1 subjects have finished Period 1 and the data analyzed, the Sponsor will open enrolment for Cohort 2 subjects. Eligible Cohort 2 subjects may enroll directly into Period 2, after completing the screening and consent process per protocol. In Period 2 all subjects will initiate CLTX-305 BID dosing given within 30 minutes prior to breakfast and dinner on Days 1 and 2. cCa concentrations will be monitored frequently throughout Period 2, and CLTX-305 doses will be up- or down-titrated as needed to target normal cCa concentrations. Assessment of the 24-hour PK/PD profile of CLTX-305 with BID dosing will be conducted on Day 5. A guidance algorithm for initiating and titrating BID dosing during Period 2 is presented in FIG. 6.

### Period 3 (Cohort 1 and Cohort 2)—Outpatient Dosing

In Period 3, the initial outpatient dose of CLTX-305 will be individualized based on the results from inpatient Periods 1 and/or 2 as described in greater detail in Example 7. Period 3 will include a titration phase of approximately 12 weeks and a maintenance phase of approximately 12 weeks for a total outpatient exposure to CLTX-305 of 24 weeks. Titration will be conducted by the NIH investigators at scheduled or unscheduled telephone contact visits based on review of outpatient chemistry results for cCa, Mg, phosphorous at NIH CC visits (approximately every 8 weeks) based on assessment of both blood and urine calcium results as described, with the goal of optimizing the CLTX-305 dose without calcitriol, targeting normal cCa and phosphorus concentrations, avoiding symptoms of hypo- or hypercalcemia, and minimizing the extent of hypercalciuria. Oral calcium supplementation may be used as needed on top of a minimum daily dietary intake of at least 1000 mg.

### Dose Limiting Toxicity

The goal of the current study in subjects with ADH1, is to elevate cCa concentrations, including identifying doses capable of raising cCa beyond the normal range. It is important to establish if an allosteric modulator of the CaSR is capable of causing hypercalcemia at all, in patients with activating mutations of the CaSR. The study is designed to monitor for cCa elevations in a closely supervised and expert setting familiar with acute treatment and intervention, if necessary. In the prior osteoporosis program, hypercalcemia, identified by laboratory testing, was confirmed as a dose-limiting effect in presumed euthyroid subjects without CASR mutations, however, of note, laboratory hypercalcemia was not clearly associated with hypercalcemic symptoms; the events were detected in routine laboratory monitoring and were managed by withholding or lowering the dose of CLTX-305.

Another inherent study objective is assessment of safety and tolerability across different doses and regimens including characterizing dose-limiting adverse events, determining maximum doses associated with acceptable safety risk, as well as determining doses that can effectively raise or maintain stable normal cCa concentrations.

Based on experience accumulated in human clinical testing, from the prior osteoporosis program, no specific safety signal or adverse event profile was identified with acute or chronic dosing with CLTX-305. Dose-limiting hypercalcemia was the only signal that required chronic doses to be limited to 15 mg QD in an otherwise healthy population of postmenopausal women treated for up to 12 months.

Since there is no specifically expected adverse event profile or anticipated dose-limiting toxicity in humans, safety monitoring for CLTX-305 will be based on established approaches and principles. Protocol-specified safety measures based on adverse event monitoring, safety laboratories, vital signs, physical exam, and EKGs will be assessed during the in-house Periods 1 and 2. For any individual subject, the experience of an adverse event will be evaluated and recorded according to the guidance given in Example 7, and appropriate action with respect to study drug administration will be made according to the judgment and assessment of the investigator.

It is expected that higher doses of CLTX-305 will be necessary to see comparable changes in cCa in subjects with ADH1 compared to previously tested subjects with normal CaSR function and therefore the current protocol stipulates potential dose escalation higher than 100 mg including doses possibly >400 mg acutely in order to identify effective doses in the new target population of subjects with abnormal CaSR function where cCa elevations would represent efficacy and not a safety side-effect.

The proposed starting dose for subjects with ADH1 is 30 mg, which was well tolerated, in healthy normal volunteers, without a specific safety signal, but was also rapidly effective in raising cCa. It is expected that this dose will not be effective or may be minimally effective in raising cCa in ADH1 subjects. Therefore, based on known safety and efficacy on cCa, CLTX-305 30 mg represents a rational initial dose for this first-in-ADH1 trial. Moreover, CLTX-305 was acutely safe and well tolerated across a range from 5-100 mg in non-ADH1 subjects (approximately 10-fold range). Original Phase 1, single-escalating dose trials in healthy humans with presumed normal CaSR function, were designed to escalate up to 400 mg CLTX-305; however, a top dose of 100 mg was identified based on rising cCa concentrations. The top dose in people with presumed normal CaSR function was chosen as 100 mg, to avoid excessive cCa elevations, and was used safely as the top dose in a Phase I thorough QTc study.

Of note, although people with ADH1 may show a right-shifted dose-response compared to healthy non-ADH1 people, there is no biological reason to expect the dose-response trajectory to be significantly steeper in subjects with ADH1. Therefore, it is important to plan for appropriate dose-escalation in this first-in-ADH1 trial, to focus on proof-of-calcium-raising effect. The planned dose range for QD exploration in ADH1 subjects includes a range from 30 to 180 mg (approximately 6-fold range). If higher doses are required to confirm efficacy on cCa, dose escalation will continue but switch to BID regimens as specified in greater detail in the algorithm/guidance in Example 7.

In the prior osteoporosis program, hypercalcemia adverse events were not symptomatic and were identified based on scheduled periodic laboratory assessments. Dose-limiting thresholds for post-CLTX-305 cCa levels are specified as part of the dosing and titration algorithms in Example 7. In Summary, subjects who achieve a post-dose cCa>10-10.5 mg/dL) in Period 1) and cCa>10 mg/dL (in Period 2) during the dose escalation in Period 1 or 2 will not proceed to higher CLTX-305 doses. Subjects with cCa values between 10-10.5 mg/dL during dose escalation in Period 1, will have their doses increased by 30 mg only. Subjects who achieve cCa>10-10.5 mg/dL in Periods 1 and >10 mg/dL in Period 2 will have their dose maintained or reduced and undergo final frequent sampling as appropriate.

### Dose Modifications

See above for general principles of dosing and titration limitations based on toxicity or hypercalcemia. See Example 7 for details of the dosing guidance algorithm for each Period.

Dose modifications should be made according to the protocol-specified guidance/algorithm described in Example 7 based on cCa thresholds of >10-10.5 mg/dL in Period 1 and 2. Similarly, titration in outpatient Period 3 will be based primarily on periodic assessment of cCa and where cCa>10 mg/dL would also require dose modification. In Period 3 dose modification may include reductions in CLTX-305 and/or changes to the oral calcium supplement regimen.

### Drug Administration

During Period 1 and Period 2 inpatient NIH CC stays, on days CLTX-305 is given once a day, subjects will receive one dose in the morning that may be administered within 30 minutes prior to breakfast with about 8 ounces of water. On days CLTX-305 is given twice a day, subjects will receive one dose in the morning and the second dose in the evening and can be administered within 30 minutes prior to breakfast and dinner with about 8 ounces of water.

During Period 3 subjects will be instructed by the investigator/designee to take one dose of CLTX-305 in the morning and the second dose in the evening. Taking the dose within 30 minutes of breakfast and dinner is suggested but not required.

### 6.2 Preparation/Handling/Storage/Accountability

### Acquisition and Accountability

The site research pharmacist or delegated personnel will maintain an accurate record of the receipt of the CLTX-305 shipped by the Sponsor, including the date and quantity received. In addition, an accurate drug disposition record will be kept that specifies the amount to be administered to each subject, the date of dispensation, and any amount returned. This inventory record must be available for inspection at any time, and copies of this record will be provided to the Sponsor at the conclusion of the study. At the completion of the study, the site research pharmacist or delegated personnel will provide the Sponsor with a complete record of CLTX-305 accountability.

### Formulation, Appearance, Packaging, and Labeling

The CLTX-305 will be provided as white film-coated tablets containing the active ingredient encaleret provided in 10, 30 and 60 mg doses.

The tablets contain the following excipients: mannitol, microcrystalline cellulose, colloidal silicon dioxide, croscarmellose sodium, sucrose fatty acid esters, hydroxypropyl methylcellulose, magnesium stearate, macrogol, and titanium oxide.

### Product Storage and Stability

The Sponsor will provide the study center with drug supplies. The CLTX-305 is to be stored at controlled room temperature between 20° C. and 25° C. with excursions permitted between 15° C. and 30° C. in the sponsor provided bottles.

In Periods 1 and 2, all subjects will receive CLTX-305 only as dispensed and administered by investigative site staff at the NIH CC. All unused CLTX-305 must be returned to the Sponsor or destroyed on-site with approval by the Sponsor, after a final CLTX-305 accountability has been completed. If unused CLTX-305 is not returned to the Sponsor, proof of destruction must be provided to the Sponsor.

In Period 3 subjects will be given CLTX-305 to allow outpatient BID self-administration at an individualized starting dose with overage to allow for dose up-titration at the discretion of the investigator, between NIH CC visits. If needed, additional CLTX-305 (encaleret) may be mailed to the subject by the NIH CC pharmacy per standard NIH CC pharmacy procedures. Subjects will return unused CLTX-305 at each designated NIH CC visit which will be reviewed for accountability and an estimate of compliance. Subjects must return unused CLTX-305 to the NIH CC at their next visit. CLTX-305 accountability will be performed by the NIH pharmacist or delegated personnel. After a final CLTX-305 accountability is completed any unused CLTX-305 must be returned to Sponsor or destroyed on-site with approval by the Sponsor and in accordance to procedure at NIH. If unused CLTX-305 is not returned to the Sponsor, proof of destruction must be provided to the Sponsor.

### 6.3 Measures to Minimize Bias: Randomization and Blinding

Not applicable. This is an open-label study.

### 6.4 Study Intervention Compliance

For inpatient Periods 1 and 2, CLTX-305 will be administered by site staff and recorded. The time of CLTX-305 administration will be documented in the appropriate CRF. Scheduled oral administration of study drug will occur at the study site under observation by study staff or designee, thus ensuring study drug compliance. During outpatient Period 3, subjects will self-administer CLTX-305 and should be giving instruction on dosing, timing of doses and timing of meal. In respect to CLTX-305 administration refer to section: “Drug Administration.” At each study visit and during telephone contacts, site staff will review and record compliance (e.g. document missed doses) and review and record any dose changes.

### 6.5 Concomitant Therapy

At Screening, the study staff at NIH will question each subject specifically on the use of all concomitant medications and record the medication, dosage, and duration of use in the appropriate CRF. In general, upon admission and throughout the study, site staff will monitor, record, and administer all concomitant medications during the inpatient stay.

All subjects will be instructed to stop taking calcitriol on Day −1, the day of admission, but continue their oral calcium supplement regimen. Subjects will receive at least 1,000 mg total daily calcium derived from diet and supplementation throughout the treatment period.

Subjects being treated with thiazide diuretics may be enrolled if they are willing and able to discontinue thiazides 5 half-lives prior to initiation of CLTX-305 and during the study treatment period. When the thiazide is being used an antihypertensive, alternative therapy will be offered.

Subjects being treated with strong CYP3A4 inhibitors (including clarithromycin, telithromycin, nefazodone, itraconazole, ketoconazole, atazanavir, darunavir, indinavir, lopinavir, nelfinavir, ritonavir, saquinavir, tipranavir), if clinically appropriate, should discontinue these medications during the screening period at least 5 half-lives prior to initiation of CLTX-305 and during the study treatment period. Otherwise, subjects being treated with strong CYP3A4 inhibitors should remain on stable doses throughout the trial.

### Example 7: A Phase 2b, Open-Label Dose-Ranging Study—Study Assessments

### 7.1 Treatment Period

### 7.1.1 Period 1 (Single Ascending Dose Escalation and BID PK/PD Profile)—Cohort 1

For subjects whose screening visit is >21 days before initiation of study drug, outpatient laboratory testing will be performed 10-14 days prior to admission for blood and urine chemistry to be reviewed by the NIH investigators.

All subjects will be instructed to stop taking calcitriol on Day −1, the day of admission, but continue their oral calcium supplement regimen until admitted.

Day −1 (Admission): Eligible subjects in Cohort 1 will be admitted to the NIH CC the day prior to dosing in Period 1.

Days 1, 2 and 3 (QD Dose Escalations): Up to 8 subjects (minimum of 5) will undergo a single-dose, dose-escalation over the first 3 days of admission. Morning doses of 30, 90 and 180 mg will be administered on Days 1, 2 and 3, respectively, accompanied by frequent blood and urine sampling for 24-hours for PK/PD measures (For details of PK/PD assays and timepoints as shown in Table 4A-Table 4C, Table 5, and Table 6A-Table 6C CLTX-305 will be administered in the morning, with water, within 30 minutes prior to breakfast. If dose escalation is limited due to tolerability or hypercalcemia, the investigator, in consultation with sponsor, may choose to repeat the last well-tolerated dose on Days 2 and 3, or down-titrate, as appropriate (See below for dosing guidance algorithm).

Days 4 and 5 (BID Dose Escalation): Based on each subject's individual PD responses to QD dose-escalation, an individualized dose of CLTX-305 will be chosen for BID dosing on Days 4 and 5. CLTX-305 will be administered once in the morning and once in the evening, within 30 minutes prior to breakfast and dinner. The dose selected should be based on each subject's response in cCa and/or PTH levels during the prior QD dosing (Days 1-3). The investigator, in consultation with the sponsor, will likely choose either 90 or 180 mg administered BID (for total daily doses of 180 and 360 mg, respectively) but will decide according to the following criteria for each subject;
The lowest dose that resulted in an increase in cCa by ≥1 mg/dL during the dosing interval, or the lowest dose that maintained cCa in the normal range without exceeding the upper limit of the reference range over the dosing interval

### AND/OR:

Peak iPTH 150-300 pg/mL and/or
Sustained elevations in iPTH levels≥50 pg/ml (ideally for up to 12 hours)

If no single QD dose meets the above criteria for a given subject, then the investigator should choose the highest QD dose that was well tolerated and begin administration of that dose given once in the morning and once in the evening recommended within 30 minutes prior to meals (BID) starting on the morning of Day 4.

Monitoring of cCa and iPTH will occur during BID dosing on Days 4 and 5 with frequent blood and urine sampling for PK/PD. For details on the timepoints and assays for PK/PD samples on Day 5 as specified in Table 6A-Table 6C. The total blood volume collected during Period 1 for each subject will be approximately 524 mL. If a subject has a low body weight (<50 kg for adults or <55 kg for adolescents), the blood volume taken will be reduced to remain within the NIH guidelines of 10.5 mL/kg/8 w (adult) and 9.5 mL/kg/8 w (pediatric). The last dose of study medication will occur in the evening of Day 5.

A schematic of dose modifications based on the guidance described above is shown in FIG. 5.

Day 6 (Discharge): After the PK/PD blood sampling is collected on the morning of Day 6 all subjects will resume their prior conventional treatment regimen. During Day 6, cCa levels will be monitored throughout the day. This period can be extended if additional monitoring or treatment (e.g., to stabilize calcium) is needed prior to discharge.

Outpatient testing done in Period 1 on day 7-8 (˜24-48 hours after expected discharge from the NIH clinical center) will ideally be collected at the NIH CC. If this is not practical, with the subject's permission, contact information for the subject will be provided to a sponsor-contracted healthcare service to schedule a mutually agreeable time for sample collection in the home. Blood samples collected will be evaluated for CLTX-305 concentrations as well as for blood calcium, magnesium, phosphate, 1,25-(OH)₂ Vitamin D and PTH. This will allow both an evaluation of calcium homeostasis as well as a final PK sample while CLTX-305 concentrations are washing-out after Period 1. Given that the half-life of CLTX-305 at steady state is ˜10-14 hours, the PK sample will represent a timepoint between 40-70 hours after the last dose of CLTX-305 (administered in the evening of Day 5). A telephone contact will be conducted on day 9-11 or once the calcium results are available to be reviewed with investigative site staff.

In order to characterize both the pre-study calcemic status as well as changes associated with experimental interventions, the protocol will rely on frequent measurement of albumin-corrected blood calcium concentrations. Assays will be conducted according to specifications of the NIH Clinical Center Laboratory, Bethesda, Md. The main indicator of normal versus abnormal blood calcium levels will be based on the correction for serum or plasma albumin according to the formula corrected calcium (cCa)=measured total calcium (mg/dL)+0.8(4−albumin (g/dL)) OR measured total calcium (mmol/L)+0.2(4−albumin (g/dL)), recognizing that the total calcium reported in SI units (mmol/L) can be converted to conventional units (mg/dL) by multiplying by 4. Given that enrolled subjects are expected to have both normal and stable albumin levels, the measures of total calcium and cCa are likely to be similar throughout the time course of this study. Ionized calcium assays will also be available at the NIH Clinical Center investigative site and can be measured either in the event of severe hypocalcemia or if there is any question about the accuracy of the cCa measure. Collection of ionized calcium will occur at the discretion of the expert investigators based on the clinical scenario on a case-by-case basis.

### 7.1.2 Period 2 (BID Dosing)—Cohorts 1 and 2

For cohort 1, 10-14 days before admission to the NIH CC, subjects will undergo outpatient laboratory testing for blood and urine chemistry. For cohort 2 subjects whose screening visit is >21 days before initiation of study drug, outpatient laboratory testing will be performed 10-14 days prior to admission for blood and urine chemistry. Results will be reviewed by the NIH investigators.

All subjects will be instructed to stop taking calcitriol on Day −1, the day of admission, but continue their oral calcium supplement regimen.

Day −1 (Admission): Eligible subjects will be admitted to the NIH Clinical Center the day prior to dosing.

Cohort 1—Subjects in Cohort 1 who complete Period 1 will be scheduled to return for Period 2 after at least 8 to 12 weeks (to recover blood volume and hematocrit levels). BID dosing will be individualized based on each subject's prior responses to CLTX-305 during Period 1.

The first 7 subjects who have completed CLTX-305-201 Period 2 and entered into Period 3 have had a wide range of CLTX-305 dose requirements from 10 mg once daily to 180 mg BID suggesting that CLTX-305 (encaleret) dosing is highly individualized. As a result, all remaining subjects in Period 2 will receive an initial CLTX-305 90 mg BID dose for at least 3-4 doses for evaluation of safety, tolerability and the ability to modulate cCa levels. The CLTX-305 dose will be up- or down-titrated as needed to maintain cCa levels below 10 mg/dL and blood phosphorus levels above the lower limit of normal. Dosing will be adjusted in 10, 30, or 60 mg increments based on the available CLTX-305 (encaleret) tablet strengths. A frequent sampling test day with additional serial blood and urine sampling to assess 24-hour PK/PD profiles will be performed on Day 5.

If DL1 is insufficient to raise or maintain cCa in the normal reference range within 48 hours, then the dose will be increased to dose level 2 (DL2) BID on Days 3, 4 and 5 including a frequent sampling test day (Day 5) with additional serial blood and urine sampling to assess 24-hour PK/PD profiles. For details on the PK/PD timepoints during frequent sampling on Day 5 as specified in Table 6A-Table 6C. The total blood volume collected during Period 2 for each subject will be approximately 458 mL. If a subject has a low body weight (<43 kg for adults or <48 kg for adolescents), the blood volume taken will be reduced to remain within the NIH guidelines of 10.5 mL/kg/8 w (adult) and 9.5 mL/kg/8 w (pediatric). If, at any time, the CLTX-305 dose is associated with hypercalcemia (cCa>10 mg/dL), then the dose should be reduced to a lower dose level.

An example of dose titration based on these simple rules is shown in FIG. 6:

Cohort 2—Cohort 2 will commence after Cohort 1 has completed Period 1 and the PD dose-responses have been reviewed. Subjects will receive an initial CLTX-305 90 mg BID dose for at least 3-4 doses for evaluation of safety, tolerability and the ability to modulate cCa levels. The CLTX-305 (encaleret) dose will be up- or down-titrated as needed to maintain cCa levels below 10 mg/dL and blood phosphorus levels above the lower limit of normal. Dosing will be adjusted in 10, 30, or 60 mg increments based on the available CLTX-305 (encaleret) tablet strengths.

Subjects will take the final dose of study medication in the evening of Day 5. After the final serial PK/PD blood sampling on the morning of Day 6, all subjects completing Period 2 will be eligible for participation in Period 3 to receive CLTX-305 at the same BID dose (or lower) in an outpatient setting. Subjects entering Period 3 will be discharged from NIH Clinical Center on the dose determined by the investigators to be effective and tolerated for each individual during Period 2.

If a subject does not participate in Period 3, the subject will resume their prior conventional outpatient treatment regimen on Day 6 prior to discharge.

In order to characterize both the pre-study calcemic status as well as changes associated with experimental interventions, the protocol will rely on frequent measurement of albumin-corrected blood calcium concentrations. Assays will be conducted according to specifications of the NIH Clinical Center Laboratory, Bethesda, Md. The main indicator of normal versus abnormal blood calcium levels will be based on the correction for serum or plasma albumin according to the formula corrected calcium (cCa)=measured total calcium (mg/dL)+0.8(4−albumin (g/dL)) OR measured total calcium (mmol/L)+0.2(4−albumin (g/dL)), recognizing that the total calcium reported in SI units (mmol/L) can be converted to conventional units (mg/dL) by multiplying by 4. Given that enrolled subjects are expected to have both normal and stable albumin levels, the measures of total calcium and cCa are likely to be similar throughout the time course of this study. Ionized calcium assays will also be available at the NIH Clinical Center investigative site and can be measured either in the event of severe hypocalcemia or if there is any question about the accuracy of the cCa measure. Collection of ionized calcium will occur at the discretion of the expert investigators based on the clinical scenario on a case-by-case basis.

### 7.1.3 Period 3 (Cohort 1 and Cohort 2)—Outpatient Dosing

Period 3 will include a titration phase of approximately 12 weeks and a maintenance phase of approximately 12 weeks for a total outpatient exposure to CLTX-305 of approximately 24 weeks. In Period 3, the initial outpatient dose of CLTX-305 will be based on the results from inpatient Periods 1 and 2. Outpatient titration will be conducted by the NIH investigators with the goal of optimizing the CLTX-305 dose without calcitriol, targeting normal cCa and phosphorus concentrations while minimizing the need for calcium supplements, avoiding symptoms of hypo- or hypercalcemia, and minimizing the extent of hypercalciuria. If blood cCa levels are >10.5 mg/dL, the NIH investigators can consider holding the CLTX-305 (encaleret) dose and restarting CLTX-305 (encaleret) at a lower dose after blood cCa has decreased to <10 mg/dL. Oral calcium supplementation may be used as needed on top of a minimum initial intake of at least 1000 mg daily.

Study visits include inpatient NIH visits every 8 weeks for PK/PD assessments, evaluation of safety, and recording of AEs and concomitant medications. Subjects will be contacted by telephone per protocol at intervals during titration and maintenance, to review the results of outpatient laboratory blood and urine assessments, confirm adequacy of the current regimen of study medication (including supplements), inquire about hypo/hypercalcemia symptoms, and assess for AEs. The total blood volume collected over 24 weeks during Period 3 for each subject will be approximately 540 mL.

### 7.1.4 Follow-Up and Early Termination

Subjects will receive a follow-up call 30±7 days after their last dose of the study medication for assessment of AEs.

If subjects discontinue from taking part in the study or are terminated early from the study, they should have all safety assessments for Early Termination (ET) as listed in the Schedules of Activities as described in Example 1: 1.2.

When a subject discontinues or withdraws from taking study medication, they will revert to their prior outpatient regimen of oral calcium and active Vitamin D to address their underlying ADH1 and maintain cCa blood concentrations as they return to their prior clinical care providers. Subjects will be instructed to undergo outpatient laboratory testing of their blood calcium within 1-3 days of discontinuing study medication and to have those results sent to NIH investigators for review. Study investigator should contact the subject to review the blood calcium results and advise on optimizing their outpatient clinical management.

### 7.2 Efficacy Assessments

### 7.2.1 Pharmacokinetic and Pharmacodynamic Blood Sampling

All PK blood samples will be analyzed by the CRO contracted by the Sponsor. All PD blood samples will be managed by the NIH laboratory. Details for sample processing and handling are provided in the Study Procedures Manual. The times for collection of these samples are detailed in the Schedule of Activities in Example 1: 1.2.

The key efficacy assessment is the cCa concentrations over time.

For a tabular listing of PK/PD and safety analytes in Blood and Urine, See 7.4.3.

### 7.2.2 Timed Interval and 24-Hour Urine

The times for collection of these samples are detailed in the Schedule of Activities in Table 1, Table 2, Table 3, Table 4A-Table 4C, and Table 6A-Table 6C of Example 1.

### 7.2.3 Samples for Genetic/Genomic Analysis

For subjects with no prior documented activating mutation in the CASR or an affected first-degree relative (with associated documentation collected and filed in subject's study binder), the diagnosis of ADH1 will be confirmed by genetic testing at screening.

If a subject's CASR mutation has not been confirmed at Screening, blood samples will be collected for somatic analysis of the CASR gene.

If no pathogenic variant in the CASR is identified, additional testing for other genetic forms of hypoparathyroidism may be offered to the subject.

No additional genetic testing is required as part of this protocol.

### 7.3 Safety and Other Assessments

### Physical Examinations

At the times detailed in the Schedule of Activities in Example 1, subjects will undergo a complete physical examination (PE), which is to be completed by a physician or an appropriately trained health professional. Any abnormal physical examination finding that is deemed clinically important (i.e., is associated with symptoms and/or requires medical intervention) will be recorded as an AE.

### Vital Signs

Vital signs will be assessed in accordance to times and details indicated in the Schedule of Activities in Table 1, Table 2, and Table 3 of Example 1. Any abnormal vital sign that is deemed clinically significant (i.e., is associated with symptoms and/or requires medical intervention) will be recorded as an AE.

### Clinical Laboratory Determinations

Blood and urine samples for clinical laboratory tests will be collected at the times detailed in the Schedule of Activities of Example 1. At Screening, the samples will be collected at the NIH Clinical Center. Additional testing during the Screening period may be conducted at a outpatient laboratory near the subject. All laboratory tests (PD and safety) during the Treatment Periods 1 and 2 will be measured at the NIH Clinical Center.

Outpatient testing done in Period 1 on day 7-8 (˜24-48 hours after expected discharge from the NIH clinical center) will ideally be collected at the NIH CC. If this is not practical, with the subject's permission, contact information for the subject will be provided to a sponsor-contracted healthcare service to schedule a mutually agreeable time for sample collection in the home. Blood samples collected will be evaluated for CLTX-305 concentrations as well as for blood calcium, magnesium, phosphate, 1,25-(OH)₂ Vitamin D and PTH. This will allow both an evaluation of calcium homeostasis as well as a final PK parameter while CLTX-305 concentrations are washing-out after Period 1. Given that the half-life of CLTX-305 at steady state is ˜10-14 hours, the PK sample will represent a timepoint between 40-70 hours after the last dose of CLTX-305 (administered in the evening of Day 5). A telephone contact will be conducted on day 9-11 or once the calcium results are available to be reviewed with investigative site staff.

Laboratory testing during outpatient Period 3 will include outpatient laboratory collection of blood for calcium and calcium-related analytes including safety laboratory tests.

The investigator will assess the clinical significance of values outside the reference ranges provided by the laboratory.

Samples will be collected, processed, and stored according to the instructions provided in the Laboratory Manual.

The investigator or qualified sub-investigator will review all laboratory results for clinical significance. Any laboratory result deemed clinically significant (i.e., is associated with signs and symptoms and/or requires medical intervention) will be recorded as an AE.

### Electrocardiograms

A standard 12-lead ECG will be assessed at the times detailed in the Schedule of Activities of Example 1. ECGs will be performed in the supine position after a 5-minute rest. The investigator or qualified sub-investigator will review all ECG interpretations and interval duration measurements for clinical significance. Any ECG interpretation deemed to be clinically significant (i.e., is associated with symptoms and/or requires medical intervention) will be reported as an AE.

### Dual-Energy X-Ray Absorptiometry (DXA)

Bone densitometry of the spine, hip, distal radius, and total body will be performed by DXA at Screening during Periods 1 and 2, at Week 24 during Period 3 per the Schedule of Activities of Example 1.

Radiation exposure. This research study involves exposure to radiation from one DXA scan performed at the screening visit and Week 24 during Period 3. This radiation exposure is not necessary for medical care and is for research purposes only. The amount of radiation participants will receive in this study is well below the dose guidelines established by the NIH Radiation Safety Committee for child (less than 500 mrem per year) or adult (less than 5000 mrem per year) research subjects. The effective dose that participants will receive from participation in this research study is less than two millirem. This protocol has been approved by the Radiation Safety Committee.

### 7.4 Adverse Events and Serious Adverse Events

All Adverse events are reported according to the study protocol.

### 7.4.1 Events of Special Interest

### Hypocalcemia in ADH1

The main goal of CLTX-305 treatment in subjects with ADH1 is to raise serum calcium levels. Hypocalcemia is not an expected side-effect of treatment with CLTX-305 but it is a complication of the underlying disease being treated. As such, hypocalcemia may develop whenever the underlying disease is inadequately treated and might occur due to either inadequate or missed doses of a specific, effective medication.

The current study involves reducing the dose of current maintenance medications or suspending their use entirely (i.e. oral calcium supplements and calcitriol), to facilitate dose-finding with the CLTX-305 and potentially establishing clinical proof-of-concept. Under these circumstances there is a risk that subjects could experience symptoms of hypocalcemia prior to achieving an effective dose of CLTX-305. In order to mitigate this risk, the study design emphasizes safety foremost. All changes to oral maintenance medications in conjunction with CLTX-305 dosing will be conducted with participants in residence at the Clinical Center in the first two periods, with multiple daily measurements of blood calcium and close supervision by health professionals with experience caring for individuals with ADH1. In addition to these precautions, monitoring for and capture of events suggestive of or consistent with hypocalcemia will be reviewed as an AE of special interest as described below.

### Assessment of Blood Calcium Concentrations

In order to characterize both the pre-study calcemic status as well as changes associated with experimental interventions, the protocol will rely on frequent measurement of albumin-corrected blood calcium concentrations. Assays will be conducted according to specifications of the NIH Clinical Center Laboratory, Bethesda, Md. The main indicator of normal versus abnormal blood calcium levels will be based on the correction for serum or plasma albumin according to the formula corrected calcium (cCa)=measured total calcium (mg/dL)+0.8(4−albumin (g/dL)) OR measured total calcium (mmol/L)+0.2(4−albumin (g/dL)), recognizing that the total calcium reported in SI units (mmol/L) can be converted to conventional units (mg/dL) by multiplying by 4. Given that enrolled subjects are expected to have both normal and stable albumin levels, the measures of total calcium and cCa are likely to be similar throughout the time course of this study. Ionized calcium assays will also be available at the NIH Clinical Center investigative site and can be measured either in the event of severe hypocalcemia or if there is any question about the accuracy of the cCa measure. Collection of ionized calcium will occur at the discretion of the expert investigators based on the clinical scenario on a case-by-case basis.

### Adverse Events of Hypocalcemia: Symptomatic and Asymptomatic

Adverse events consistent with hypocalcemia will be classified as either symptomatic or asymptomatic, and assessed for severity, recognizing that there is a spectrum of clinical manifestations. Symptomatic hypocalcemia will be based on identification of symptoms consistent with hypocalcemia such as numbness/tingling of hands, feet or lips, muscle cramps/spasms/twitching, other weakness or lightheadedness and/or new or worsening anxiety, including anger or depression and neurocognitive signs of confusion/hallucinations accompanied by laboratory evidence of a drop in cCa. Relevant symptoms will be documented as blood calcium levels are checked to confirm calcium status. A Case Report Form (CRF) for symptomatic hypocalcemia as an AE of special interest will capture the details of each hypocalcemic event including but not limited to symptoms, concomitant cCa concentrations, precipitating factors, treatment, resolution, and action taken regarding study medication.

Asymptomatic hypocalcemia will also be captured as an AE of special interest based on defining a threshold value for cCa<7 mg/dL. Since cCa levels are sampled frequently throughout the inpatient period, asymptomatic low cCa results will be identified during routine cCa monitoring.

### Clinical Evaluation and Treatment of Hypocalcemia

Hypocalcemia can range in symptom severity from asymptomatic to life-threatening. Appropriate treatment and action regarding study medication and/or other interventions (e.g., urgent calcium supplementation) will primarily be based on clinical evaluation of severity and acuity. Cases of hypocalcemia in this trial, if they occur, are expected to be manageable with supportive treatment.

Different patients may have different sensitivities to low calcium levels but one advantage of conducting this initial safety and proof-of-concept trial at the NIH is that the expert investigators have significant experience in managing these patients as well as specific historical knowledge about the clinical course and management of many members of the expected cohort to be studied. The investigators performing the current study have decades of accumulated and institutional experience caring for these patients including management of hypocalcemia that is a consequence of the underlying disorder. This includes experience managing outpatient regimens of oral calcium and calcitriol to ensure patient functioning, well-being and quality of life, based on outpatient laboratory monitoring and assessment of symptoms.

Appropriate evaluations may include serial cCa measures, an initial and follow-up EKG as appropriate based on clinical assessment (bradycardia e.g.) and/or if cCa is <7 mg/dL.

Graded interventions according to the expert judgment of the NIH investigators will include standard measures such as oral calcium and/or calcitriol supplementation (see below) for mild-moderate events. In the case on unresolving or worsening hypocalcemia with more acute clinical manifestations including worsening neurocognitive deficits, seizures, tetany, hypotension or cardiac dysfunction, for example, appropriate urgent intervention, also according with standards of care, will be undertaken by the site, including administration of intravenous calcium gluconate as describe below.

Addition of Oral Calcium Supplements and/or Calcitriol

Since all subjects will be receiving at least 1000 mg elemental calcium by mouth per day throughout the trial, additional supplemental oral calcium in the range of 1,000-2,000 mg additional supplement may be given as required with close follow-up of cCa. Transient requirements for calcium supplementation and/or 1-2 doses of calcitriol (at doses based on each subjects' prior known sensitivity and dosing of outpatient calcitriol, if known, e.g. likely in the range of 0.5-2 mcg) resulting in stabilized calcium levels, need not interfere with per protocol dose escalation, particularly if required on Days 1 and 2 before CLTX-305 effective doses may have been achieved. More severe or prolonged hypocalcemia unresponsive to oral supplementation may require early termination from the study period at the judgement of the investigator. If calcium levels are not stable and if evidence of clinically significant hypocalcemia precludes further dosing in the study period, CLTX-305 dosing will be stopped and subjects may be re-started on their prior outpatient regimen of oral calcium and calcitriol and discharged when stable, with close follow-up. Such subjects may be eligible to return for either a repeat of the study period or to the next per protocol study period, at the discretion of the investigator and sponsor.

### Intravenous Calcium Supplementation

In the event of severe hypocalcemia, the NIH Clinical Center has the facilities to conduct advanced supportive care including intravenous fluid replacement, supplemental oxygen, continuous ECG monitoring, and bedside ionized calcium monitoring if required. Intravenous calcium may be given as clinically indicated.

### Example 8: A Phase 2b, Open-Label Dose-Ranging Study—Statistical Consideration

All analysis details will be provided in a formal statistical analysis plan (SAP).

### Statistical Hypothesis

All data will be descriptively analyzed with no hypothesis testing.

### Sample Size Determination

The sample size of up to 8 subjects in Period 1 (Cohort 1) and up to 8 additional subjects in Period 2 (Cohort 2), is consistent with conventional first-in-human single and multiple ascending studies (SAD and MAD) that generally enroll between 8-10 subjects per dose arm with escalation being based on safety and tolerability.

The sample size for the current study is not based on statistical testing of a formal powered hypothesis. The proposed clinical trial represents a re-purposing of CLTX-305, leveraging extensive exposure and safety data from the prior osteoporosis program, to conduct a modified single and multiple-ascending dose study in a new target population of patients with ADH1. However, a recent publication of a different experimental calcilytic agent, NPSP795 (Rebuts et. al., 2019), reported preliminary evidence for a dose-response on iPTH in subjects with ADH1 with a sample size of 5 subjects. The authors speculated that despite testing doses known to raise iPTH robustly in non-ADH1 subjects, larger doses would have been required to stimulate PTH in ADH1 subjects sufficiently to also see elevations in cCa. The current protocol is designed to explore a broad potential dose range for safety and tolerability, however, the proposed sample size of approximately 16 subjects may be sufficient to confirm a definitive proof-of-concept on either iPTH and/or cCa if effective doses are achieved.

### Populations for Analyses

Safety Population: All subjects who received at least one dose of study drug

PK Population: All subjects who received at least one dose of study drug and who have sufficient PK samples drawn to enable the calculation of PK parameters for CLTX-305.

PD Population: All subjects with PD data.

### Statistical Analyses

All analysis details will be provided in a formal statistical analysis plan (SAP).

### Demographics and Other Baseline Characteristics

Demographics and other baseline characteristics will be summarized by treatment.

### Safety Analyses

Safety and tolerability parameters will be summarized using descriptive statistics, where appropriate. All safety data will be provided in data listings.

AEs will be coded using the MedDRA dictionary. The incidence of each treatment-emergent AE (TEAE) will be summarized by system organ class, preferred term and treatment assignment. Multiple AEs mapped to the same preferred term will be counted once per subject. Concomitant medications will be coded using the WHO Drug Dictionary with generic term and Anatomical Therapeutic Chemical (ATC) code and summarized by ATC code, WHO Drug generic name, and treatment. Reasons for early termination will be summarized by treatment group assignment. Safety laboratory findings, vital signs, and 12-lead ECGs will be summarized descriptively and listed by treatment assignment and visit. Values and changes from baseline at scheduled time points will be summarized. Laboratory data will be listed and values and changes from baseline at each visit will be summarized. An additional listing of treatment-emergent laboratory abnormalities will be provided.

An AE (classified by preferred term) that occurs during the treatment period will be considered a TEAE if it was not present before the first dose of CLTX-305 or if it was present before the first dose of CLTX-305 but increased in severity during the treatment period. If more than 1 AE is reported before the first dose of CLTX-305 and is coded to the same preferred term, the AE with the greatest severity will be used as the benchmark for comparison with the AEs that were also coded to that preferred term and that occurred during the period. An AE that occurs more than 30±7 days after the last dose of CLTX-305 will not be counted as a TEAE.

### Pharmacokinetic Analyses

PK parameters will be calculated for each subject and summarized by treatment. Only subjects with sufficient data to calculate each PK parameter will be included in the summary of each PK endpoint.

### Pharmacodynamic Analyses

PD parameters will be calculated for each subject and summarized by treatment. Only subjects with sufficient data to calculate each PD parameter will be included in the analysis.

### Interim Analysis

Due to the open-label study design and quantitative laboratory analytes used to derive PK and PD endpoints, both the Sponsor and NIH investigator will be able to conduct joint periodic data reviews on an ongoing basis throughout the study.

Review of safety, tolerability and effects on iPTH and cCa will be conducted on Period 1 single-dose and multiple-dose (QD and BID) responses prior to commencement of Period 2.

### Example 9: Results of Period 1/Cohort 1 of the Study

Six adults with ADH1 due to four distinct activating variants of the CASR were studied in an ongoing, three period, Phase 2b, open-label, dose-ranging study of the calcilytic encaleret (CLTX-305), according to the protocol of Examples 1-8. The study of Period 1, cohort 1 was outlined in FIG. 2. Calcium, magnesium, and calcitriol supplements were discontinued at the start of Period 1, and subjects received sequential, increasing daily doses of encaleret for 3 days (30 mg, 90 mg, 180 mg) followed by 120 mg or 180 mg twice daily on day 4 and 5, while undergoing frequent blood and urine sampling.

Subject Characteristics: The baseline characteristics of the subjects are summarized in Table 9.

TABLE 9
Baseline Characteristics of the Subjects

| Characteristics | N = 6 | | Normal Range |
|---|---|---|---|
| Age, mean (range) | 40 | (22-60) | |
| Male, n (%) | 3 | (50%) | |
| Nephrocalcinosis, n (%) | 4 | (67%) | |
| ECG QTcB (msec) | 452 ± 9 | | <440 |
| Corrected calcium (mg/dL)* | 7.6 ± 0.6 | | 8.4-10.2 |
| Intact PTH (pg/mL)* | 3.4 ± 4.5 | | 15-65 |
| Phosphorous (mg/dL)* | 4.5 ± 0.7 | | 2.5-4.5 |
| Magnesium (mg/dL)* | 1.6 ± 0.4 | | 1.6-2.6 |
| 24 h Urine Calcium (mg/24 h) | 436 ± 255 | | <250-300 |
| Supplement Doses | | | |
| Elemental Calcium | 2317 | (800-4000) | |
| (mg/day) [mean (range)] | | | |
| Calcitriol (μg/day) | 0.9 | (0.5-2.0) | |
| [mean (range)] | | | |

ECG QTcB = electrocardiogram Bazett-corrected Q-T interval;
*Measurements taken pre-dose Day 1 (mean ± SD); and
CASR variants (n): C131Y (2), P221L (2), A840V (1), E604K (1).

### Pharmacodynamics, Pharmacokinetics, Efficacy, and Safety and Tolerability

The six subjects received CLTX-305 with the following doses: Day−1: 30 mg (QD); Day−2: 90 mg (QD); Day−3: 180 mg (QD); Day−4: 180 mg (BID); and Day−5: 180 mg (BID), except that one subject received second dose of 120 mg on Day−5.

The mean baseline PTH was 3.4±4.5 pg/mL (mean±SD; nl 10-65); on encaleret, there was a rapid, dose-dependent increase in PTH to a mean level of 64.8±49.6 pg/mL over 24 hours by day 5. Albumin-corrected blood calcium (cCa) increased from a baseline of 7.6±0.6 mg/dL (nl 8.4-10.2) to a 24-hour mean on day 5 of 9.0±0.5 mg/dL. Phosphorus decreased from a baseline of 4.5±0.7 mg/dL (nl 2.3-4.7) to a 24-hour day 5 mean of 2.9±0.5 mg/dL. Magnesium increased from a baseline of 1.6±0.4 mg/dL (nl 1.6-2.6) to 24-hour day 5 mean of 2.0±0.5 mg/dL. Blood calcium, phosphorus, and magnesium were mostly maintained within the normal range in ADH1 subjects by days 4 and 5. Twenty-four hour urine calcium was elevated at the screening visit while subjects were on conventional therapy (436±255 mg/day, nl<250-300) and decreased with increasing doses of encaleret to 63±127 mg/day on day 5. Urinary calcium excretion became normal in 3 subjects and undetectable in 3 subjects while on encaleret. Encaleret was well-tolerated, with no serious adverse events reported, as shown in Table 10.

TABLE 10
Summary of Adverse Events (AEs), n (%)

| Subjects with Serious AEs | 0 | (0% |
|---|---|---|
| Subjects with AEs | 5 | (83%) |
| Mild | 5 | (83% |
| Moderate | 0 | (0%) |
| Severe | 0 | (0%) |
| Number of AEs | 9 | |
| Mild | 9 | (100%) |
| Moderate | 0 | (0%) |
| Severe | 0 | (0%) |

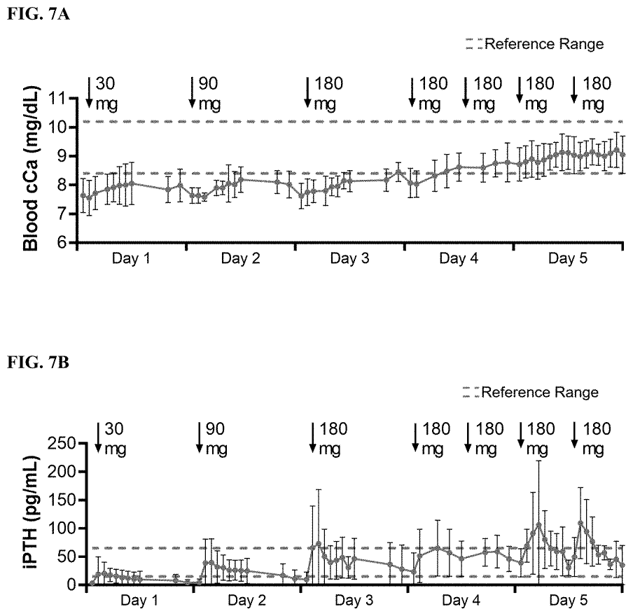

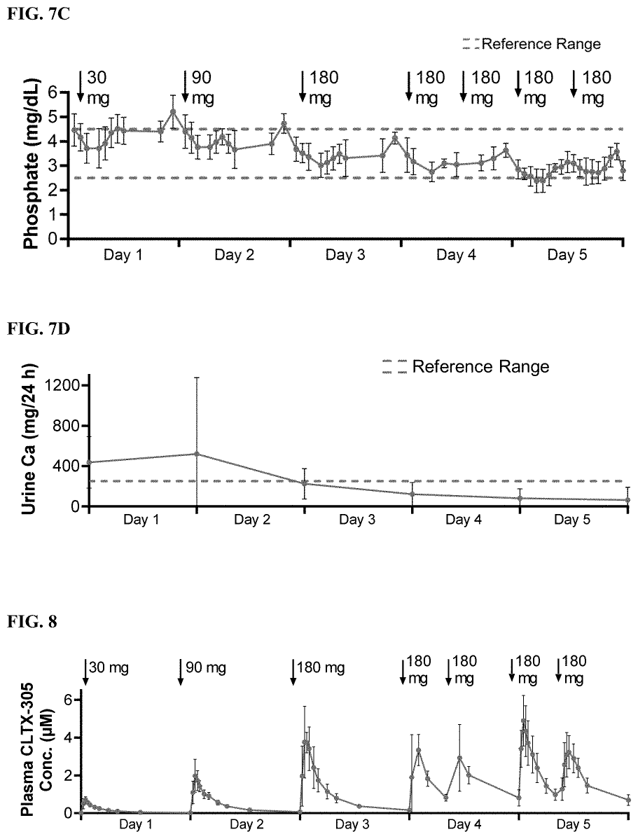

As shown in FIGS. 7A-7D, mineral homeostasis was normalized during Period 1 of the study [mean±SD]. FIG. 7A shows blood calcium levels (mg/dL); FIG. 7B shows intact PTH levels (pg/mL); FIG. 7C shows phosphorous levels (mg/dL); and FIG. 7D shows 24 h Urine calcium levels (mg/24 h).

As shown in FIG. 8, pharmacokinetic profile of CLTX-305 demonstrated dose proportional increase in plasma exposure over Period 1 of the study [mean±SD].

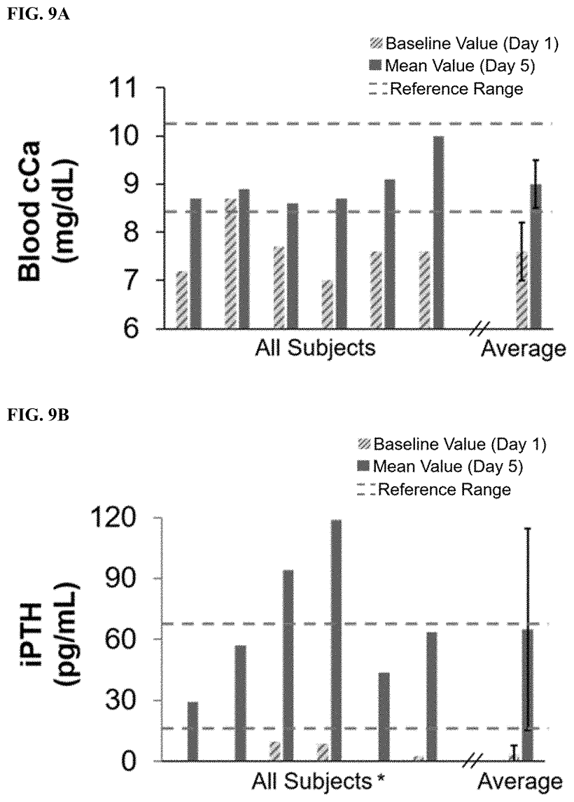

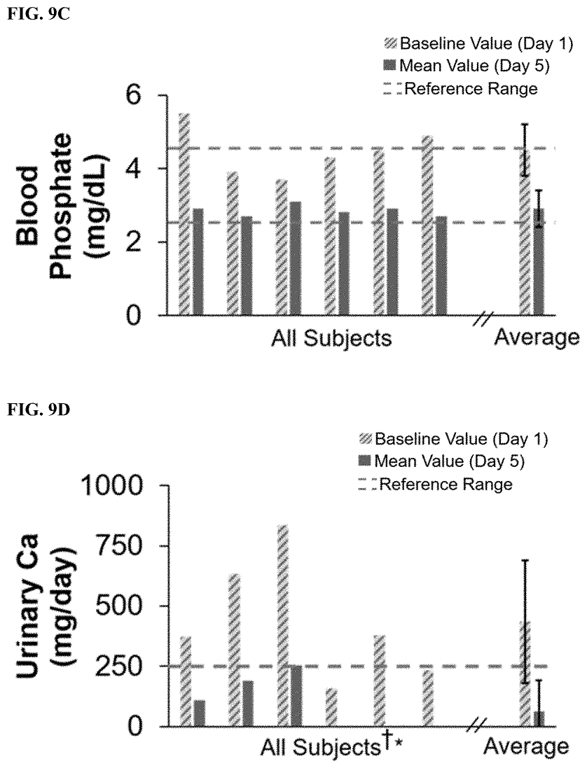

Blood mineral levels following 5-day dosing of CLTX-305 for individual subject and as an average are shown in FIGS. 9A-9D. FIG. 9A shows blood calcium levels (mg/dL); FIG. 9B shows intact PTH levels (pg/mL); FIG. 9C shows blood phosphorous levels (mg/dL); and

FIG. 9D shows 24 h Urine calcium levels (mg/day). († indicates that Day-4 values were shown while Day-5 values were unavailable. * indicates that values below limit of assay quantitation were plotted as “0”.)

Conclusion: Encaleret (CLTX-305) was well-tolerated when administered in escalating oral doses once or twice daily over 5 days, with no serious adverse events reported. Consistent changes from baseline in blood and urine mineral measurements provide preliminary proof-of-concept data that CLTX-305 may be an effective treatment for ADH1. Blood calcium, PTH, and phosphate were generally normalized and maintained within the normal range by day 5. Urinary calcium excretion became normal or undetectable in all subjects while on CLTX-305 and eucalcemic.

### Example 10: Results of Period 2/Cohort 2 of the Study

An additional seven subjects with ADH1 were studied as described in the preceding examples. Table 11 summarizes the baseline characteristics of these subjects.

TABLE 11
Baseline Characteristics of the Subjects

| Characteristics | N = 13 | | Normal Range |
|---|---|---|---|
| Age, mean (range) | 39 | (22-60) | |
| Female, n (%) | 8 | (62%) | |
| Nephrocalcinosis, n (%) | 10 | (77%) | |
| ECG QTcB (msec) | 452 ± 16 | | <440 |
| Albumin-corrected Calcium | 8.0 ± 0.7 | | 8.4-10.2 |
| (mg/dL)* | | | |
| Intact PTH (pg/mL)* | 2.8 ± 3.4 | | 15-65 |
| Phosphate (mg/dL)* | 5.1 ± 1.1 | | 2.3-4.7 |
| Magnesium (mg/dL)* | 1.8 ± 0.1 | | 1.6-2.6 |
| 24 h Urine Calcium (mg/24 h) | 425 ± 253 | | <250-300 |
| Supplement Doses | | | |
| Elemental Calcium | 2628 | (750-4800) | |
| (mg/day) [mean (range)] | | | |
| Calcitriol (μg/day) | 0.8 | (0.2-2.0) | |
| [mean (range)] | | | |

ECG QTcB = electrocardiogram Bazett-corrected Q-T interval;
*Measurements taken pre-dose Day 1 (mean ± SD) in Period 1 or Period 2; and
CASR variants (n): C131Y (2), P221L (2), E604K (1), A840V (3), F788C (1), T151M (1), Q245R (1), I692F (1), E228K (1).

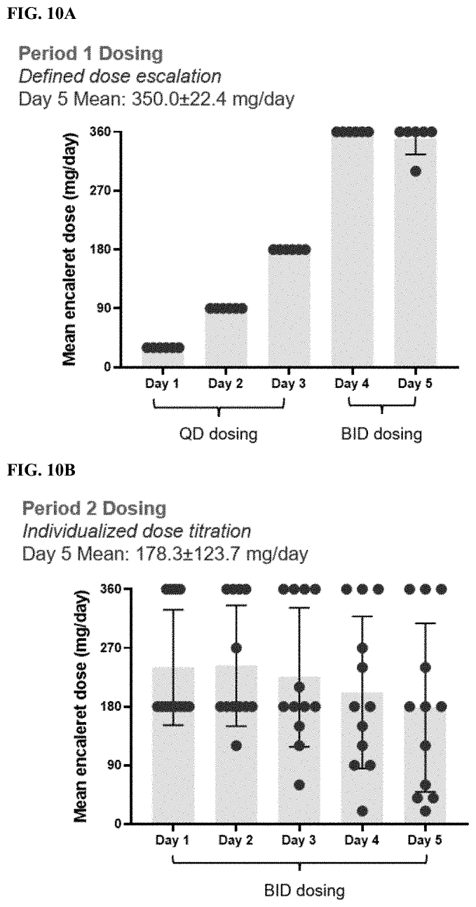

FIGS. 10A and 10B show the dosing summary for Periods 1 and 2 of the study. As shown in FIG. 10B, period 2 featured individualized dose titration.

As shown in Table 12, encaleret was well-tolerated in Period 2 with no serious adverse events reported.

TABLE 12
Summary of Adverse Events (AEs), n (%) in Period 2 (n = 13)

| Subjects with Serious AEs | 0 | (0%) |
|---|---|---|
| Subjects with AEs | 10 | (77%) |
| Mild | 10 | (77% |
| Moderate | 0 | (0%) |
| Severe | 0 | (0%) |
| Number of AEs | 12 | |
| Mild | 12 | (100%) |
| Moderate | 0 | (0%) |
| Severe | 0 | (0%) |

Blood mineral levels following 5-day dosing of CLTX-305 in Period 2 are consistent with those shown in FIGS. 9A-9D.

Conclusion: In 13 participants, encaleret normalized mean corrected blood calcium and 24-hour urine calcium excretion during Periods 1 and 2. Mean PTH increased and phosphate decreased into the normal range during Period 1 and 2. Compared with Period 1, individualized BID dosing in Period 2 resulted in a decrease in the mean Day 5 dose. Encaleret was well-tolerated when administered once or twice daily over 5 days, with no serious adverse events reported. Consistent improvements in mineral homeostasis suggest encaleret may be an effective treatment for ADH1. Outpatient evaluation of encaleret in this Phase 2b study remains ongoing.

Although the foregoing disclosure has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference. Where a conflict exists between the instant application and a reference provided herein, the instant application shall dominate.

What is claimed is:

1. A method of treating an autosomal dominant hypocalcemia type 1 (ADH1) comprising administering to a subject in need thereof a therapeutically effective amount of a compound represented by formula (I): 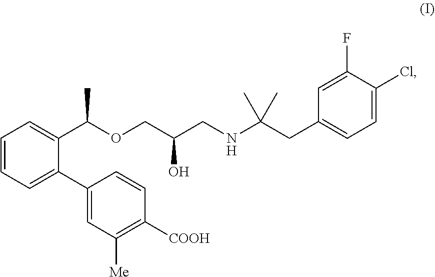 [SMILES: Cc1cc(-c2ccccc2[C@@H](C)OC[C@H](O)CNC(C)(C)Cc2ccc(Cl)c(F)c2)ccc1C(=O)O]
or a solvate, a hydrate, a pharmaceutically acceptable salt, or a combination thereof, wherein the therapeutically effective amount of the compound or the solvate, hydrate, pharmaceutically acceptable salt, or combination thereof increases a blood calcium concentration (cCa) to a range of about 7.5 milligrams per deciliter (mg/dL) to about 10.5 mg/dL.

2. The method of claim 1, wherein the compound is in a hemihydrate hemisulfate salt form as CLTX-305 represented by formula (Ia):

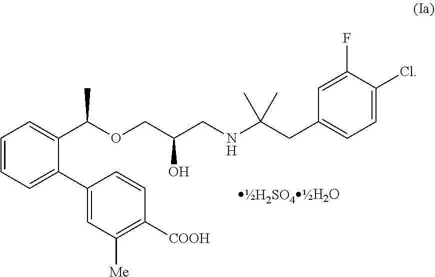 [SMILES: Cc1cc(-c2ccccc2[C@@H](C)OC[C@H](O)CNC(C)(C)Cc2ccc(Cl)c(F)c2)ccc1C(=O)O]

3. The method of claim 1 or 2, wherein the subject has hypocalcemia, hypoparathyroidism, hypercalciuria, hyperphosphatemia, and/or hypomagnesemia.

4. The method of any one of claims 1 to 3, wherein the subject has an activating mutation of the calcium-sensing receptor (CASR) gene.

5. The method of any one of claims 1 to 4, wherein the subject has 25-hydroxy-vitamin D in blood at a level of at least about 25 nanograms per milliliter (ng/mL).

6. The method of any one of claims 1 to 5, wherein the subject is not treated with calcitriol.

7. The method of any one of claims 1 to 6, wherein the subject receives a daily calcium intake of at least about 1000 mg from diet and/or supplementation.

8. The method of any one of claims 1 to 7, wherein the therapeutically effective amount is a total daily dosage of from about 10 mg to about 1800 mg, from about 10 mg to about 1200 mg, from about 10 mg to about 900 mg, from about 10 mg to about 600 mg, or from about 10 mg to about 360 mg of CLTX-305.

9. The method of claim 8, wherein the therapeutically effective amount is a total daily dosage of about 10 mg, 20 mg, 30 mg, 40 mg, 60 mg, 80 mg, 120 mg, 140 mg, 180 mg, 300 mg, 360 mg, 480 mg, or 720 mg of CLTX-305.

10. The method of any one of claims 1 to 9, wherein the compound of formula (I) or CLTX-305 is administered orally.

11. The method of any one of claims 1 to 10, wherein the compound of formula (I) or CLTX-305 is administered once, twice, three times, or four times daily.

12. The method of claim 11, wherein the compound of formula (I) or CLTX-305 is administered twice daily.

13. The method of any one of claims 1 to 12, wherein the therapeutically effective amount of the compound of formula (I) or CLTX-305 increases the blood calcium concentration (cCa) by at least about 1 mg/dL over a dosing interval.

14. The method of any one of claims 1 to 13, wherein the therapeutically effective amount of the compound of formula (I) or CLTX-305 increases intact parathyroid hormone (iPTH) in blood to a peak level of from about 150 picograms per milliliter (pg/mL) to about 300 pg/mL.

15. The method of any one of claims 1 to 14, wherein the therapeutically effective amount of the compound of formula (I) or CLTX-305 increases intact parathyroid hormone (iPTH) in blood to an elevated level of at least about 50 pg/mL.

16. The method of claim 15, wherein the iPTH is maintained at the elevated level of at least about 50 pg/mL for a period of 1-12 hours.

17. The method of claim 16, wherein the iPTH is maintained at the elevated level of at least about 50 pg/mL for a period of about 12 hours.

18. The method of any one of claims 1 to 17, wherein the subject is further evaluated for a level of magnesium, (Mg), phosphate (P), sodium (Na), potassium (K), creatinine (Cr), cAMP, and/or citrate in urine and/or a pH value of the urine.

19. The method of any one of claims 1 to 18, wherein the therapeutically effective amount of the compound of formula (I) or CLTX-305 maintains the blood calcium concentration (cCa) in a range of about 7.5 mg/dL to about 10.5 mg/dL for a period of at least 12 weeks without substantially adjusting the total daily dosage.

20. The method of any one of claims 1 to 19, further comprising administering an oral calcium supplementation in addition to the daily calcium intake.

21. A method of treating an autosomal dominant hypocalcemia type 1 (ADH1) comprising administering to a subject in need thereof a compound represented by formula (I): 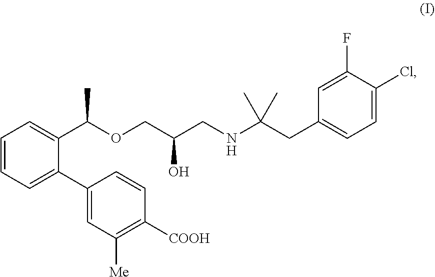 [SMILES: Cc1cc(-c2ccccc2[C@@H](C)OC[C@H](O)CNC(C)(C)Cc2ccc(Cl)c(F)c2)ccc1C(=O)O]
or a solvate, a hydrate, a pharmaceutically acceptable salt, or a combination thereof, according to one or more dosing regimens comprising a first dosing regimen, a second dosing regimen, and/or a third dosing regimen,
wherein:
1) the first dosing regimen comprises administering a first therapeutically effective amount of the compound or the solvate, hydrate, pharmaceutically acceptable salt, or combination thereof, wherein the first therapeutically effective amount increases a blood calcium concentration (cCa) up to a maximum cCa of about 10.5 milligrams per deciliter (mg/dL);
2) the second dosing regimen comprises administering a second therapeutically effective amount of the compound or the solvate, hydrate, pharmaceutically acceptable salt, or combination thereof, wherein the second therapeutically effective amount titrates the blood calcium concentration (cCa) to a range of about 7.5 mg/dL to about 10.5 mg/dL; and
3) the third dosing regimen comprises administering a third therapeutically effective amount of the compound or the solvate, hydrate, pharmaceutically acceptable salt, or combination thereof, wherein the third therapeutically effective amount maintains the blood calcium concentration (cCa) in a range of about 7.5 mg/dL to about 10.5 mg/dL for a period of at least 12 weeks.

22. The method of claim 21, wherein the compound of formula (I) is in a hemihydrate hemisulfate salt form as CLTX-305 represented by the formula:

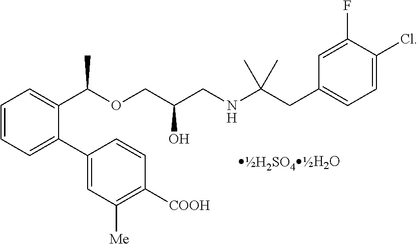 [SMILES: Cc1cc(-c2ccccc2[C@@H](C)OC[C@H](O)CNC(C)(C)Cc2ccc(Cl)c(F)c2)ccc1C(=O)O]

23. The method of claim 21 or 22, wherein the compound of formula (I) or CLTX-305 is administered orally.

24. The method of any one of claims 21 to 23, wherein the subject has hypocalcemia, hypoparathyroidism, hypercalciuria, hyperphosphatemia, and/or hypomagnesemia.

25. The method of any one of claims 21 to 24, wherein the subject has an activating mutation of the calcium-sensing receptor (CASR) gene.

26. The method of any one of claims 21 to 25, wherein the patient has 25-hydroxy-vitamin D in blood at a level of at least about 25 nanograms per milliliter (ng/mL).

27. The method of any one of claims 21 to 26, wherein the patient is not treated with calcitriol.

28. The method of any one of claims 21 to 27, wherein the patient receives a daily calcium intake of at least about 1000 mg either from diet or supplementation.

29. The method of any one of claims 21 to 28, wherein the first dosing regimen comprises a total daily dosage of at least about 10 mg, 20 mg, 30 mg, 40 mg, 60 mg, 80 mg, 90 mg, 120 mg, 140 mg, 180 mg, 300 mg, or 360 mg of CLTX-305.

30. The method of claim 29, wherein the first dosing regimen comprises an initial total daily dosage of about 30 mg of CLTX-305.

31. The method of any one of claims 21 to 30, wherein the first dosing regimen comprises administering CLTX-305 in a daily dosing frequency of 1 to 4 times daily.

32. The method of claim 31, wherein the first dosing regimen comprises administering CLTX-305 once daily for initial three days and twice daily for two days.

33. The method of any one of claims 21 to 32, wherein the first dosing regimen comprises:
a1) increasing the total daily dosage, when the blood calcium concentration (cCa) is less than a maximum cCa of about 10.5 mg/dL;
b1) decreasing the total daily dosage, when the blood calcium concentration (cCa) reaches the maximum cCa; and/or
c1) increasing the daily dosing frequency while maintaining the total daily dosage, when the blood calcium concentration (cCa) reaches the maximum cCa.

34. The method of claim 33, wherein the blood calcium concentration (cCa) is determined after each of one or more once-daily dosages and one or more twice-daily dosages.

35. The method of claim 34, wherein the total daily dosage is increased from about 30 mg to about 60 mg, from about 30 mg to about 90 mg, from about 10 mg to about 20 mg, from about 20 mg to about 40 mg, from about 60 mg to about 90 mg, from about 60 mg to about 120 mg, from about 90 mg to about 120 mg, from about 90 mg to about 180 mg, or from about 180 mg to about 360 mg of CLTX-305.

36. The method of claim 34, wherein the total daily dosage is decreased from about 30 mg to about 10 mg, from about 40 mg to about 20 mg, from about 60 mg to about 30 mg, from about 90 mg to about 60 mg, from about 90 mg to about 30 mg, from about 120 mg to about 90 mg, from about 120 mg to about 60 mg, from about 180 mg to about 120 mg, from about 180 mg to about 90 mg, from about 180 mg to about 60 mg, or from about 360 mg to about 180 mg of CLTX-305.

37. The method of claim 34, wherein the daily dosing frequency is increased from once to twice daily while maintaining the total daily dosage of about 30 mg, 60 mg, 90 mg, or 180 mg of CLTX-305.

38. The method of any one of claims 21 to 37, wherein the first dosing regimen further comprises:
i) selecting a lowest dosage among one or more once-daily dosages and administering the lowest dosage twice daily, provided that the one or more once-daily dosages meet a criteria selected from the group consisting of:
i-1) the blood calcium concentration (cCa) is increased by at least about 1 mg/dL over a dosing interval;
i-2) the blood calcium concentration (cCa) is maintained in a range of about 7.5 to about 10.5 mg/dL;
i-3) intact parathyroid hormone (iPTH) in blood is increased to a peak level of from about 150 pg/mL to about 300 pg/mL; and
i-4) intact parathyroid hormone (iPTH) in blood is increased to an elevated level of at least about 50 pg/mL and maintained for a period of about 12 hours,
or
ii) selecting a highest dosage among one or more once-daily dosages and administering the highest dosage twice daily, provided that the one or more once-daily dosages do not meet any one of criteria i-1) to i-4); and the highest dosage is tolerable in the subject.

39. The method of any one of claims 21 to 38, wherein the second dosing regimen comprises a total daily dosage of at least about 10 mg, 20 mg, 40 mg, 60 mg, 80 mg, 120 mg, 140 mg, 180 mg, 300 mg, 360 mg, 480 mg, or 720 mg of CLTX-305.

40. The method of claim 39, wherein the second dosing regimen comprises an initial total daily dosage of about 10 mg, 20 mg, 30 mg, 60 mg, 80 mg, 90 mg, 120 mg, 140 mg, 180 mg, 300 mg, 360 mg, 480 mg, or 720 mg of CLTX-305.

41. The method of any one of claims 21 to 40, wherein the second dosing regimen comprises administering CLTX-305 in a daily dosing frequency of 2 to 4 times daily.

42. The method of claim 41, wherein the second dosing regimen comprises administering CLTX-305 twice daily for five days.

43. The method of any one of claims 21 to 42, wherein the second dosing regimen comprises:
a2) maintaining the total daily dosage, when the blood calcium concentration (cCa) is maintained in a range of from about 7.5 mg/dL to about 10.5 mg/dL;
b2) increasing the total daily dosage, when the blood calcium concentration (cCa) is less than about 7.5 mg/dL; and/or
c2) decreasing the total daily dosage when the blood calcium concentration (cCa) is more than about 10.5 mg/dL.

44. The method of claim 43, wherein the blood calcium concentration (cCa) is determined after the first dosing regimen or after initial two days of the second dosing regimen.

45. The method of claim 44, wherein the total daily dosage is increased from about 20 mg to about 40 mg, from about 40 mg to about 60 mg, from about 60 mg to about 120 mg, from about 120 mg to about 180 mg, from about 180 mg to about 360 mg, from about 360 mg to about 480 mg, or from about 480 mg to about 720 mg of CLTX-305.

46. The method of claim 44, wherein the total daily dosage is decreased from about 40 mg to about 20 mg, from about 60 mg to about 40 mg, from about 120 mg to about 60 mg, from about 180 mg to about 120 mg, from about 360 mg to about 180 mg, or from about 480 mg to about 360 mg of CLTX-305.

47. The method of any one of claims 21 to 46, wherein the third dosing regimen comprises a total daily dosage of at least about 10 mg, 20 mg, 40 mg, 60 mg, 80 mg, 120 mg, 140 mg, 180 mg, 300 mg, 360 mg, 480 mg, or 720 mg of CLTX-305.

48. The method of claim 47, wherein the third dosing regimen comprises an initial total daily dosage of about 10 mg, 20 mg, 40 mg, 60 mg, 80 mg, 120 mg, 140 mg, 180 mg, 300 mg, 360 mg, 480 mg, or 720 mg of CLTX-305.

49. The method of any one of claims 21 to 48, wherein the third dosing regimen comprises administering CLTX-305 in a daily dosing frequency of 2 to 4 times daily.

50. The method of claim 49, wherein the third dosing regimen comprises administering CLTX-305 twice daily for at least 24 weeks.

51. The method of any one of claims 21 to 50, wherein the third dosing regimen comprises titrating the total daily dosage while maintaining the blood calcium concentration (cCa) in a range of from about 7.5 mg/dL to about 10.5 mg/dL.

52. The method of claim 51, wherein the third dosing regimen comprises:
a3) maintaining the total daily dosage when the blood calcium concentration (cCa) is in a range of from about 7.5 mg/dL to about 10.5 mg/dL;
b3) increasing the total daily dosage when the blood calcium concentration (cCa) is less than about 7.5 mg/dL; and/or
c3) decreasing the total daily dosage when the blood calcium concentration (cCa) is more than about 10.5 mg/dL.

53. The method of any one of claims 21, 47 to 52, wherein the third dosing regimen reduces symptoms associated with hypocalcemia and hypercalcemia, and minimizes hypercalciuria in the subject.

54. The method of any one of claims 21, 47 to 53, wherein the third dosing regimen maintains the blood calcium concentration (cCa) in a range of about 7.5 mg/dL to about 10.5 mg/dL for a period of at least 24 weeks.

55. The method of any one of claims 21, 47 to 53, wherein the third dosing regimen further comprises administering an oral calcium supplementation in addition to the daily calcium intake.

56. The method of any one of claims 21 to 55, wherein the subject is evaluated by one or more tests comprising blood analyses, urine analyses, and/or hematology tests.
